(19) United States
(12) Patent Application Publication
(10) Pub. No.: US 2007/0128589 A1
(43) Pub. Date: Jun. 7, 2007

(54) Substrates, sensors, and methods for assessing conditions in females

(76) Inventors: Mitchell C. Sanders, West Boylston, MA (US); Diane L. Ellis-Busby, Lancaster, MA (US); Jennifer M. Havard, Framingham, MA (US); John McCarty, Lyndonville, VT (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/487,141

(22) Filed: Jul. 13, 2006

Related U.S. Application Data
(60) Provisional application No. 60/782,167, filed on Mar. 13, 2006.
(60) Provisional application No. 60/732,036, filed on Oct. 31, 2005.
(60) Provisional application No. 60/699,133, filed on Jul. 13, 2005.

(51) Int. Cl. C12Q 1/70; C12Q 1/66; C12Q 1/37

(52) U.S. Cl. 435/5; 435/8; 435/23

(57) ABSTRACT

Described herein are substrates, methods, articles, and kits that are useful for detecting a condition in a female mammal. The substrates interact with one or more proteins (e.g., an enzyme) produced by a microorganism or the female animal. The substrates are labelled in order to produce a visible signal (e.g., a fluorescent glow and/or a visible change in color or hue) when modified by a protein produced by a microorganism of interest. The visible signal is used to assess a condition in the female mammal.

### RELATED APPLICATIONS

This application claims the benefit of U.S. Provisional Application No. 60/782,167, filed on Mar. 13, 2006, U.S. Provisional Application No. 60/732,036, filed on Oct. 31, 2005 and U.S. Provisional Application No. 60/699,133, filed on Jul. 13, 2005. The entire teachings of the above applications are incorporated herein by reference.

### BACKGROUND OF THE INVENTION

Bacterial vaginosis (BV) is a common disorder in women that results in the abnormal discharge of vaginal fluids. See Valore, E. V. et al., “Antimicrobial Components of Vaginal Fluid,” Am J Obstet Gynecol, 187:561-568 (2002) and Joesoef, M. R. et al., “Bacterial Vaginosis”, Clin Evid., (11):2054-63 (2004). Although this disease can be asymptomatic, BV can lead to more serious problems, including endometritis, pre-term births and low infant birth weight, urinary tract infections, pelvic inflammatory disease, post-gynecologic-surgery infections, cervicitis, and cervical intraepithelial neoplasia. See Alanen, A., “Does Screening Reduce Preterm Births?,” Br Med J, 329(7462):374 (2004), Honest, H. et al., “The Accuracy of Various Tests for Bacterial Vaginosis in Prediction Preterm Birth: a Systemic Review,” Int J Gynaecol Obstet, 111:409 (2004), Kiss, H. et al., “Prospective Randomised Controlled Trial of an Infection Screening Programme to Reduce the Rate of Preterm Delivery,” Br Med J, 329:371-375 (2004), Reid, G. et al., “Nucleic Acid-Based Diagnosis of Bacterial Vaginosis and Improved Management Using Probiotic Lactobacilli,” J Med Food., 7(2):223-8 (2004), and Rodriguez, R. et al., “Genital Infection and Infertility,” Enferm Infecc Microbiol Clin., 19:261-266 (2001). There is also some evidence to suggest that BV may also lead to an increased risk for viral sexually transmitted diseases, such as genital herpes (HSV-2) and human immunodeficiency virus (HIV). See Landers, D. V. et al., “Predictive Value of the Clinical Diagnosis of Lower Genital Tract Infection in Women,” Am J Obstet Gynecol, 190:1004-10 (2004), Pal, Z. et al., “Bacterial Vaginosis and Other Vaginal Infections,” Int J Gynaecol Obstet, 89:278-279 (2005), and Myer, L. et al., “Bacterial Vaginosis and Susceptibility to HIV Infection in South African Women: a Nested Case-Control Study,” J Infect Dis, 192:1372-1380 (2005).

Candidiasis (CV) is an opportunistic infection by Candida albicans that can turn pathogenic and cause infections. See DeLeon, E. M. et al., “Prevalence and Risk Factors for Vaginal Candida Colonization in Women With Type land Type 2 Diabetes,” BMC Infect Dis, 2 (2002), Nyirjesy, P. “Chronic Vulvovaginal Candidiasis,” Am Fam Physician, 63:697-702 (2001), Saavedra, M. et al., “Local Production of Chemokines During Experimental Vaginal Candidiasis,” Infect Immun, 5820-5826 (1999).

Trichomoniasis (TRIC), a protist, is a common form of vaginitis with approximately 3 million women infected each year. See Schwebke, J. R. et al., “Trichomoniasis,” Clin Microbiol Rev, 17(4):194-803 (2004).

One of the greatest health risks associated with vaginitis (BV, TRIC, and CV) is the increased risk of sexually transmitted diseases (STDs). See Msuya, S. E. et al., “Reproductive Tract Infections and the Risk of HIV Among Women in Moshy, Tanzania,” Acta Obstet Gynecol Scand, 81:886-893 (2002), Wiesenfeld, H. C. et al., “The Infrequent Use of Office-Based Diagnostic Tests for Vaginitis,” Am J Obstet Gynecol, 181:39-41 (1999), and Cosentino, L. A. et al., “Detection of Chlamydia trachomatis and Neisseria gonorrhoeae by Strand Displacement Amplification and Relevance of the Amplification Control for use With Vaginal Swab Specimens,” J Clin Microbiol, 3592-3596 (2003). Women are two times more likely to get HIV if they have BV because of the pH increase and loss of Lactobacilli, leading to a more favorable milieu for the virus to activate CD4 lymphocytes. See Mastromarino, P. et al., “Characterization and Selection of Vaginal Lactobacillus Strains for the Preparation of Vaginal Tablets,” J Appl Microbiol., 93(5):884-93 (2002). Vaginal infections of the reproductive tract have also been implicated in increasing the transmission of HIV by enhancing the shedding of the virus. See Sha, B. E. et al., “Female Genital-Tract HIV Load Correlates Inversely With Lactobacillus Species but Positively With Bacterial Vaginosis and Mycoplasma Hominis,” J Infect Dis., 191(1):25-32 (2005). Studies are being conducted to understand this relationship and provide an approach to decreasing HIV transmission. Vaginal infections can also lead to endometriosis, pelvic inflammatory disease, post surgical infections, pre term births, and low birth weights. See Stevens, A. O. et al., “Fetal Fibronectin and Bacterial Vaginosis are Associated With Preterm Birth in Women Who are Symptomatic for Preterm Labor,” Am J Obstet Gynecol, 190:1582-1589 (2004), Wilks, M. et al., “Identification and H₂O₂ Production of Vaginal Lactobacilli From Pregnant Women at High Risk of Preterm Birth and Relation With Outcome,” J Clin Microbiol., 42(2):713-7 (2004), and Lamont, R. F. et al., “Review of the Accuracy of various Diagnostic Tests for Bacterial Vaginosis to Predict Preterm Birth,” BJOG., 112(2):259-60 (2005).

Herpes Simplex Virus (HSV-2) is one of the leading causes of genital herpes. Genital herpes is a significant healthcare problem, with one in five adolescents and adults having the disease. The CDC estimates that 45 million people in the U.S. each year have HSV genital infections. HSV infections can be difficult to diagnose between outbreaks.

A need exists for methods and materials that can be used to assess and/or diagnose vaginitis (BV, CV and TRIC), and other conditions and states in females.

### SUMMARY OF THE INVENTION

It has been found that peptide substrates can be labelled in order to produce a visible signal (e.g., a fluorescent or luminescent glow and/or a visible change in color or hue) when modified by a protein. It has also been found that molecules (e.g., proteins secreted by microorganisms, expressed on the cell surface of microorganisms, or expressed on the surface of a cell infected with a microorganism or virus) can serve as markers for the detection of the presence or absence of a microorganism in a sample (e.g., a portion of tissue or urine or vaginal fluid) taken from a mammal (e.g., a human). Accordingly, the present invention features substrates that are modified by proteins, methods of detecting such a modification, methods for detecting proteins, and articles and kits incorporating substrates.

In some embodiments, this invention includes a method of assessing a condition in a female mammal (e.g., a human female). The methods comprise the steps of exposing an unmodified substrate to a sample under conditions that will result in a modification of the substrate and detecting the modification of the substrate or an absence of the modification of the substrate. The unmodified substrate includes, for example, a peptide and a calorimetric component that is coupled to the peptide, and the sample includes, for example, mammalian vaginal fluid or urine. The modification comprises cleaving the colorimetric component from the substrate and results in a visible signal. The modification or absence of the modification indicates a condition, such as a medical condition, in a female.

In other embodiments, this invention includes a peptide comprising at least one of the amino acid sequences selected from the group consisting of the sequence PFINETYAKFC (SEQ ID NO: 1), the sequence ITTTSSKHEHC (SEQ ID NO: 2), the sequence KPKAFXXX (SEQ ID NO: 3), the sequence VPGDPEAAEARRGQC (SEQ ID NO: 4), the sequence KPKAFLKGRR (SEQ ID NO: 5), the sequence KPKAFLKVGN (SEQ ID NO: 6), the sequence LYPILKKNQK (SEQ ID NO: 7), the sequence KPSIKPTPPY (SEQ ID NO: 8), the sequence QKTTIKKLKH (SEQ ID NO: 9), the sequence TPIQIHTILH (SEQ ID NO: 10), the sequence INLSKKQIYP (SEQ ID NO: 11), the sequence LYPSQNPVIK (SEQ ID NO: 12), and the sequence NITKKSTKII (SEQ ID NO: 13), the sequence NNPLPKIQKN (SEQ ID NO: 14), the sequence KNPKLQDHYI (SEQ ID NO: 15), the sequence QINKALKQPK (SEQ ID NO: 16), the sequence QIPKSLHPIT (SEQ ID NO: 17), the sequence LHNYVLLRNIL (SEQ ID NO: 18), the sequence SKQQDIIKKY (SEQ ID NO: 19), the sequence NKTNKTKHAY (SEQ ID NO: 20), the sequence QRTTIRRLRH (SEQ ID NO: 21), the sequence ASNAEAGALVNASSAAHVDV (SEQ ID NO: 22) and/or a modified peptide, for example, one containing one or more conserved amino acid substitutions. In some embodiments, the peptide is a variant or a fragment of a peptide described herein.

In further embodiments, this invention includes sensors for detecting the presence or absence of a protein. The sensors comprise a peptide that specifically reacts with a protein (e.g., a protein produced by a microorganism) and a colorimetric component or enzyme coupled to the peptide.

In still more embodiments, this invention includes a kit for assessing a medical condition in a female mammal. The kits comprise a sensor of the invention and at least one reagent. In one embodiment, the invention includes a kit for assessing a condition in a female mammal, comprising a sensor and at least one reagent.

In still other embodiments, this invention includes feminine hygiene products comprising a solid support and a substrate. The substrate includes a peptide that specifically reacts with a protein (e.g., a protein produced by a microorganism) and a calorimetric component coupled to the peptide. The peptide is coupled to the solid support. As used herein, a feminine hygiene product includes, a pad, a napkin, a liner, a swab, a wipe, and a tampon.

In still other embodiments, this invention includes consumer absorbent products comprising a solid support and a substrate. As used herein, a consumer absorbent product includes, diapers, pads, tampons and napkins.

As described herein, the present invention allows for the assessment of a condition in a female, including Bacterial Vaginosis (BV), Candidiasis (CV) and Trichomoniasis (TRIC). In all forms of vaginitis (e.g., BV, TRIC, CV) there is a strong correlation between protease secretion, damage to the epithelia (causing the characteristic formation of clue cells (vaginal epithelial cells coated with coccobacilli)), and infection. This invention can be used advantageously in a diverse range of roles, including providing utility in a healthcare setting or used as a point of care diagnostic. For example, in a healthcare setting, this invention will allow a user to assess and diagnose medical conditions so that the proper treatment can be prescribed. As another example of utility, this invention allows a human to conduct a preliminary assessment of a medical condition so that she can take a desired course of action (e.g., seeking medical care, conducting self-treatment, or modifying behavior to increase or decrease the chances of becoming pregnant).

The invention provides for a rapid and low cost assessment of a condition in a female mammal based on modification of a substrate that results in a visible signal, thereby eliminating the need for expensive equipment or additional medical supplies to assess the condition. The raw materials needed to practice this invention are inexpensive. Additionally, the methods and articles of this invention allow a portable means for assessing a medical condition, thereby eliminating the need to conduct detection or portions of detection in a laboratory or hospital setting.

In one embodiment, using labeled peptide libraries, specific and novel targets for Lactobacillus sp. and Gardnerella vaginalis that can be used independently or in combination to provide a simple, rapid, and very specific diagnostic for BV, have been identified. In another embodiment, using labeled peptide libraries, specific and novel targets for Candida spp. that can be used independently or in combination to provide a simple, rapid, and very specific diagnostic for CV, have been identified. In yet another embodiment, using labeled peptide libraries, specific and novel targets for Trichomonas vaginalis that can be used independently or in combination to provide a simple, rapid, and very specific diagnostic for TRIC, have been identified. All of these biomarkers are inexpensive and can be incorporated into a plethora of consumer products including, but not limited to, feminine napkins, wipes, pads, swabs and/or tampons.

In some embodiments, the invention includes a substrate comprising at least one colorimetric component attached to a peptide. In further embodiments, the substrate is attached to a solid support. In some embodiments, the peptide is one that will undergo a modification when it interacts with a protein. For example, the protein can be an enzyme and the modification can include the enzyme cleaving the peptide. In some embodiments, the peptide is synthetic, while in others, it is naturally occurring.

In some embodiments, the detectable signal includes a visual signal or a visible color change. In some embodiments, the visible signal includes a color change that is perceptible without any kind of detection equipment or enhancing equipment (e.g., a fluorometer), a change from one color or hue to another or to a colorless or less or more visible color or hue, and/or the initiation of a fluorescent or luminescent glow.

In some embodiments, the invention includes a substrate comprising at least one reporter enzyme (e.g., horseradish peroxidase) attached to a peptide, resulting in an enzyme-peptide conjugate. The modification can include cleavage by a different enzyme (for example, one produced by or associated with a microorganism, or marking a state, such as ovulation or bone activity). Upon cleaving the peptide, the detectable signal can result from the interaction between the reporter enzyme and its substrate.

In some embodiments, a signal amplification procedure can be used, for example, a procedure in which a reporter enzyme is conjugated with a specific peptide and hydrolysis leads to the activation of a catalytic process leading to a detectable signal (e.g., a visible color change).

In most embodiments, high throughput screening can be used to identify targets for microorganisms such as Gardnerella, Lactobacillus, Candida albicans, and Trichomonas vaginalis.

In one embodiment, the invention includes a method of assessing a condition in a female mammal, comprising: a) exposing an unmodified substrate to a sample under conditions that will result in a modification of the substrate, wherein the unmodified substrate includes a peptide and a first colorimetric component; and b) detecting the modification of the substrate or an absence of the modification of the substrate, wherein the modification comprises cleaving the first colorimetric component or enzyme from the substrate and results in a visible signal, wherein the modification or absence of the modification indicates a condition in the female mammal.

In one embodiment, a substrate of the invention is specific for a protein produced by Gardnerella vaginalis. In another embodiment, the substrate is specific for a protein produced by Lactobacillus spp. In yet another embodiment, the substrate is specific for a protein produced by Candida albicans. In one embodiment, the substrate is specific for a protein produced by Trichomonas vaginalis. In one embodiment, the substrate is specific for a protein produced by herpes simplex virus. In one embodiment, the substrate does not react with a protein produced by at least one microorganism selected from the group consisting of bacteria associated with BV such as Bacteriodes spp., Mobilincus spp., Peptostreptococcus spp., Mycoplasma hominis, Peptostreptococcus spp., Prevotella bivia, Porphyromonas spp., and Trichomonas spp. In yet another embodiment, the condition is at least one condition selected from the group consisting of candidiasis, trichomoniasis, bacterial vaginosis, a urinary tract infection, genital herpes, pre-ovulation, menopause, and osteoporosis.

In one embodiment, the invention further comprises measuring the pH of the sample. In another embodiment, the invention further comprises measuring the amount of volatile polyamines in the sample.

In yet another embodiment, the peptide is coupled to a solid support. In some embodiments, simple and inexpensive food grade dyes are conjugated to the peptides and coupled directly to the absorbent materials of, for example, a feminine napkin, a pad, a wipe or a tampon.

In yet another embodiment, in the presence of the specific microorganism, the peptide is hydrolyzed, allowing for the dye or enzyme to be collected at a surface, such as the bottom surface. By incorporating a transparent window at the surface of, for example, a pad or tampon, the color is visible from the surface. A color change in the window indicates the presence of vaginitis. The pad/tampon diagnostic device design can also be used for other analytes from vaginal fluid or urine to detect early onset of conditions, such as bacterial infections, microrganismal infections, urinary tract infections, yeast infections, genital herpes (HSV-2), pre-ovulation, menopause, or bone loss (osteoporosis).

In one embodiment, the modification of the substrate results in an increase in the visibility of the hue of the solid support. In another embodiment, the peptide is covalently attached to the solid support. In yet another embodiment, the solid support is selected from the group consisting of a bead, a sterilized material, an article that contains the sample, an article that collects the sample, a polymer, a membrane, a sponge, a disk, a scope, a filter, a foam, a cloth, a paper, a suture, and a bag.

In one embodiment, the solid support is incorporated in a product from the group consisting of a feminine napkin, a pad, a diaper, a wipe, a swab, and a tampon. In another embodiment, the first colorimetric component is a dye, such as a fluorescent dye, a luminescent dye or a chromogenic dye. In yet another embodiment, the first colorimetric component is an enzyme, such as horseradish peroxidase, a phenol oxidase, luciferase, galactosidase, laccase or alkaline phosphatase.

In one embodiment, the unmodified substrate further includes a second colorimetric component that is dissimilar to the first colorimetric component. In another embodiment, the first calorimetric component is covalently bonded to the peptide. In yet another embodiment, the modification includes hydrolysis of a peptide bond and results in a portion of the peptide detaching from the substrate.

In one embodiment, the substrate comprises a peptide comprising at least one or more amino acid sequence from the group consisting of: the peptide sequence PFINETYAKFC (SEQ ID NO: 1), the peptide sequence ITTTSSKHEHC (SEQ ID NO: 2), the peptide sequence KPKAFXXX (SEQ ID NO: 3), the peptide sequence VPGDPEAAEARRGQC (SEQ ID NO: 4), the peptide sequence KPKAFLKGRR (SEQ ID NO: 5), the peptide sequence KPKAFLKVGN (SEQ ID NO: 6), the peptide sequence LYPILKKNQK (SEQ ID NO: 7), the peptide sequence KPSIKPTPPY (SEQ ID NO: 8), the peptide sequence QKTTIKKLKH (SEQ ID NO: 9), the peptide sequence TPIQIHTILH (SEQ ID NO: 10), the peptide sequence INLSKKQIYP (SEQ ID NO: 11), the peptide sequence LYPSQNPVIK (SEQ ID NO: 12), the peptide sequence NITKKSTKII (SEQ ID NO: 13), the peptide sequence NNPLPKIQKN (SEQ ID NO: 14), the peptide sequence KNPKLQDHYI (SEQ ID NO: 15), the peptide sequence QINKALKQPK (SEQ ID NO: 16), the peptide sequence QIPKSLHPIT (SEQ ID NO: 17), the peptide sequence LHNYVLLRNIL (SEQ ID NO: 18), the peptide sequence SKQQDIIKKY (SEQ ID NO: 19), and the peptide sequence NKTNKTKHAY (SEQ ID NO: 20), the peptide sequence QRTTIRRLRH (SEQ ID NO: 21), and the peptide sequence ASNAEAGALVNASSAAHVDV (SEQ ID NO: 22).

In one embodiment, the visible signal includes an increase in a fluorescent glow or luminescent glow. In another embodiment, the visible signal includes a change in hue. In yet another embodiment, the visible signal is a loss of color.

In one embodiment, the sample includes a portion of a bodily fluid, including vaginal fluid or urine.

In one embodiment, the modification of the substrate includes cleaving a portion of the peptide to produce a cleaved portion, the cleaved portion comprising the first colorimetric component, the modification resulting in the migration of the cleaved portion toward a collector, and the migration resulting in a visible signal. In another embodiment, the collector includes at least one material selected from the group consisting: of a membrane, a particle, a bead, a resin, a polymer, a film, a gel and a chelating material.

In one embodiment, the modification of the substrate is used to indicate the presence of a bacterial enzyme selected from the group consisting of a lysin, an autolysin, a lipase, an exotoxin, a cell wall enzyme, a matrix binding enzyme, a protease, a hydrolase, a virulence factor enzyme, a hormone and a metabolic enzyme.

In another embodiment, the invention includes a sensor for detecting the presence or absence of a protein, comprising a peptide that specifically reacts with a protein produced by a microorganism and a first colorimetric component coupled to the peptide.

In another embodiment, the invention includes a feminine hygiene product, comprising a solid support and a substrate, wherein the substrate comprises a peptide that specifically reacts with a protein produced by a microorganism and a first colorimetric component or enzyme coupled to the peptide, and wherein the substrate is coupled to the solid support. In another embodiment, the feminine hygiene product is selected from the group consisting of a feminine napkin, a wipe, a swab, a pad and a tampon. In yet another embodiment, the feminine hygiene product further comprises at least one inner layer comprising absorbent material. In one embodiment, the substrate comprises a peptide specific for each of Gardnerella vaginalis, Trichomonas vaginalis and Candida albicans.

In one embodiment, the invention includes a method of detecting the presence or absence of an irritating factor in a mammal, wherein said irritating factor is selected from the group consisting of bacteria, yeast, parasites, protozoa, host proteases, and enzymes, and wherein said irritating factor is detected in an absorbent pad selected from the group consisting of a feminine napkin, pad and diaper, comprising: a) exposing an unmodified substrate to a sample under conditions that will result in a modification of the substrate, wherein the unmodified substrate includes a peptide and a first colorimetric component, wherein the first calorimetric component or is coupled to the peptide, and wherein the sample includes mammalian vaginal fluid or urine; and b) detecting the modification of the substrate or an absence of the modification of the substrate, wherein the modification comprises cleaving the first calorimetric component or enzyme from the substrate and results in a visible signal, wherein the modification or absence of the modification indicates presence or absence of an irritating factor in the mammal.

In another embodiment, the invention includes a method of detecting the presence or absence of a condition in a mammal, wherein said condition is selected from the group consisting of a urinary tract infection, yeast infection, bacterial vaginosis, candidiasis, trichomoniasis, skin rash, diaper rash and bed sore, comprising: a) exposing an unmodified substrate to a sample under conditions that will result in a modification of the substrate, wherein the unmodified substrate includes a peptide and a first colorimetric component, wherein the first calorimetric component or enzyme is coupled to the peptide, and wherein the sample includes mammalian vaginal fluid or urine; and b) detecting the modification of the substrate or an absence of the modification of the substrate, wherein the modification comprises cleaving the first calorimetric component from the substrate and results in a visible signal, wherein the modification or absence of the modification indicates presence or absence of an irritating factor in the mammal.

In yet another embodiment, the invention includes a method of assessing a condition in a mammal, comprising: a) exposing an unmodified substrate to a sample under conditions that will result in a modification of the substrate, wherein the unmodified substrate includes a peptide and a first colorimetric component, wherein the first colorimetric component or enzyme is coupled to the peptide; and b) detecting the modification of the substrate or an absence of the modification of the substrate, wherein the modification comprises cleaving the first colorimetric component from the substrate and results in a visible signal, wherein the modification or absence of the modification indicates a condition in the mammal.

In one embodiment, the invention includes a consumer absorbent product, comprising a solid support and a substrate, wherein the solid support has at least one absorbent layer of material that absorbs bodily fluid, and at least one non-absorbent layer to prevent leakage of the bodily fluid. In one embodiment, the bodily fluid is vaginal fluid. In another embodiment, the bodily fluid is urinary fluid.

In one embodiment, the first calorimetric component is a food grade dye or a reporter enzyme including any one of those described herein.

In another embodiment, the invention includes a lateral flow device for assessing a condition in a female mammal, wherein said lateral flow device comprises a lateral flow strip, a conjugate membrane, a substrate line and a wicking pad.

### BRIEF DESCRIPTION OF THE DRAWINGS

The foregoing and other objects, features and advantages of the invention will be apparent from the following more particular description of preferred embodiments of the invention, as illustrated in the accompanying drawings. The drawings are not necessarily to scale, emphasis instead being placed upon illustrating the principles of the invention.

FIG. 1 is a graph showing the UV/visible spectra in water of triazine dye Reactive Blue 4.

FIG. 2 is a graph showing the UV/visible spectra of triazine dye Reactive Yellow 86.

FIG. 3 is a graph showing the UV/visible spectra in water of REMAZOL® Brilliant Blue R.

FIG. 4 is a graph showing the UV/visible spectra of REMAZOL® Black B vinyl sulfone.

FIG. 5 is a drawing of one embodiment of the invention. The unmodified substrates comprises a peptide, a yellow colorimetric component, and a blue colorimetric component. The unmodified substrate has a green hue. After modification by a protease, a portion of the peptide that includes the yellow colorimetric component is cleaved, leaving the substrate with only a blue colorimetric component.

FIG. 6 is a drawing of a metal chelation embodiment of the invention. A colorimetric component, in this case a dye, is included on a peptide that is attached to a nickel-NTA resin solid support. A protease cleaves a portion of the peptide, and the cleaved portion has a greater affinity for a membrane collector than for the original surface. As the dye migrates towards the collector, the remaining peptide and solid support produce a visible color change.

FIG. 7 is a drawing of an embodiment of the invention where a charged membrane is coupled to a substrate that includes a colorimetric component. A protease cleaves a portion of the peptide, and the cleaved portion has a greater affinity for the membrane collector than for the original surface. As the dye migrates towards the collector, the remaining substrate and solid support produce a visible color change.

FIG. 8 is a drawing of construction of three peptide libraries using epitope tags (polyhistidine, FOlA), colorimetric components (horseradish peroxidase (HRP), green fluorescent protein (GFP), and lissamine rhodamine sulfonyl chloride (LRSC)), and sequences of 10 random amino acids (i.e., “Wobble” sequence, in which the nucleotide sequence at the gene level was randomized at each base).

FIG. 9 is a chart of the signal strength measured in the various wells of plate number 69.

FIG. 10 is a chart of the signal strength measured in the various wells of plate number 76.

FIG. 11 is a graph which illustrates the specificity of the GV2 (PFINETYAKFC (SEQ ID NO: 1)) peptide for Gardnerella vaginalis over the course of five minutes when incubated with Gardnerella vaginalis (“Gardnerella”), Staphylococcus aureus (“Staph A.”), Streptococcus pyogenes (“Strep P.”), Escherichia coli (E. coli), and Enterococcus faecalis (“Entero”).

FIG. 12 is a graph showing that the peptide ITTTSSKHEHC (SEQ ID NO: 2) detects an unidentified protease from Lactobacillus acidophilus (“Lacto”).

FIG. 13 is a drawing of one example of a design for a feminine napkin.

FIG. 14 is a scan of a gel electrophoresis of Kanamycin peptide library clones.

FIG. 15A-FIG. 15B is a list of pH indicators.

FIG. 16 is a drawing of an enzyme sensor. Immobile polystyrene latex beads (not to scale) are functionalized with peptide-HRP conjugates. In the presence of specific microbial proteases, the peptide sequences are clipped, allowing the HRP to leave the bead surface. In the presence of hydrogen peroxide, a colorimetric substrate for HRP will change color, giving a detectable signal of pathogens.

FIG. 17 is a drawing of a dye based sensor. A side-view of a section of the sensor is presented. A dye-peptide conjugate is covalently attached to a membrane (A). Initially, the blue color will not be visible to the user, as it will be covered by a second white membrane with a high affinity for the dye, such as ICE (Pall) (B). The presence of specific microbial proteases will liberate dye molecules via proteolytic cleavage of the peptide sequence. These dye molecules will then be free to migrate to the top surface of the dye capture membrane (C) and generate a visible blue signal indicating the presence of high levels of microbial pathogens.

FIG. 18 illustrates PCR primers used in quantitative PCR (qPCR) to validate diagnostic sensors and confirm the levels of each pathogen from clinical vaginal swab samples. The variable regions of the rDNA were used to determine primers for C. albicans, E. coli. and L. acidophilus that would not cross-react with one another but would recognize the organism of interest. The variable regions of the rDNA from each organism were aligned and this was used as a guideline for primer design. The alignment with the variable (VI) region is highlighted. The top row indicates the contiguous amino acid sequence.

FIG. 19 is a scan of a gel with PCR products that are the positive controls for each ribosomal PCR product. The rDNA primers for E. coli (E), C. albicans (C), G. vaginalis and L. acidophilus (L) are specific. Gel lanes contain the following samples for each gel. Lane 1: C. albicans, Lane 2: G. vaginalis; Lane 3: L. acidophilus, Lane 4: E. coli, Lane 5: No template.

FIG. 20 is a lateral flow diagnostic with the control (left) containing no bacteria and the test strip (right) containing bacteria. The Naphthol line on the strip with the bacteria turned purple, thus yielding a positive reading for the presence of bacteria.

FIG. 21 is a picture of the inner materials of a point of care lateral flow device. The device has four components: a lateral flow strip (1), a conjugate membrane (2), a substrate line (3), and a wicking pad (4).

FIG. 22 is a graph showing that the peptide (GV2) PFINETYAKFC (SEQ ID NO: 1) detects unidentified protease from G. vaginalis.

FIG. 23 is a plot graph of a specific peptide (G11, which is TPIQIHTILH (SEQ ID NO: 10)), which is able to detect Candida albicans.

FIG. 24 is a table of C. albicans clinical vaginal isolates for target peptides (H2 (QKTTIKKLKH (SEQ ID NO: 9)) and R8 (KPKAFLKVGN (SEQ ID NO: 6)).

### DETAILED DESCRIPTION OF THE INVENTION

A description of preferred embodiments of the invention follows.

While this invention has been particularly shown and described with references to preferred embodiments thereof, it will be understood by those skilled in the art that various changes in form and details may be made therein without departing from the scope of the invention encompassed by the appended claims.

The present invention encompasses compositions and methods useful for the assessment of a condition in a female mammal. This invention includes a substrate, typically a protein or peptide, comprising a calorimetric component that produces a detectable signal that indicates when the substrate has undergone a modification. The modification can be the result of the substrate contacting a protein, such as an enzyme (for example, an enzyme associated with or produced by a microorganism), resulting in the modification of the substrate by, for example, enzymatic cleavage. The detectable signal is used to assess a condition in a female mammal.

In some embodiments, for example, detection of diaper rash, the mammal can be male or female.

Enzyme Detection

As part of their normal growth processes, many microorganisms secrete, or cause to be secreted, a number of proteins into their growth environment, such as enzymes. These proteins have numerous functions including, but not limited to, the release of nutrients, protection against host defenses, cell envelope synthesis (in bacteria) and/or maintenance, and others as yet undetermined. Many microorganisms also produce proteins on their cell surface that are exposed to (and interact with) the extracellular environment. Many of these proteins are specific to the microorganism that secretes them, and as such, can serve as specific markers for the presence of those microorganisms, which in turn provides for assessment of medical conditions. This invention includes a method and/or system that can detect the presence of these produced and/or secreted proteins and can equally serve to indicate the presence of the producing/secreting microorganism. Alternatively, this invention provides for a method and/or system that can detect the absence of these produced and/or secreted proteins so as to indicate the absence of the producing/secreting microorganism. Such a detection method and/or system are useful for detecting or diagnosing the presence of a microorganism and/or an infection (e.g., a vaginal or urinary fluid infection or a tissue infection).

In some embodiments, a microorganism produces the protein that modifies the substrate. This invention includes methods for detecting the presence or absence of a microorganism in a sample in order to assess a medical condition in a female mammal. For example, the method can comprise the steps of a) contacting the sample with a substrate under conditions that will result in a modification of the substrate by the microorganism and b) detecting the modification or an absence of the modification. A protein produced, secreted, or expressed by the microorganism modifies the substrate. In some embodiments, the modification comprises cleaving at least a portion of the substrate, wherein the portion includes one of the colorimetric components and the cleaving results in a visible color change, thus indicating the presence of the microorganism in the sample, and absence of the modification indicates the absence of the microorganism.

In other embodiments, the invention includes a method for detecting the presence or absence of an enzyme in a sample. In one example, the method comprises the steps of a) contacting the sample with a substrate under conditions that will result in a modification of the substrate by the enzyme and b) detecting the modification or an absence of the modification. In some embodiments, the modification comprises hydrolyzing at least one peptide bond in the peptide and resulting in at least a portion of the peptide being cleaved from the substrate, wherein the portion of the peptide cleaved from the substrate includes one of the calorimetric components and wherein the cleaving results in a visible color change, thus indicating the presence of the enzyme in the sample, and absence of the modification indicates the absence of the enzyme in the sample.

A sensor, as described herein, can be tailored to detect one specific microorganism by identifying a protein, such as a secreted enzyme, specific to the microorganism to be detected. Alternatively, a system can be designed to simultaneously identify more than one microorganism species (for example, at least 2, at least 5, or at least 10 different microorganism species), such as those associated with vaginitis. Preferably, this goal is achieved by identifying those proteins that are common to certain classes of pathogenic microorganisms, but which are not common to non-pathogenic microorganisms. Such proteins can be identified, for example, with a computer based bioinformatics screen of the microbial genomic databases.

The presence of a microorganism can be detected by designing a synthetic substrate that will specifically react with a protein that is present on the surface of the cell or is secreted into the microorganism's growth environment. These synthetic substrates can be labeled with a detectable label such that under conditions wherein their respective proteins specifically react with them, the substrates undergo a modification that is indicated by the detectable label, e.g., a calorimetric component. The detectable label can produce a visible color change.

Examples of microorganisms that can be detected by the various embodiments of this invention include bacteria, viruses, fungi, parasites, protozoa, and other pathogens, thereby allowing for the assessment of a medical condition in a female mammal. In some embodiments, the microorganism is a bacterium.

In some embodiments, this invention can be used to detect one or more types of fungi, thereby allowing for the assessment of a medical condition in a female mammal. Preferably the fungi are those that cause disease in female mammals or are useful as indicators of medical conditions in female mammals.

In some embodiments, the protein that interacts with the substrate to produce the modification is an enzyme. Since a small amount of enzyme can catalyze the turnover of a substantial amount of substrate, basing a detection system on enzymes provides for sensitive tests. In other embodiments, the proteins are pathogen-specific enzymes. As used herein, a “pathogen-specific enzyme” is an enzyme produced and/or secreted by a pathogenic microorganism, but not produced and/or secreted by a non-pathogenic microorganism.

Enzymes can be grouped into classes insofar as they represent targets for developing agents to detect the microorganisms or female conditions that produce them and present them on the cell surface or secrete them into their growth environment. As described herein, enzymes are grouped into nine classes: a lysin (i.e., an enzyme that functions to lyse host cells), an autolysin, an exotoxin, a matrix binding enzyme, a lipase; a cell wall enzyme (i.e., an enzyme involved in the synthesis and turnover of bacterial cell wall components, including peptidoglycan), a protease (i.e., an enzyme that specifically or non-specifically cleaves a peptide, polypeptide, or protein), a hydrolase (i.e., an enzyme that breaks down polymeric molecules into their subunits), a metabolic enzyme (i.e., an enzyme designed to perform various housekeeping functions of the cell, such as breaking down nutrients into components that are useful to the cell), a virulence factor enzyme (i.e., an enzyme that is required by the bacterial cell to cause an infection). Enzymes can also represent targets for developing agents to detect a hormone, such as a menstrual hormone or a hormone of the reproductive system (such as, estrogen, progesterone and luteinizing hormone). Such enzymes would be useful, for example, for detecting ovulation.

Conditions

The various embodiments of this invention can be used to assess conditions, including medical conditions or diseases, in a female mammal (e.g., a human female). For example, the methods, substrates, sensors, and other aspects of this invention can be used to detect the presence of one or more specific types of microorganisms and thereby assess whether a female mammal has a medical condition, such as vaginitis or a female lower genital tract infection.

On one embodiment, is a method of detecting the presence or absence of a an condition in a mammal, wherein said condition is selected from the group consisting of a urinary tract infection, yeast infection, bacterial vaginosis, candidiasis, trichomoniasis, skin rash, diaper rash and bed sore.

The major bacteria associated with diagnosing BV is the gram-positive rod shaped bacterium Gardnerella vaginalis. See Catlin, B. W., “Gardnerella Vaginalis: Characteristics, Clinical Considerations, and Controversies,” Clin Microbiol Rev, 213-237 (1992)). However, other bacteria have been recently implicated in colonizing following the onset of BV including Bacteriodes spp., Mobilincus spp., Peptostreptococcus spp., Mycoplasma hominis, Prevotella bivia, and Porphyromonas spp. See Hogan, D. A. et al., “Pseudomonas-Candida Interactions: An Ecological Role for Virulence Factors,” Science, 296:2229-2231 (2002) and Marrazzo, J. M., “Evolving Issues in Understanding and Treating Bacterial Vaginosis,” Expert Rev Anti Infect Ther., 2(6):913-22 (2004).

Major BV indicators include the Amsel criterion and the Nugent test. See Carlson, P. et al., “Evaluation of the Oricult-N Dipslide for Laboratory Diagnosis of Vaginal Candidiasis,” J Clin Microbiol, 32(3):1063-1065 (2000), Sobel, J. D. et al., “Vulvovaginal Candidiasis: Epidemiologic, Diagnostic, and Therapeutic Considerations,” Am J Obstet Gynecol, 1 78:203-211 (1998), Altschul, S. F. et al., “Gapped BLAST and PSIBLAST: a New Generation of Protein Database Search Programs,” Nucleic Acids Res, 25:2289-3402 (1997), Amundson, N. R. et al., “DNA Macrorestriction Analysis of Nontypeable Group B Streptococcal Isolates: Clonal Evolution of Nontypeable and Type V Isolates,” J Clin Microbiol, 572-576 (2005), Nugent, R. P. et al., “Reliability of Diagnosing Bacterial Vaginosis is Improved by a Standardized Method of Gram Stain Interpretation,” J Clin Microbiol, 29:297-301 (1991), and Landers, D. V. et al., “Predictive Value of the Clinical Diagnosis of Lower Genital Tract Infection in Women,” Am J Obstet Gynecol, 190:1004-10 (2004). Other indicators for diagnosing BV include a decrease in normal bacterial flora (such as Lactobacillus sp.), a decrease in lactate, an increase in pH above 4.5, and the production of volatile polyamines. See, for example, U.S. Patent Application Publication No. U.S. 2003/0044996, U.S. Pat. No. 6,113,856, and U.S. Pat. No. 5,124,254, the teachings of which are all incorporated herein by reference. See also Donders, G. G. et al., “Pathogenesis of Abnormal Vaginal Bacterial Flora,” Am J Obstet Gynecol, 182:872-878 (2000), Antonio, M. et al., “DNA Fingerprinting of Lactobacillus Crispatus Strain CTV-05 by Repetitive Element Sequence-Based PCR Analysis in a Pilot Study of Vaginal Colonization,” J Clin Microbiol, 1881-1887 (2003), and Geshnizgani, A. M. et al., “Defined Medium Simulating Genital Tract Secretions for Growth of Vaginal Microflora,” J Clin Microbiol, 30:1323-1326 (1992). However, individual indicators can provide false positives due to such occurrences as Trichomoniasis (TRIC), group B Streptococcus, normal pH changes during menses, the reduction of Lactobacilli as women approach post-menopausal age, and changes in polyamines due to cervical cancer. See Bradshaw, C. S. et al., “Evaluation of a Point-of-Care Test, BVBlue, and Clinical and Laboratory Criteria for Diagnois of Bacterial Vaginosis,” J Clin Microbiol 1304-1308 (2005), Myziuk, L. et al., “BVBlue Test for Diagnosis of Bacterial Vaginosis,” J Clin Microbiol, 41(5): 1925-1928 (2003).and Tokyol, C. et al., “Bacterial Vaginosis: Comparison of Pap Smear and Microbiological Test Results,” Mod Pathol., 17(7):857-60 (2004).

Additional tests for BV include sialidase, prolidase, and chrome azurol S assays that detect the presence of Gardnerella vaginalis. See, for example, U.S. Patent Application Publication No. U.S. 2004/0219617 and U.S. Pat. No. 6,255,066. See also Brown, H. L. et al., “Evaluation of the Affirm Ambient Temperature Transport System for the Detection and Identification of Trichomonas Vaginalis, Gardnerella Vaginalis, and Candida Species from Vaginal Fluid Specimens,” J Clin Microbiol, 3197-3199 (2001) and Madico, G. et al., “Diagnosis of Trichomonas Vaginalis Infection by PCR Using Vaginal Swab Samples,” J Clin Microbiol, 36:3205-3210 (1998). However, these tests are not specific for Gardnerella vaginalis and can lead to the presence of false positives due to interference with other microbes, yeast, Trichomonas vaginalis, and/or atypical vaginal cells. See Sheiness, D. et al., “High Levels of Gardnerella Vaginalis Detected With an Oligonucleotide Probe Combined With Elevated pH as a Diagnostic Indicator or Bacterial Vaginosis,” J Clin Microbiol, 30:642-648 (1992) and Wu, S. R. et al., “Genomic DNA Fingerprint Analysis of Biotype 1 Gardnerella Vaginalis From Patients With and Without Bacterial Vaginosis,” J Clin Microbiol, 192-195 (1996). Further, there is increasing resistance to topical treatments such as clindamycin and metronidazole.

Candida albicans is the leading pathogen associated with yeast infections, although other Candida species appear to be emerging. In particular, C. glabrata represents as much as 25% of the yeast infections and is more commonly found in patients with diabetes. See McDonald, H. et al., “Antibiotics for Treating Bacterial Vaginosis in Pregnancy,” Cochrane Database Syst Rev., (1) (2005). Both BV and CV are associated with a decrease in Lactobacillus; whereas BV is associated with an increase in pH, CV is associated with a normal pH.

Recent studies indicate that toxins and H₂O₂ produced by Lactobacillus are able to inhibit growth of Candida spp. See Reid, G. et al., “Nucleic Acid-Based Diagnosis of Bacterial Vaginosis and Improved Management Using Probiotic Lactobacilli,” J Med Food., 7(2):223-8 (2004). CV develops clinically into a discharge containing epithelial cells, hyphae, and pseudohyphae. See Fidel, P. L. et al., “Effects of Reproductive Hormones on Experimental Vaginal Candidiasis,” Infect Immun, 651-657 (2000). If not treated, over time a secondary infection may develop in the urethra. Although yeast infections can be treated over-the-counter (OTC) without a prescription with products such as MONOSTA™, there are no OTC self-monitoring diagnostic tests for yeast infections. See Nelson, D. B. et al., “Self-Collected Versus Provider-Collected Vaginal Swabs for the Diagnosis of Bacterial Vaginosis: an Assessment of Validity and Reliability,” J Clin Epidemiol, 56:862-866 (2003). It is predicted that seventy-five percent of all women experience at least one episode of CV and of BV during their lifetime. Gardnerella vaginalis can be detected using a substrate with the peptide sequence PFINETYAKFC (SEQ ID NO: 1), the peptide sequence LYPILKKNQK (SEQ ID NO: 7) and/or a modified peptide, for example, one containing one or more conserved amino acid substitutions.

TRIC is caused by Trichomonas vaginalis, a motile pear-shaped protozoa. Trichomonas vaginalis can ingest bacteria and blood cells and has proteases and toxins on its surface that mediate epithelial cell damage. The infectious dose is approximately ˜10⁵ protists and the symptoms include vaginal discharge, vulvovaginal soreness or irritation, dysuria (pain or difficulty urinating), and dyspareunia (pain during intercourse). See Lehker, M. W. et al., “Trichomonad Invasion of the Mucous Layer Requires Adhesins, Mucinases, and Motility,” Sex Transm Dis, 75:231-238 (1999) and Min, D. Y. et al., “Degradation of Human Immunoglobulins and Cytotoxicity on HeLa Cells be Live Trichomonas Vaginalis,” Korean J Parasitology, 35:39-46 (1997). The complications include vaginitis emphysematosa, infertility and complications in pregnancy (spontaneous abortions, preterm birth, and low birth weight). See Ness, R. B. et al., “Bacterial Vaginosis and Risk of Pelvic Inflammatory Disease,” Obstet Gynecol Surv., 60(2): 99-100 (2005) and Ness, R. B. et al., “Bacterial Vaginosis and Risk of Pelvic Inflammatory Disease,” Obstet Gynecol., 104(4):761-9 (2004). TRIC is often misdiagnosed as BV because of the lack of a reliable test. See Zariffard, M. R. et al., “Detection of Bacterial Vaginosis-Related Organisms by Real-Time PCR for Lactobacilli, Gardnerella Vaginalis and Mycoplasma Hominis,” FEMS Immunol Med Microbiol., 34(4):277-81 (2002). Both BV and TRIC can result in vaginal discharge and increase in pH. TRIC is most often identified using microscope examination. Metronidazole is used to kill Trichomonas vaginalis, but side effects of the drug include nausea and reduction of the levels of good bacteria in the vaginal fluids. Long term doses of metronidazole have been shown to cause lung tumors in animals, and many strains of Trichomonas vaginalis have become resistant to this drug. Trichomonas vaginalis can be detected using a substrate with the peptide sequence NNPLPKIQKN (38H9/T7) (SEQ ID NO: 14), the peptide sequence KNPKLQDHYI (44B5/T7) (SEQ ID NO: 15), the peptide sequence QINKALKQPK (41 El 1/T7) (SEQ ID NO: 16), the peptide sequence QIPKSLHPIT (42D3/T7) (SEQ ID NO: 17), the peptide sequence LHNYVLLRNIL (38H8/T7) (SEQ ID NO: 18), the peptide sequence SKQQDIIKKY (44E6/T7) (SEQ ID NO: 19), the peptide sequence NKTNKTKHAY (42H8/T7) (SEQ ID NO: 20) and/or a modified peptide, for example, one containing one or more conserved amino acid substitutions.

For example, the methods, substrates, sensors, and other articles of this invention can be used to determine if a female has a critical infection level of Gardnerella vaginalis (e.g., about 10⁵ to about 10⁶ CFU/mL), infectious filamentous Candida albicans (e.g., about 10⁶ CFU/mL), and/or Trichomonas vaginalis (e.g., ≧1000 protists/mL) in her vaginal and/or urinary tract. For example, the invention will produce a visible signal that indicates to the practitioner whether such microorganisms are present, for assessment of whether the female has the medical condition of bacterial vaginosis. Alternatively, or additionally, the invention can be used to assess other indicators of bacterial vaginosis, such as whether there is an abnormally low number or amount of normal bacteria in her vaginal or urinary tract (e.g., Lactobacillus sp.).

Other conditions or states that can be assessed include for example, whether the female is suffering from the early onset of a yeast infection by, for example, detecting the presence and/or amount of Candida albicans in the female's vaginal or urinary tract, whether the female is suffering from genital herpes (by, for example, detecting the presence and/or amount of HSV-2 in the female's vaginal or urinary tract); whether the female is in a pre-ovulation stage of her menstrual cycle; whether the female is going through menopause; or whether the female is experiencing bone loss (e.g., detecting or diagnosing osteoporosis).

Yeast infections can be ascertained from factors secreted by yeast into the urine or vaginal fluid, such as one of the secreted aspartate proteinases (Saps), lipase or other virulence factors. For example, a peptide marker made to the peptide KPKAFXXX (SEQ ID NO: 3), KPKAFLKXXX (SEQ ID NO: 24) or KPKAFXXXXX (SEQ ID NO: 23) (where “X” is any amino acid residue) is expected to be recognized by Saps from Candida albicans but not by host protease or bacteria in the vaginal fluids. CV is an opportunistic infection by Candida spp. that often can become pathogenic and cause infections. Candida spp. are dimorphic. They have a yeast form and a filamentous form. The latter is infectious. The leading determinants of infection onset appear to be hyphal growth and the production of a family of aspartyl proteases (Saps). Sap5 likely accelerates infection by causing necrosis and damage to the surrounding tissue, thereby making the environment more favorable to hyphal growth and infection. Candida spp. can be detected using a substrate with the peptide sequence KPKAFXXX (SEQ ID NO: 3), the peptide sequence KPKAFLKGRR (SEQ ID NO: 5), the peptide sequence KPKAFLKVGN (SEQ ID NO: 6), the peptide sequence KPSIKPTPPY (SEQ ID NO: 8), the peptide sequence QKTTIKKLKH (SEQ ID NO: 9), the peptide sequence TPIQIHTILH (SEQ ID NO: 10), the peptide sequence INLSKKQIYP (SEQ ID NO: 11), the peptide sequence LYPSQNPVIK (SEQ ID NO: 12), and the peptide sequence NITKKSTKII (SEQ ID NO: 13), and the peptide sequence QRTTIRRLRH (SEQ ID NO: 21), the peptide sequence KPKAFLKXXX (SEQ ID NO: 24), the peptide sequence KPKAFXXXXX (SEQ ID NO: 23) and/or a modified peptide, for example, one containing one or more conserved amino acid substitutions.

Targets specific to urinary tract infections (UTIs) can be optimally designed, for example, by identifying a broad spectrum peptide from a high throughput screen that would detect the major urinary tract pathogens including, for example, E. coli, K pneumoniae, P. mirabalis, P. aeruginosa, and Enterobacter spp. The prevalence of each in a UTI is: E. coli (˜37%), K pneumoniae (˜13%), P. mirabalis (˜12%), P. aeruginosa (˜9%), and Enterobacter spp. (˜7%).

Pre-ovulation can be measured precisely within about an hour by enzymes that are activated or over-expressed at the time of follicle rupture such as, for example, disintegrin (ADAM-TS1), MMPs (e.g., 1, 2, 9 and 13), ornithine decarboxylase (ODC), cathepsins, procathepsin-L, plasmin, or cyclooxygenase (COX-2). ADAM-TS 1 can be cloned from gingival fibroblasts and expressed and purified, and can be detected with the sequence VPGDPEAAEARRGQC (SEQ ID NO: 4) or a related brevican/versican target sequence. The cysteine on the C-terminus of the polypeptide can be used for coupling a dye or enzyme.

Substrates for matrix metalloproteinases (MPs) include, but are not limited to, collagen, gelatin, aggrecan and perlecan. Substrates for disintegrin include, but are not limited to, versican and brevican. Substrates for cathepsins include, but are not limited to, fibronectin, collagen, elastin, laminin, insulin B chain, and procathepsin/GAG. Substrates for COX-2 include, but are not limited to, amplex red. Substrates for ornithine decarboxylase (ODC) include, but are not limited to, ornithine. Substrates for plasmin include, but are not limited, to fibrin.

In one embodiment, fluorogenic and chromogenic substrates can be designed to specifically measure the activities of the enzymes identified above as potential pre-ovulatory markers. The following can be used: fluorescence resonance energy transfer (FRET) peptides to the insulin B chain, versican, and fibrin to detect procathepsin-L, ADAM-TS1, and plasmin respectively. MMP and COX2 substrates can be obtained from R&D Systems (Minneapolis, Minn.) and Molecular Probes (Eugene, Oreg.). The ODC substrate can consist of a pH indicator probe in the presence of ornithine to indirectly measure the decarboxylase activity.

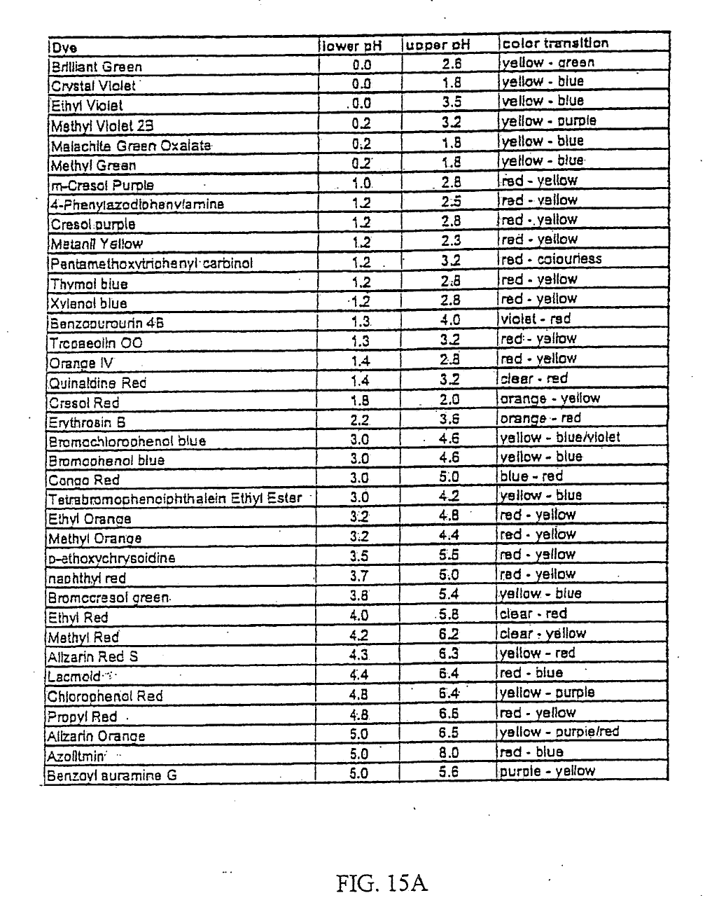

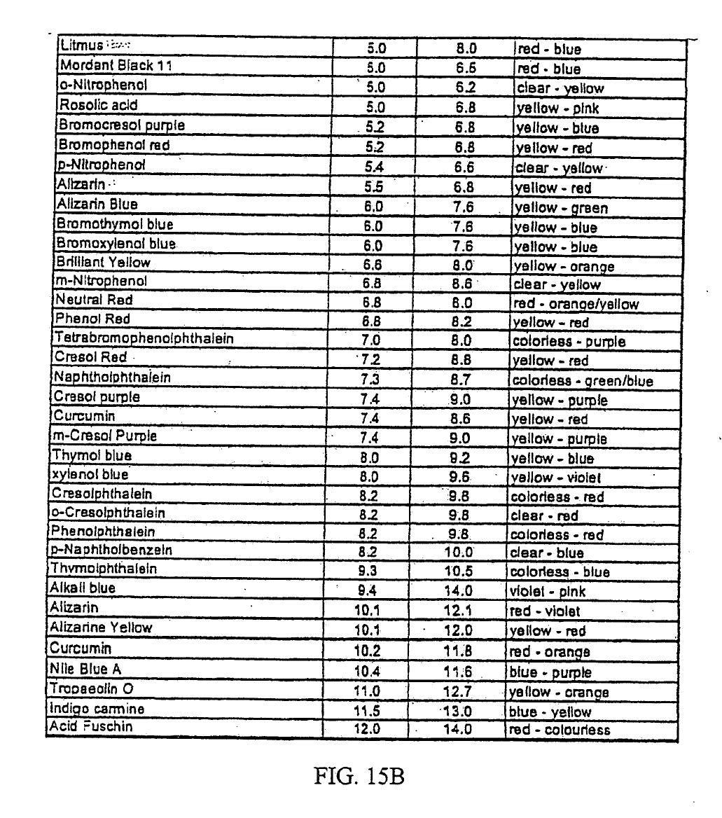

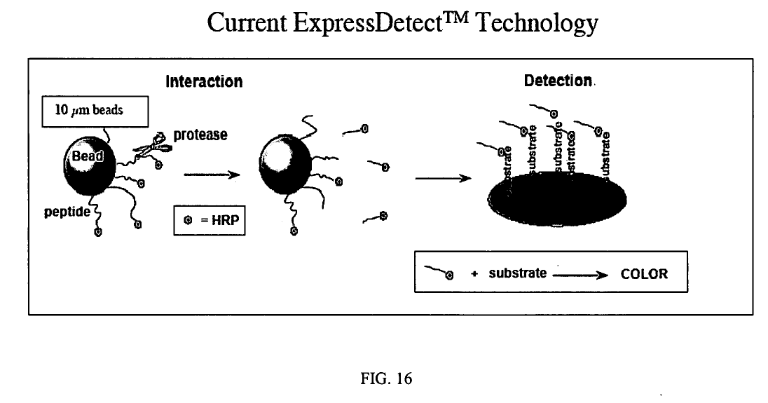

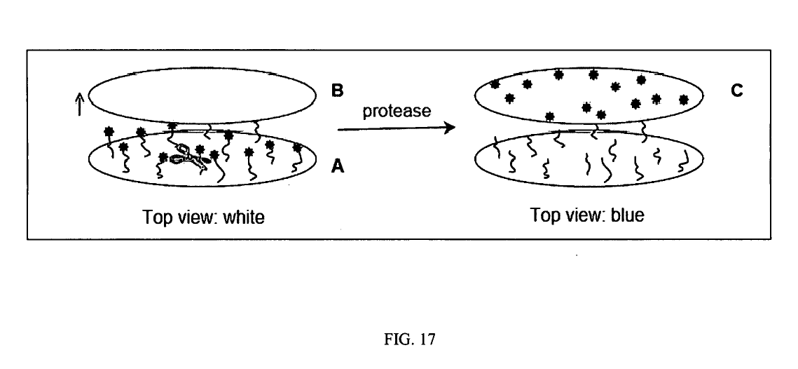

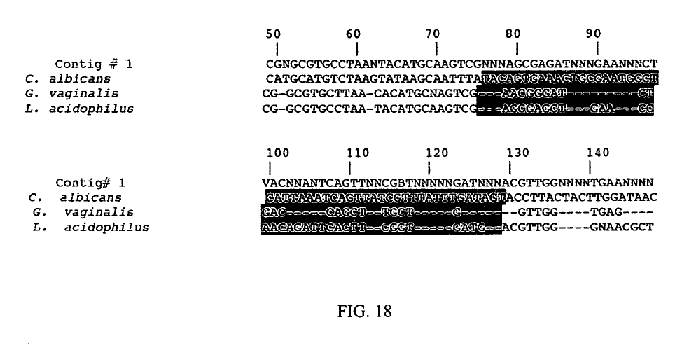

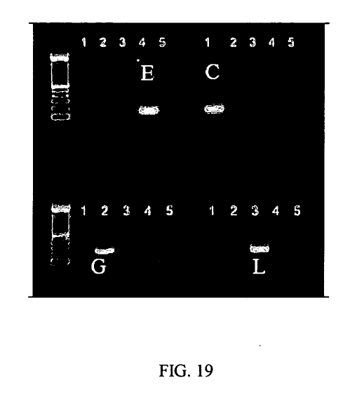

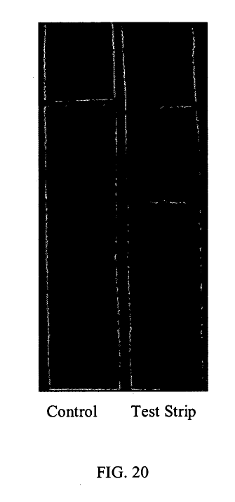

Examples of pH indicators for detecting pH levels include, but are not limited to, lacmoid, chlorophenol red, propyl red and bromcresol green. A list of additional pH indicators is provided in FIGS. 15A-15B.

Purified MMP enzymes can be obtained from, for example, R&D Systems. ODC and COX2 can be purchased from, for example, Sigma Aldrich. Procathepsin-L can be obtained from Calbiochem. ADAMTS-1 can be cloned by, for example, RT-PCR from human heart tissue. The protein will be expressed in COS-7 cells and purified from the culture media. The enzyme activity can be measured in a fluorescent or colorometric plate reader to quantify the sensitivity and cross reactivity with and without human fluid samples. The preovulatory targets that have the highest sensitivity and signal-to-noise ratio can be developed further to incorporate the chemistries into a wipe or pad.

Menopause onset has been correlated with the loss of activin B from saliva. Examples of bio markers for bone loss include calcium, procollagen type I C-terminal telopeptide (ICTP), and serum C-telopeptide of type I collagen, but none of them directly reflect the balance of building bone with osteoblasts and loss of bone with osteoclasts. More reliable markers can measure bone loss or bone gain (such as, for example, alkaline phosphatases and osteocalcin). A marker can measure the equilibrium between bone loss and bone gain. In one embodiment of the invention, for example, a bone loss marker with a blue reporter or dye and a bone gain marker with a yellow reporter or dye produces a green signature color if there is no net gain or loss of bone, a blue color if there is a net bone loss (which would be the first direct biochemical measurement of osteoporosis), or a yellow color if bone is being formed.

In another embodiment, the substrates, sensors, and methods used herein can be used to detect skin rashes mediated by infection, such as, diaper rash in male and female mammals. Diaper rash involves inflammation of the skin. It is a type of dermatitis caused by irritants, and is usually localized to the diaper area. It can be caused by infection by microorganisms including, for example, bacteria, fungi and yeast.

In one embodiment, early stages of diaper irritation or rash can be detected, even before they are visually detectable. A number of microorganisms are implicated in diaper rash, including lytic enzymes that may be present with a skin irritation or rash, such as, MMPs or other hydrolytic enzymes from the host or a microorganism, including MMPs, hydrolytic enzymes, lipases, sugarases, elastase and proteases.

In one embodiment of the invention, the microrganisms can be grown overnight in their respective media, also known as microbial supernatant, and then centrifuged to remove the cells. Five μl of each supernatant can be incubated with a peptide library for 20 minutes at room temperature. The peptide activity can be measured by the release of green fluorescent protein (GFP) from nickel-nitrilotriacetic resin (nickel-NTA resin) treated plates. The clones of the peptide conjugate targets that show promise as specific reporters for the microorganisms can be sequenced by DNA sequencing. The identified primary sequence of the positive clones can be used to generate fluorescence resonance energy transfer (FRET) and chromogenic peptides that can be incorporated into a membrane format. The amino acid sequence can be used to convert the validated peptides into enzyme-peptide or color dye-peptide conjugates which can be incorporated into a device.

Peptides

Examples of peptides include those substrates described herein, as well as those peptides known in the art to undergo modification by interaction with a protein. For example, U.S. patent application Ser. No. 11/036,761, filed Jan. 14, 2005, by Mitchell C. Sanders, entitled A Device for Detecting Bacterial Contamination and Method of Use; U.S. patent application Ser. No. 10/502,882, which is the U.S. National Stage of International Application Number PCT/US03/03172 filed on Jan. 31, 2003, and International Application Number PCT/US2004/036469 filed on Nov. 3, 2004, describe such peptides and their teachings are incorporated herein by reference in their entirety.

Examples of suitable peptides include peptides comprising or consisting of the sequence PFINETYAKFC (referred to herein as SEQ ID NO: 1), the sequence ITTTSSKHEHC (referred to herein as SEQ ID NO: 2), the sequence KPKAFXXX (referred to herein as SEQ ID NO: 3), the sequence VPGDPEAAEARRGQC (referred to herein as SEQ ID NO: 4), the sequence KPKAFLKGRR (referred to herein as SEQ ID NO: 5), the sequence KPKAFLKVGN (referred to herein as SEQ ID NO: 6), the sequence LYPILKKNQK (referred to herein as SEQ ID NO: 7), the sequence KPSIKPTPPY (referred to herein as SEQ ID NO: 8), the sequence QKTTIKKLKH (referred to herein as SEQ ID NO: 9), the sequence TPIQIHTILH (referred to herein as SEQ ID NO: 10), the sequence INLSKKQIYP (referred to herein as SEQ ID NO: 11), the sequence LYPSQNPVIK (referred to herein as SEQ ID NO: 12), and the sequence NITKKSTKII (referred to herein as SEQ ID NO: 13), and the sequence NNPLPKIQKN (referred to herein as SEQ ID NO: 14), and the sequence KNPKLQDHYI (referred to herein as SEQ ID NO: 15), and the sequence QINKALKQPK (referred to herein as SEQ ID NO: 16), and the sequence QIPKSLHPIT (referred to herein as SEQ ID NO: 17), and the sequence LHNYVLLRNIL (referred to herein as SEQ ID NO: 18), and the sequence SKQQDIIKKY (referred to herein as SEQ ID NO: 19), and the sequence NKTNKTKHAY (referred to herein as SEQ ID NO: 20), the sequence QRTTIKKLKH (referred to herein as SEQ ID NO: 21), the sequence ASNAEAGALVNASSAAHVDV (referred to herein as SEQ ID NO: 22) and /or a modified peptide, for example, one containing one or more conserved amino acid substitutions, and peptides that incorporate or comprise SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID 5 NO: 10, SEQ ID NO: 1, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 and/or SEQ ID NO: 22 or a modified peptide described herein. In regard to these sequences, amino acid groups such as cysteine can be added to the sequence to allow attachment of dyes or enzymes used as reporters.

Such substrates described herein can be obtained from commercial sources, such as Sigma-Aldrich, Corp. (St. Louis, Mo.), Molecular Probes (Eugene, Oreg.), New England Peptide (Gardner, Mass.) or can be produced (e.g., isolated, purified, or synthesized) using methods known to those of skill in the art.

In some embodiments, the peptides hybridize to the complement of an amino acid sequence described herein, for example, under conditions of high specificity, as known in the art. (See, e.g., Ausubel, F. M. et al. (“Current Protocols in Molecular Biology”, John Wiley & Sons, vol. 1 (1998).

In some embodiments, additional side groups are attached to one of the amino acids of the peptide chain. For example, a substrate of the invention can include a benzyl ether protecting group bound to one or more of the serine acids on the peptide chain. Protecting groups are chemical groups that are used to protect an amino acid from reacting with a colorimetric component. The use of protecting groups allows for labeling of the same type of amino acid in one peptide with two colorimetric components. For example, one serine group in a peptide can be protected with a benzyl ether group and a second serine, which is not protected, can be reacted with one colorimetric component. The protecting group can be removed and the second serine can be reacted with a different color component, thereby creating a substrate with two different color components.

The peptides of the invention also encompass fragments and sequence variants of the peptides described herein. Variants include a substantially homologous peptide encoded by the same genetic locus in an organism, i.e., an allelic variant, as well as other variants. Variants also encompass peptides derived from other genetic loci in an organism. Variants also include peptides substantially homologous or identical to these peptides but derived from another organism and/or d and l isomers (i.e., an ortholog), produced by chemical synthesis, or produced by recombinant methods.

In some embodiments, the peptide that undergoes modification through interaction with a protein comprises an amino acid sequence, such as one of the sequences listed herein or a sequence having at least 50%, 60%, 70%, 80%, 85%, 90%, 95%, or 99% sequence identity to one of the sequences listed herein, as determined using a sequence comparison program and parameters described herein.

The percent identity of two amino acid sequences can be determined by aligning the sequences for optimal comparison purposes (e.g., gaps can be introduced in the sequence of a first sequence). The amino acids at corresponding positions are then compared, and the percent identity between the two sequences is a function of the number of identical positions shared by the sequences (i.e., % identity=# of identical positions/total # of positions×100). In certain embodiments, the length of the amino acid sequence aligned for comparison purposes is at least 30%, preferably, at least 40%, more preferably, at least 60%, and even more preferably, at least 70%, 80%, 90%, or 100% of the length of the reference sequence. The actual comparison of the two sequences can be accomplished by well-known methods, for example, using a mathematical algorithm. A preferred, non-limiting example of such a mathematical algorithm is described in Karlin et al., 90 PROC.NAT'LACAD. SCI. USA 5873-77 (1993), which is incorporated herein by reference. Such an algorithm is incorporated into the BLAST programs (version 2.2) as described by Schaffer et al., 29 NUCLEIC ACIDS RES. 2994-3005 (2001), which is incorporated herein by reference. When utilizing BLAST and Gapped BLAST programs, the default parameters of the respective programs can be used. In one embodiment, the database searched is a non-redundant database, and parameters for sequence comparison can be set at: no filters; Expect value of 10; Word Size of 3; the Matrix is BLOSUM62; and Gap Costs have an Existence of 11 and an Extension of 1.

In another embodiment, the percent identity between two amino acid sequences can be determined by using the GAP program in the GCG software package (available from Accelrys, Inc. of San Diego, Calif., at http://www.accelrys.com, as of Aug. 31, 2001) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be determined using a gap weight of 50 and a length weight of 3. Other preferred sequence comparison methods are described herein.

The invention also encompasses peptides having a lower degree of identity but having sufficient similarity so as to perform one or more of the same functions performed by a peptide encoded by a nucleic acid molecule of the invention (e.g., the ability to act as a substrate for a protein, e.g., a protein produced by a microorganism or by a mammal). Similarity is determined by conserved amino acid substitution. Such substitutions are those that substitute a given amino acid in a peptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al., SCIENCE 247:1306-10 (1990), which is incorporated herein by reference.

Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncations or a substitution, insertion, inversion, or deletion in a critical residue or critical region.

Amino acids in a peptide of the present invention that are essential for modification of a substrate can be identified by methods known in the art, such as site-directed mutagenesis or alanine-scanning mutagenesis (Cunningham et al., 244 SCIENCE 1081-85 (1989), which is incorporated herein by reference). The latter procedure introduces a single alanine mutation at each of the residues in the molecule (one mutation per molecule).

The invention also includes peptide fragments of the amino acid sequence of the various above-mentioned peptides or functional variants thereof. For example, fragments can be derived from a polypeptide comprising the sequence QKTTIKKLKH (H2) (SEQ ID NO: 9). Useful fragments include those that retain the ability to act as substrates for a protein (e.g., a protein produced by a microorganism).

Fragments can be discrete (not fused to other amino acids or peptides) or can be within a larger peptide. Further, several fragments can be comprised within a single larger peptide. In one embodiment, a fragment designed for expression in a host can have heterologous pre- and pro-peptide regions fused to the amino terminus of the peptide fragment and an additional region fused to the carboxyl terminus of the fragment.

The peptide of the substrate can be produced using standard recombinant protein techniques (See, e.g., Ausubel, F. M. et al. (“Current Protocols in Molecular Biology”, John Wiley & Sons, (1998) the entire teachings of which are incorporated herein by reference). In addition, the proteins of the present invention can also be generated using recombinant techniques. By testing with an ample supply of the protein to be detected and the substrate, the exact site of modification can be determined and a more specific substrate for that protein can be defined, if so desired. This substrate can also be used to assay for the presence of microorganisms of interest in order to assess a medical condition in a female mammal.

Peptides can be synthesized and conjugated with an enzyme (such as horseradish peroxidase (HRP), alkaline phosphatase (AP) or phenyl oxidase (PO)) or a simple dye molecule (e.g., blue dye #1) that allows for the determination of both the sensitivity and the specificity of a peptide for a particular microorganism. The choice of reporter dye dictates the speed of the diagnostic assay. For example, for conjugation to HRP, a 3-step process can be used: 1) labeling HRP with sulfosuccinimidyl-4-(N-maleimidomethyl) cyclohexane-1-carboxylate (SMCC) in sodium phosphate buffer, pH 7.5, to produce a maleimide form; 2) conjugating HRP maleimide to the peptide in phosphate buffer with 5 mM EDTA; and 3) coupling the HRP-peptide to microbeads. The coupling of the HRP peptide to the micro-beads can be performed with a crosslinker, EDC (1-ethyl-3-(3-dimethylaminopropyl) carbodiimide hydrochloride) in MES buffer. EDC conjugates the carboxyl groups on the bead to the amino terminus of the peptide. In the case of the slow reacting food grade dye, blue dye #1 can be synthesized with a maleimide to conjugate directly to the cysteine at the C-terminus of the peptide.

In some embodiments, substrate modification comprises cleaving at least a portion of the substrate, wherein the portion includes one of the colorimetric components and the cleaving results in a visible color change. For example, if the substrate includes both blue and yellow colorimetric components, the non-cleaved substrate can appear green. After a protein cleaves a portion of the substrate that includes the yellow colorimetric component, the cleaved portion is removed from the immediate presence of the uncleaved portion, leaving the non-cleaved portion to appear blue. The visible color change indicates the presence of the microorganism in the sample, while absence of a color change indicates the absence of the microorganism.

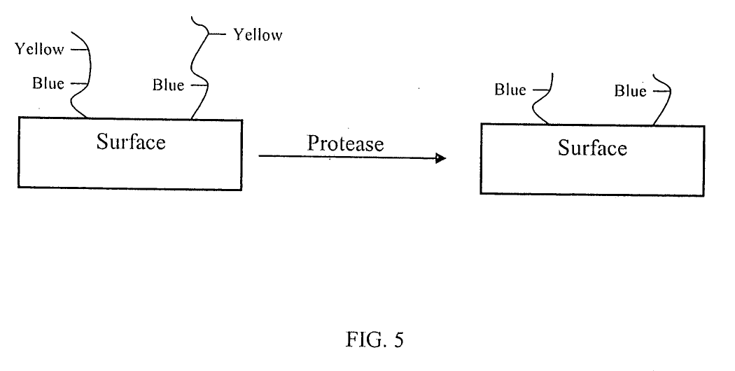

FIG. 5 illustrates a schematic of one embodiment of the invention. The unmodified substrates comprise a peptide, a yellow colorimetric component, and a blue calorimetric component. The unmodified substrate has a green hue. After modification by a protease, a portion of the peptide that includes the yellow calorimetric component is cleaved, leaving the substrate with only a blue colorimetric component. Hence, the modification of the substrate produces a signal in the form of a color change of green to blue.

In some embodiments, the modification of the substrate includes cleaving one peptide bond of the peptide. In other embodiments, the modification of the substrate includes cleaving at least one calorimetric compound from the peptide, resulting in a visible color change. In a further embodiment, the modification of the substrate includes hydrolyzing at least one peptide bond in the peptide and results in at least a portion of the peptide being cleaved from the substrate. The cleaved portion includes at least one of the colorimetric components, resulting in a visible color change.

The exact mechanisms employed to remove the cleaved portion of the substrate from the immediate presence of the non-cleaved portion can vary. For example, the cleaved portion can diffuse, absorb, adsorb and/or migrate into the surrounding environment (e.g., a pad) or it can be washed away with a liquid.

The device embodiments for these peptide-based sensors include a point-of-care device and a self-diagnostic device.

Colorimetric Components

The substrates can be labeled with at least one colorimetric component. As used herein, the term “colorimetric component” includes a visible dye, for example, a chromogenic, fluorescent or luminescent dye, or an enzyme, which is capable of attachment, for example, to a substrate, protein, or peptide.

In some embodiments, the substrates are labeled with at least two calorimetric components (e.g., at least 2, 3, 4, 5, 7, 10, 15, or more calorimetric components). The colorimetric components produce a signal (e.g., a visible change in color) if the substrate is modified (e.g., cleavage of the peptide and/or one or more calorimetric components from the substrate). In this way, the colorimetric components act as a label or tag to indicate the presence or absence of the modification. In some embodiments, the signal is a visible change in color. In other embodiments, the signal is a change in color that is detectable within the visible band of the light spectrum (e.g., from ˜700 nm to ˜400 nm). By attaching a larger number of colorimetric components to the substrate, a more visible color or color change can be produced. In further embodiments, the substrates are labeled with at least two dissimilar colorimetric components. Such embodiments allow for the possibility of producing two or more different changes of color or hue.

Dyes

Many types of dyes, for instance reactive dyes and fiber reactive dyes (referred to herein simply as “reactive dyes”) are available commercially (e.g., from dye manufacturers such as DyStar Textilfarben GmbH & Co. Deutschland KG, Frankfurt, Germany, and chemical companies, such as Sigma Aldrich, Acros, Molecular Probes, and ICN) and are suitable for use as calorimetric components. Reactive dyes can be, for example, colorimetric or fluorescent. The type or specific species of dye(s) selected for a detection method, application, or article of manufacture will depend on the properties of the dye (e.g., a molar extinction coefficient) and the environment in which it is to be used. The colorimetric component can be a dye having low toxicity (e.g., a food-grade dye).

Reactive dyes are colored compounds that contain one or two reactive groups capable of forming covalent bonds between, for example, the dye and a protein, peptide, substrate, colorimetric components, a solid support, or a collector. Approximately 80% of all reactive dyes are based on the azo chromophore. However, bacteria can sometimes decolorize azo dyes over time (e.g., 24 hours), so non-azo dyes are preferred. Fiber reactive dyes are colored compounds that have a reactive group capable of forming a covalent bond with a fiber. These dyes have been historically used in the textile industry. Examples of other suitable dyes include those that are approved for use in foods, drugs, cosmetics, or medical devices (e.g., contact lenses or sutures) by the U.S. Food and Drug Administration (e.g., Blue Dye #1 (Erioglaucine), Reactive Black 5, Reactive Blue 21, Reactive Orange 78, Reactive Yellow 15, Reactive Blue 19, Reactive Blue 4, Reactive Red 11, Reactive Yellow 86, Reactive Blue 163, and Reactive Red 180); mono- and di-halogentriazine dyes (e.g., mono- and di-fluorotriazine dyes; mono- and di-chlorotriazine dyes; mono-(m′-carboxypyridinium) triazines; Reactive Blue 4; Reactive Yellow 86; dyes in the PROCION® line of dyes, dyestuffs, and coloring matters, which are available from BASF; and the CIBACRON™ line of coal tar colors, which are available from Ciba-Geigy); 2,4,5 trihalogenopyriminidines; 2,3 dihaloquinoxalines; N-hydroxysulfosuccinimidyl (sulfo-NHS) ester functionalized dyes; N-hydroxysuccinimidyl (NHS) functionalized dyes; vinyl sulfone dyes (e.g., REMAZOL® line of coal tar dyestuffs, such as REMAZOL® Blue, produced by DyStar Textilfarben GmbH & Co. Deutschland KG; and Reactive Black 5); and sulfonyl chloride dyes (e.g., lissamine rhodamine, and dabsyl chloride); tetrafluorophenyl ester functionalized dyes; isothiocyanate functionalized dyes; and iodoacetyl functionalized dyes. The invention also encompasses dyes that are structurally equivalent to the dyes listed herein.

The structure of Erioglaucine (also known as FD&C Blue 1, Acid Blue 9, Brilliant Blue FCF, or N-ethyl-N-[4-[[4-[ethyl[(3-sulfophenyl)methyl]amino]phenyl](2-sulfophenyl)methylene]-2,5-cyclohexadien-1-ylidene]-3-sulfo-, inner salt, disodium salt, CAS number [3844-45-9]) is: 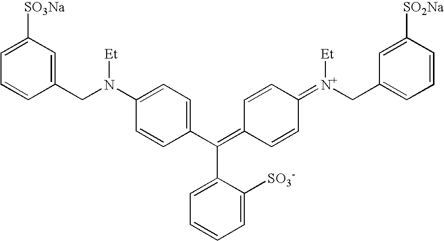
A sulfonyl chloride form of this dye may be prepared via methods known to those skilled in the art. The chemical structures of two possible isomers of the sulfonyl chloride of Erioglaucine are: 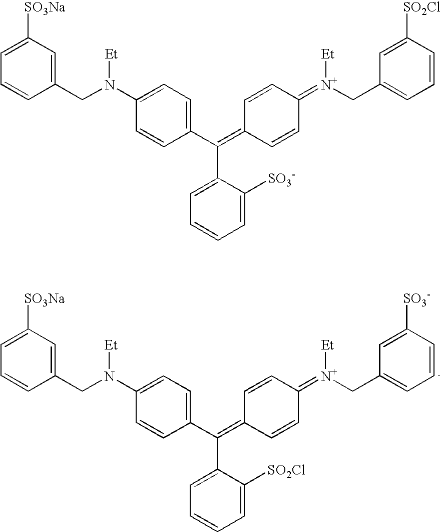
The names of these dyes are N-ethyl-N-[4-[[4-[ethyl[(3-(chlorosulfonyl)phenyl)methyl]amino]phenyl](2-sulfophenyl)methylene]-2,5-cyclohexadien-1-ylidene]-3-sulfo-, inner salt, sodium salt and N-ethyl-N-[4-[[4-[ethyl[(3 -sulfophenyl)methyl]amino]phenyl](2-(chlorosulfonyl)phenyl)methylene]-2,5-cyclohexadien-1-ylidene]-3-sulfo-, inner salt, sodium salt.

Sulfonyl chlorides are known to react preferentially with primary amine groups, such as those found on lysine groups or on the N-terminus of peptides and proteins. Thus, the dyes shown above may be used directly to label peptides. Alternatively, via methods known to those in the art, the sulfonyl chloride form of Erioglaucine may be further chemically modified to present other functional groups, such as NHS esters, iodosuccinimides, isothyocyanates, maleimide or other reactive groups. Examples of such chemical modifications of other dyes are given in U.S. Pat. No. 5,393,514, U.S. Pat. No. 5,846,737 and U.S. Pat. No. 5,798,276, the entire teachings of which are all incorporated herein by reference.

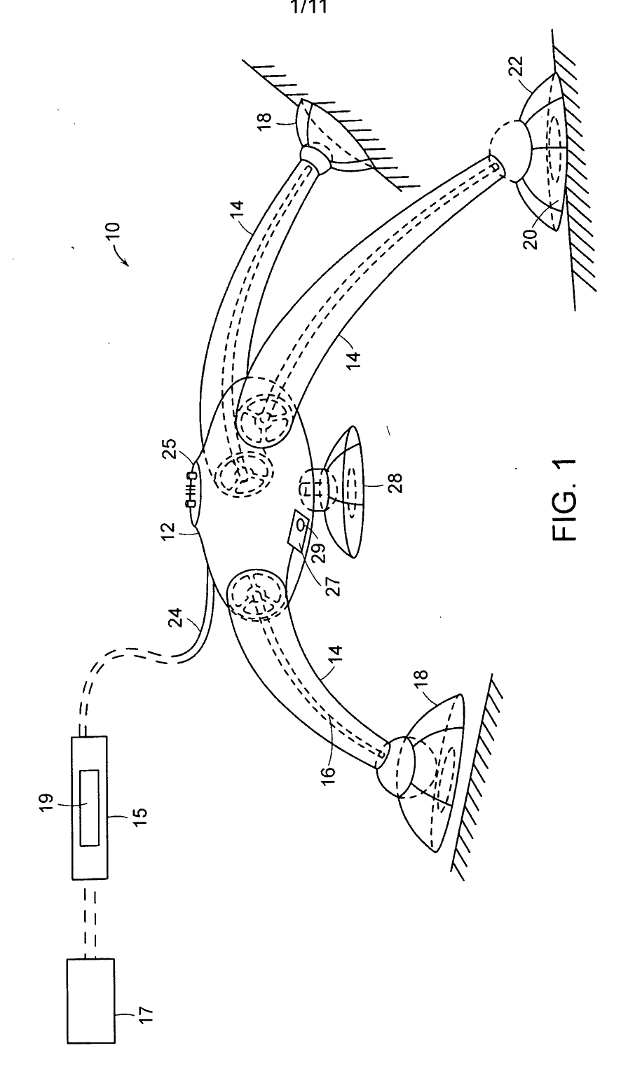

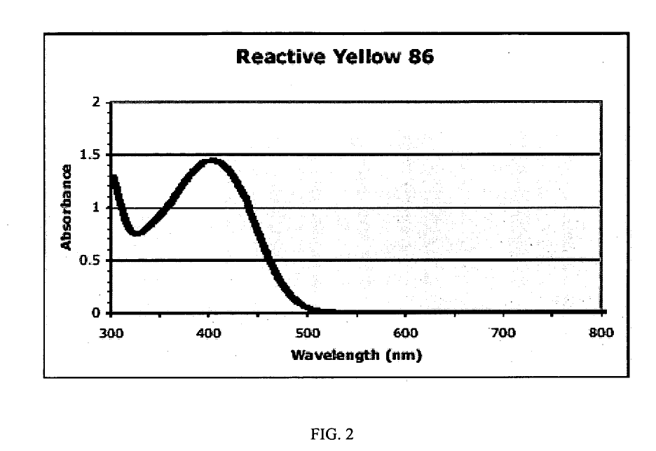

Fiber reactive dyes are based on chlorine or fluorine leaving group chemistries and are known as chloro- or fluoro-triazinyl dyes. Reactive dyes range from very low reactivity to highly reactive (such as CIBRACRON™ F and PROCION® MX) under a variety of temperature ranges. The reactive group is a triazinyl ring (a six-sided ring with three nitrogens). The reaction is considered a nucleophilic bimolecular substitution mechanism. It is a specific base-catalyzed addition of the nucleophilic functional group of the substrate to the electrophilic center of the reactive group of the dye. Reactive Blue 4 and Reactive Yellow 86 have the following structures: 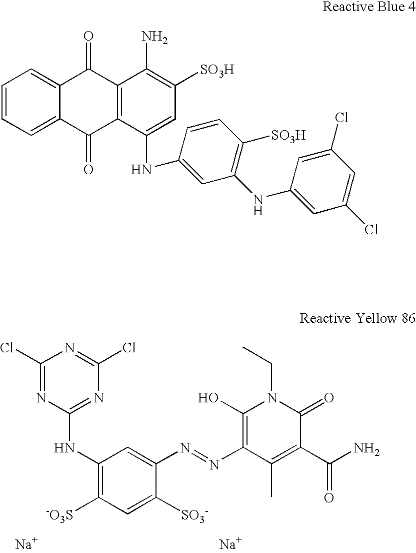
The UV/Visible spectra in water of triazine dye Reactive Blue 4 are illustrated in FIG. 1, while the spectra of triazine dye Reactive Yellow 86 are illustrated in FIG. 2.

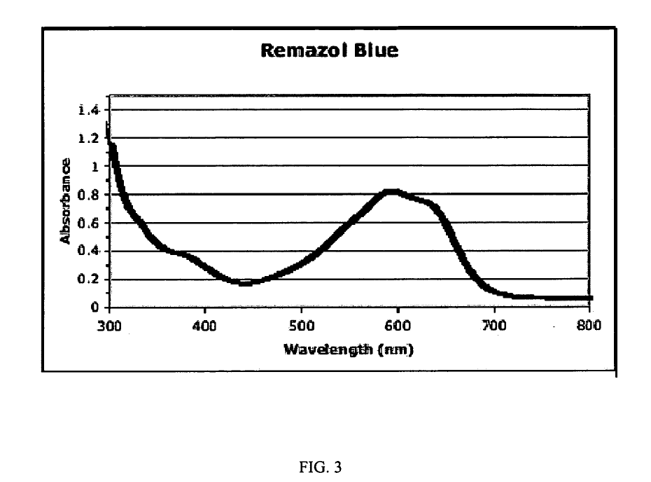

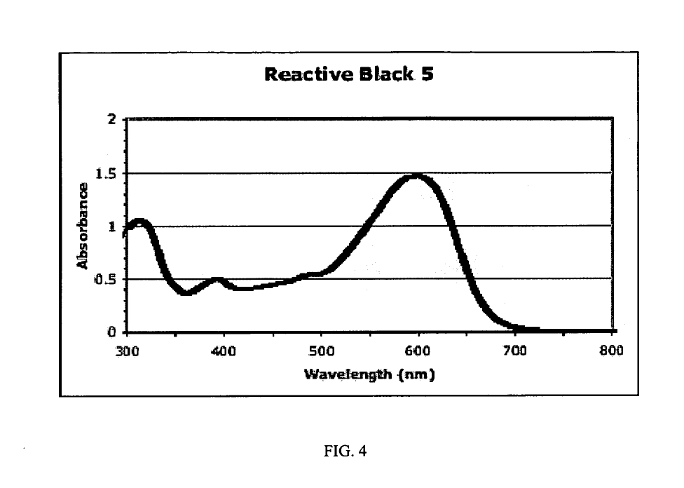

Vinyl sulfone dyes react via a nucleophilic addition mechanism, where there is frequently an elimination step before the addition step, resulting in the formation of a vinylic intermediate. Typically, there is a base-catalyzed elimination of a nucleofugic leaving group followed by a base-catalyzed addition of a nucleophilic functional group of the substrate. REMAZOL® dyes are examples of vinyl sulfone dyes utilizing the reactive group: —SO₂—CH₂—CH₂—OSO₃Na. Reactive Blue 19 and Reactive Black 5 have the following structures: 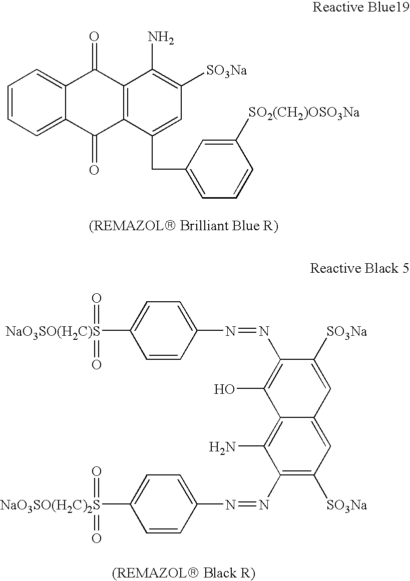
The UV/Visible spectra in water of REMAZOL® Brilliant Blue R are illustrated in FIG. 3, while the spectra of REMAZOL® Black B vinyl sulfone are illustrated in FIG. 4.

Sulfonyl chlorides are reactive sulfonic acid derivatives. Reaction of sulfonyl chloride compounds with a primary amine-containing molecule proceeds with the loss of chlorine and the formation of a sulfonamide linkage. The structure of the sulfonyl chloride dye, lissamine rhodamine B sulfonyl chloride (i.e., Xanthylium, 9-[4-(chlorosulfonyl)-2-sulfophenyl]-3,6-bis(diethylamino)-, inner salt, available from Molecular Probes, Inc., Eugene, Oreg., CAS Number/Name: 62796-29-6), is:

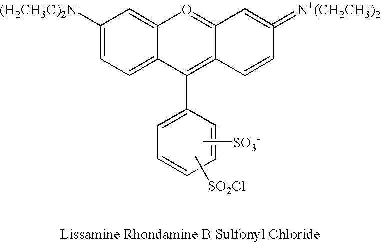

N-hydroxysulfosuccinimidyl (sulfo-NHS) ester functionalized dyes are water-soluble and react with primary amine-containing molecules to form an amide bond with the loss of the sulfo-NHS group. N-hydroxysuccinimidyl (NHS) functionalized dyes are also reactive to amine groups. The structure of the dye functionalized with NHS ester, BODIPY® FL, SSE (i.e., 4,4-difluoro-5,7-dimethyl-4-bora-3a,4a-diaza-s-indacene-3-propionic acid, sulfosuccinimidyl ester, sodium salt; available from Molecular Probes, Eugene, Oreg.), is:

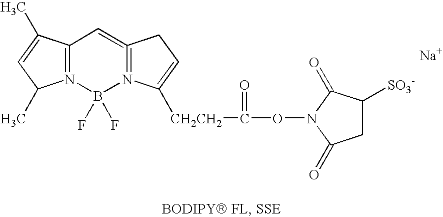

The colorimetric component(s) is attached to the substrate by methods known in the art, such as those described by Greg Hermanson in BIOCONJUGATE TECHNIQUES (1996), available from Academic Press, San Diego, Calif., the teachings of which are incorporated herein in their entirety by reference. In some embodiments, the colorimetric components are covalently attached to the peptide. For example, a protective group can be used to block one of the attachment sites on a peptide chain while a first colorimetric component is attached (e.g., a triazine dye attached to a serine group or a vinyl sulfone dye attached to a cysteine group). After the first colorimetric component is attached, the protecting group is removed in order to attach a second colorimetric component. Surfactants, such as TRITON®-X100, can be used to promote the attachment of the colorimetric components to the peptide chains and/or improve solubility.

The extent to which the substrate is labeled with colorimetric components will vary and can depend on many factors such as, for example, the needs of the application in which the substrate is to be used, manufacturing requirements, and the desires of the practitioner of this invention. One method of characterizing the level, or amount, of labeling a substrate includes, is to refer to its “dye-to-substrate ratio.” As used herein, the term “dye-to-substrate” ratio is the molar ratio of the molar concentration of dye to the molar concentration of substrate and is a means of characterizing the level of labeling of the substrate as well as the efficiency of the labeling reaction. For example, a dye-to-substrate ratio of 1.0 would signify that, on average, each substrate is labeled with one calorimetric component. A low dye-to-substrate ratio can signify an incomplete labeling of the substrate (i.e., many peptides have no dye attached to them). In some embodiments, the range of the dye-to-substrate ratio depends on the number of amino acids on the peptide that are to be labeled with the color component. For example, if two sites are to be labeled, then a complete labeling reaction would render a dye-to-substrate ratio of about 2. Suitable dye-to-substrate ratios depend on the exact application. For example, the dye-to-substrate ratio can affect the clarity of the signal, attachment of the substrate to a solid support, and other aspects to, for example, a biosensor, that can be varied depending on the needs of the practitioner of the invention.

A suitable dye-to-substrate ratio can be determined through experimentation. For example, the peptide of the substrate can be synthesized to add or subtract extra amino acid groups that are suitable targets for attaching the colorimetric components. In this manner, the dye-to-substrate ratio can be varied, and the resulting substrates can be tested to determine what ratio produces acceptable results for a given application. For example, if a peptide of the substrate contains two cysteine groups at one end that are good targets for vinyl sulfone dyes, this allows for the attachment of two colorimetric components on each peptide. By adding a second colorimetric component, the resulting signal produced by the substrate will be brighter (i.e., having a larger color intensity and/or sharper contrast) than if only one colorimetric component was attached to the substrate. This brighter color may be more favorable for a given application. For example, a dark blue dye may easily be seen on a solid support, but a yellow dye may require more dyes per peptide to create the desired level of color. However, too many dyes on one end of the peptide may create a steric hindrance if, or when, the peptide is attached to a solid support. There can be an optimum dye-to-substrate ratio or ratio range for each substrate and/or application. Acceptable results include fast hydrolyzation of the substrate and/or enhanced solubility.

In one embodiment, in order to make sensor diagnostics with robust colors, a PAPA peptide can be synthesized on P4 paper and conjugated to rhodamine dye.

Enzymes

In some embodiments, enzymes are colorimetric components. As used herein, a reporter enzyme is any enzyme conjugated with a peptide which, upon hydrolysis, leads to the activation of a catalytic process leading to a detectable signal (e.g., a visible color change). Such enzymes include, but are not limited to those described herein, such as horseradish peroxidase (HRP), phenol oxidases (e.g., laccase, CotA), alkaline phosphatase (AP) and galactosidase (see also, for example, published international application PCT/US2004/028675 (WO2005/021780); Title: “Signal Amplification Using A Synthetic Zymogen,” which is incorporated herein by reference in its entirety).

Solid Supports

In some embodiments, the substrate is attached or coupled to (e.g., applied to, connected, incorporated in, brought to close proximity with) a solid support. The solid support can have at least one absorbent layer of material that absorbs bodily fluids, and at least one non-absorbent layer to prevent leakage of the bodily fluids. In one embodiment, the bodily fluid can be urinary fluid. In another embodiment, potential (bodily) samples on/in which the presence or absence of proteins can be detected include a body fluid (e.g., vaginal fluid, urine; a piece of hair; a piece of tissue (e.g., a medical tissue sample). Examples of suitable solid supports include a bead, a feminine napkin, any material that needs to be sterile or free of microbial contamination, an article that contains or collects the sample (e.g., a urine collection bag, a blood collection bag, a plasma collection bag, a test tube, a body fluid collection tube, a test tube, a catheter, a swab, a swab carrier, a dipstick, or a well of a microplate), a polymer, a membrane, a resin, glass, a sponge, a rigid probe or capillary, a point of care ruler, a disk, a scope, a filter, a lens, foam, cloth, paper, a wipe, a suture, a speculum and a bag. Other examples of suitable solid supports include feminine hygiene products and consumer products including a diaper, a pad, a feminine napkin, and/or a tampon.

In some embodiments of the invention, the solid support is made from materials suitable for sterilization. Examples of suitable methods of sterilizing the solid support include gamma irradiation treatments, ethylene oxide treatments, and autoclaving. In some embodiments, the solid support includes a calorimetric component and/or the solid support is made of a colored material.

In preferred embodiments, the solid support is a medical product that contacts a patient or body tissue or fluid samples from a medical patient. For example, in some embodiments, the solid support is a medical device or product (e.g., a speculum) having a sample port that allows access to a fluid sample (e.g., body fluid, such as urine or vaginal fluid), a wicking agent to draw the fluid into the device or product, an assay chamber in which the detection takes place, and a viewing port that allows a practitioner to see the signal that indicates the presence of a microorganism. Such solid supports provide a diagnostic or point-of-care (POC) device that can be used by health care providers to quantify and qualify the presence of any infectious or pathogenic microorganisms within a fluid. For example, such embodiments could be used to determine if a fluid has a critical infection level, e.g., 10⁵ CFU per ml pathogens (or greater).

For example, in a lab specimen collection container, a blue color of a sensor can indicate the presence of one or more specific types of microorganisms in the lab specimen container. Similarly, a sensor could be placed in a swab sample container to indicate the presence of microorganisms in a sample that has been placed into the container.

In some embodiments, sensors containing specific markers for pathogens are bound to a collar that is placed at the back of a swab (e.g., a cotton, polyurethane, or polyester swab), a medical device (e.g., a speculum) or sampling device. As used herein, a sampling device is any device used to acquire a sample from a person (e.g., a patient), such as a fluid or tissue sample. One example of such a device is a capillary tube. The swab draws the fluid of a sample up along the outside of the stem. The fluid then passes from the top of the swab to the collar sensor, and if the microbial proteins of interest are present in the fluid, a signal is produced by the sensor (e.g., a color change), thereby allowing a practitioner to assess a medical condition in a female mammal. In the case of a simple color dye labeled peptide, the free diffusing color is collected into a membrane that preferentially binds the dye leaving group with a strong affinity. In the case of a zymogen-peptide conjugate, the hydrolysis of the peptide leads to either the activation of the zymogen or the movement of the zymogen into an area that is clearly visible, thereby producing an amplification of the color signal.

In one example, a control contact swab was not exposed to a fluid sample, while the test contact swab was exposed. The sensor attached to the test swab changed color, indicating the presence of the microorganism of interest in the sample and providing for an assessment of a medical condition in a female.

In another embodiment, sensors are included on a cleansing wipe, a sponge, a tampon, a napkin, a liner, or a pad that could be used, for example, to clean, or be applied to, the pubic area of a female mammal to detect the presence of microorganisms and provide an assessment of a medical condition in the female. The cleansing wipe, sponge, napkin, tampon, or pad can comprise an absorbent material such as cloth, paper, cotton, sponge, or non-woven fibrous material. In one embodiment, a sensor would be attached or incorporated on the wipe or sponge in a pattern that would provide uniform coverage for detecting the proteins from an infected tissue or fluid. In some embodiments, the sensors are printed on the material in a pattern, such as waves, mesh, a grid, or spots. Preferably, the signal produced by the substrate is easily identifiable from the typical colors of the sample (e.g., vaginal or urinary) fluid. In further embodiments, the cleansing wipe, a sponge, a tampon, napkin, liner or a pad includes a collector for collecting the colorimetric component in order to produce a visible signal. In some embodiments, the wipe, sponge, tampon, napkin, liner or pad is also used to spread a therapeutic or antibiotic substance.

In yet another embodiment, the sensors are placed at the tip of a cylindrical rigid probe or capillary used to sample vaginal or urinary fluids. The probe is rigid enough to be inserted into the vagina and draw up fluid into a hollow chamber that contains the sensors. In some embodiments, the probe includes a broad spectrum of sensors, a series of specific sensors, or both. In some embodiments, the chambers are made of soft or hard clear materials (such as glass, silicone, or other materials with similar properties). The inner chamber of the probe draws up the liquid by capillary action and also contains membrane filters that include colorimetric sensors that are specific to each microorganism or pathogen of interest. Optionally, the probe or capillary includes an absorbent wick comprising polyurethane or other suitable material that is used to draw the sample fluid into the membrane sensor regions. A rigid capillary probe can include a plurality of sensors that display different color changes.

In another embodiment, the sensors are incorporated into a fine strip, thin film, mesh, tape, speculum or suture material that is placed on or near female genitalia to collect fluid. In some embodiments, the sensors produce a signal within minutes of detecting the presence of a microorganism(s) of interest. In some embodiments, the strip, film, mesh, speculum or suture is made of a non-absorbent material (e.g., nylon or polyethylene fibers). In other embodiments, it is made from an absorbent material (e.g., polyurethane). In use, the thin films, mesh, sutures or other material can be placed in a plastic or glass test tube carrier commonly used to transport swab samples to a laboratory, thereby providing a sensor system for the caregiver and a vessel for a confirmatory readout within a hospital laboratory.

In some embodiments, the point of care (POC) sensor device can be incorporated into a sampling device, specimen bag, jar or collection tube that can be used to transfer a sample (e.g., a swab or fluid sample) to a lab. In further embodiments, the peptides are implanted onto a thin film and/or directly impregnated onto the sample jar or bag. Optionally, non-ionic detergents (e.g., HECAMEG®, TRITON®-X100, or TWEEN®) and/or other reagent(s) (e.g., buffers, such as PIPES (pKa=6.76), DNAse I, and/or non-porous glass or metal beads) are included in the POC sensor and/or specimen bag or jar in order to control the optimal activity of the sensors, to hydrolyze the DNA from the tissue sample and prevent it from becoming too viscous, to macerate the tissue by gentle swirling, to permeablize the tissue sample to promote detection of any microorganism of interest, and/or to improve the homogeneity of the biopsy/tissue sample.

In some embodiments, the POC device is a disposable item that would be used once and then placed in biohazard waste. Upon autoclaving, the device would be destroyed and the patient's information would be kept confidential.

In some embodiments, the substrate is adhered, attached, coupled, or bound to the solid support. Methods for doing so are known in the art. For example, the substrate can be attached to the solid support through noncovalent interactions (e.g., hydrophobic interactions, hydrophilic interactions, electrostatic interactions, or through a sorption process) or by covalent binding. In further embodiments, the substrate includes hydrophobic leaving groups and is non-covalently bound to a hydrophobic surface of a solid support. In other embodiments, hydrophilic or hydrophobic substrates are coupled to surfaces by disulfide or primary amines, thiol groups, carboxyl groups, hydroxyl groups, or with the use of crosslinkers (e.g., sulfo EMCS (N-Maleimidocaproyloxy sulfoxuccinimide ester), available from Pierce Biotechnology, Inc., Rockford, Ill.). In yet another embodiment, the substrate is coupled to a solid support using non-essential reactive termini such as free amines, carboxylic acids, or thiol groups that do not effect the substrate's interaction with a protein produced by a microorganism. Free amines can be coupled to carboxyl groups on the substrate using, for example, a 10-fold molar excess of either N-ethyl-N′-(3-dimethylaminopropyl) carbodiimide hydrochloride or N-cyclohexyl-N′-2-(4′-methyl-morpholinium) ethyl carbodiimide-p-toluene sulphonate for ˜2 hrs at ˜4° C. in distilled water, adjusted to a pH ˜4.5, to stimulate the condensation reaction to form a peptide linkage. Thiol groups can be reduced with Dithiothreitol or Tris (2-Carboxyethyl) Phosphine and then coupled to a free amino group on a surface with N-e-Maleimidocaproic acid (see D. G. Griffith et al., “N-Polymethylenecarboxymaleimides: A new class of probes for membrane sulfhydryl groups,” 134 FEBS LETT. 261-63 (1981), which is incorporated herein by reference). In another embodiment, the substrate is attached to the solid support by attractive interactions between the amino acids of the peptide and the solid support. For example, a peptide with consecutive histidine residues can be bound to a resin (e.g., SEPHAROSE®) containing nickel-NTA (e.g., they can be attached to a nickel-NTA resin). In yet another example, the solid support has a polar charge (e.g., a negatively charged membrane) and at least some portion of the substrate (e.g., one or more peptides of the substrate's peptide chain) has a polar charge opposite of that on the solid support. These opposite charges provide for the attachment of the substrate to the solid support.

In some embodiments, the solid support, if desired, can provide a plurality of derivatized binding sites for coupling to the substrate, for example, succimidyl ester labeled primary amine sites on derivatized plates (XENOBIND™ binding plates, available from Xenopore Corp., Hawthorne, N.J.). Optionally, coupling, for example, bovine serum albumin, thereto blocks unoccupied reactive sites on the solid support.

In some embodiments, the peptide can be attached or coupled to the support at any point or points on the peptide. In one embodiment, the peptide is covalently tethered to polystyrene, acrylate or agarose beads via the N terminus. A reporter enzyme or visible dye can be specifically coupled to the C-terminus end of the peptide.

Collectors

In some embodiments, a collector is used to remove the cleaved portions so that the signal is detectable. As used herein, a “collector” is a solid or surface comprising a property that attracts the cleaved portion of a substrate. In these embodiments, one or more of the cleaved portions can have a higher attraction or affinity for the collector than for the other portion of the substrate and/or the solid support so that the cleaved portions migrate, absorb, adsorb, and/or diffuse from the substrate and towards the collector or some point remote from the noncleaved portion of the substrate or the modified substrate. In some embodiments, the distance that the cleaved portion migrates is sufficient so that a detectable signal results. In some embodiments, the migration of the cleaved portion(s) results in a detectable signal.

In some embodiments, the collector for dyes can include a quaternary amine or DEAE charged membrane, support or resin, including Biodyne B (Pall Life Sciences), SB6407 (Pall Life Sciences), and positively-charged PVDF (Millipore). In some embodiments, those membranes collect the food grade dye Blue dye #1. Since PVDF is transparent when wet, it is possible to see the collection of the color on the bottom surface. Some dyes prefer cationic and some dyes prefer anionic membranes. For example, some dyes such as Blue due #1 bind positively charged membranes such as DEAE or quaternary amines. In contrast, some dyes, such as Remazol brilliant blue, bind negatively charged membranes, such as ICE (Pall Life Sciences).

In some embodiments, an ionic membrane resin or support can be used to collect colors preferentially.

In some embodiments, the cleaved portion of the substrate can be captured on a collector. In further embodiments, the cleaved portion is captured on a colored collector. In still further embodiments, modification of the substrate results in a cleaved portion being captured on the surface of a colored collector, thereby producing or indicating a change in color of the solid support. That is, once the cleaved portion of the substrate is released, it is attracted to the collector by one or more forces (e.g., a force caused by an electrostatic or magnetic charge, hydrophobic interactions, or a chemical binding). In another embodiment, the collector causes the cleaved portion to migrate a sufficient distance from the substrate so that the color of the solid support is detectable.

As a non-limiting example, a substrate can include a first calorimetric component (e.g., a yellow colorimetric component attached to a peptide). Modification can include cleaving the substrate such that a first cleaved peptide portion includes the first colorimetric component and a second cleaved peptide portion does not include a colorimetric component. The first cleaved peptide portion can be attracted towards the collector, resulting in a visible color change of the collector (e.g., the collector can appear more yellow). If the substrate was originally attached to a solid support, the combination of the solid support and non-cleaved portion of the substrate can exhibit a change in color (e.g., appearing less yellow or becoming colorless).

In another non-limiting example, the first and second cleaved portion can be present in a liquid, and the migration of the first portion that includes the colorimetric component (e.g., yellow) towards a collector can result in the liquid exhibiting a change in color (e.g., appearing less yellow or becoming colorless).

In yet another non-limiting example, the unmodified substrate can include at least two colorimetric components (e.g., a yellow and a blue calorimetric component) and the modification can result in the first cleaved portion including the yellow colorimetric component and the second portion including the blue colorimetric component. Prior to substantial migration of the first calorimetric component, the close proximity of the first and second colorimetric components can provide a green appearance. As the first portion migrates towards a collector, the individual colors become more apparent. For example, if the second portion is attached to a solid support, the combination of the solid support and the second portion can change color (e.g., becoming more blue in hue) as the first portion migrates towards a collector and away from the solid support and the second portion. If, for example, the original substrate was included in a liquid, the liquid will continue to appear green immediately after the modification. However, as the first cleaved portion migrates towards the collector, the remaining combination of fluid and second cleaved portion can change color (e.g., becoming more blue in hue). Optionally or additionally, the modification of the substrate can result in the collector producing a color change (e.g., the collector exhibiting a more yellow color as the yellow first cleaved portion collects onto the surface of the collector).

In some embodiments, the modification results in a visible color change in a predetermined pattern. For example, the modification can result in the appearance, disappearance, and/or color change of a shape (e.g., a symbol, letter, number, bar code, code or word). This can be accomplished with, for example, attaching substrates onto a solid support in a pattern (e.g., a star, cross, or plus sign). For example, the solid support material can be blue and the substrates with yellow calorimetric components can be arranged on the solid support in the shape of a star. Prior to modification, the solid support appears to have a blue background with a green star (with the green hue of the star arising from the mixture of the yellow hue of the substrate and the blue hue of the solid support). Modification results in the cleavage of the yellow calorimetric components from the substrate and is indicated by the fading of the green star, leaving the solid support to appear blue. In another embodiment, a collector is employed and the collect is formed in such a way that cleaved portions will be attracted to a predetermined area of the collector. For example, the substrates can include a yellow colorimetric component and the collector can comprise a blue material. Modification of the substrate results in the yellow colorimetric components collecting on the collector in a predetermined shape, such as the shape of a cross. In this manner, modification of the substrates is indicated by the appearance of a cross with a green appearance (due to the combined blue hue of the collector and the yellow of the colorimetric components). In yet another embodiment, modification of the substrate results in the appearance, disappearance, and/or visible change of color in one or more predetermined shape on both a solid support and a collector. Optionally, many different types of substrates, collector surfaces, and solid support surfaces are used so that a plurality of modifications and/or microorganisms can be detected. This invention encompasses the use of more than one shape, combination of shapes, and color shifts on one or more collectors and/or solid supports which will be apparent to one skilled in the art.

The examples described herein are not meant to be limiting in any way, and other combinations of solid support(s), type, number, color or hue of calorimetric components, and migration-inducing attractive and/or repulsive collectors are also encompassed by this invention.

In one embodiment, the invention includes a method of detecting the modification of a substrate, wherein an unmodified substrate comprises a peptide with at least one calorimetric component, wherein the method comprises the steps of a) exposing the substrate to a sample under conditions that will result in the modification of the substrate, wherein the modification of the substrate comprises cleaving at least a portion of the peptide that includes at least one colorimetric component and the cleaved portion migrates toward a collector, wherein the migration results in a visible color change; and b) detecting the presence or absence of the visible color change, wherein a visible color change indicates the modification of the substrate and the absence of a visible color change indicates an absence of the modification of the substrate.

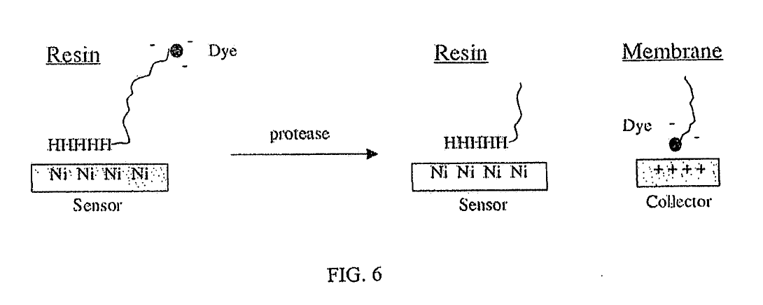

FIG. 6 illustrates a schematic of a metal chelation embodiment of the invention. A colorimetric component, in this case a dye, is included on a peptide that is attached to a nickel-NTA resin solid support. A protease cleaves a portion of the peptide, and the cleaved portion has a greater affinity for a membrane collector than for the original surface. As the dye migrates towards the collector, the remaining peptide and solid support produce a visible color change. Optionally, the collector produces a signal as the dye migrates toward or onto the collector. See, for example, U.S. Provisional Application Ser. No.: 60/771,107, “Ultra-Sensitive Biosensors and Methods of Use Thereof,”, Sanders, M. et al.

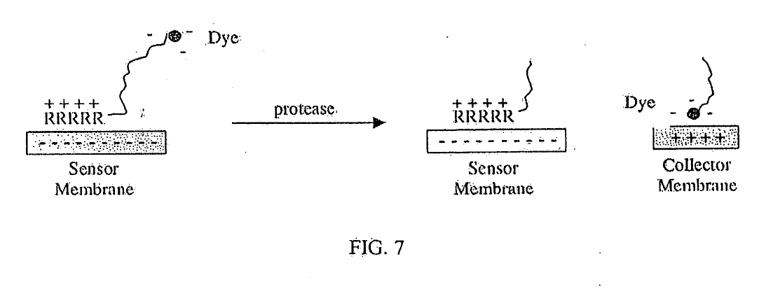

FIG. 7 illustrates an embodiment of the invention where a charged membrane is coupled to a substrate that includes a colorimetric component. A protease cleaves a portion of the peptide, and the cleaved portion has a greater affinity for the membrane collector than for the original surface. As the dye migrates towards the collector, the remaining substrate and solid support produce a visible color change. Optionally, the collector produces a signal as the dye migrates toward it.

The collectors can be made of any suitable material that facilitates the migration of cleaved portions of the substrate. For example, the collector can include a membrane, a resin, a polymer, a film, glass, or a chelating material. In some embodiments, the collector is attached to a solid surface, such as a tampon, a feminine pad, a feminine napkin, a diaper, a speculum, a wipe, any material that needs to be sterile or free of microbial contamination, an article that contains or collects the sample (e.g., an article inserted in a female mammal's body, a urine collection bag, a blood collection bag, a plasma collection bag, a test tube, a body fluid collection tube, a disk, a scope, a speculum, a filter, a sampling device, a test tube, a catheter, a swab, a swab carrier, a polymetric article, a dipstick, or a well of a microplate), a well of a microplate, a polymer, a membrane, a resin, glass, a sponge, a disk, a scope, a filter, a lens, foam, cloth, paper, a suture, a bead, a film, a chelating material, a layer of an absorbent pad, tampon or diaper, made from materials suitable for sterilization. Examples of suitable methods of sterilizing the collector include gamma irradiation treatments. In some embodiments, the collector includes a colorimetric component or is made of a colored material.

Sensors

In other embodiments, the invention includes a sensor (e.g., a biosensor) for detecting the presence or absence of proteins, enzymes, or microorganisms, thereby allowing for an assessment of a medical condition in a female mammal. These sensors can incorporate methods of the invention as described herein. In one example, the sensor comprises a solid support and at least one detectably labeled substrate bound to the solid support. In one embodiment, the substrate is covalently bound to the solid support. In another embodiment, the substrate is adhered or attached to the solid support via hydrophobic, hydrophilic, and/or electrostatic interactions between the solid support and the substrate. In yet other embodiments, the substrate is adhered or attached to the solid support through adsorption or absorption. In other embodiments, the substrate includes a peptide and at least two colorimetric components attached to the peptide. The peptide specifically interacts or reacts with the protein of interest. In some embodiments, the colorimetric components are covalently attached to the peptide. In some embodiments, the sensor can be EXPRESSDETECT® sensors.

In some embodiments, the sensor includes at least one substrate that specifically interacts or reacts with the protein, enzyme, or microorganism and at least two calorimetric components covalently attached to the peptide. The interaction or reaction results in the substrate producing a detectable signal to indicate the presence of the protein. In further embodiments, the sensor detects one or more (e.g., at least ˜2, at least ˜5, at least ˜10, at least ˜20, at least ˜30, at least ˜50, at least ˜75, or at least ˜100) proteins described herein and produces a signal (e.g., a visible color change) to indicate the presence of the proteins.

In some embodiments, this invention includes a sensor for detecting the presence of at least two proteins, including a first protein and a second protein. For example, the sensor of the present invention can include one or more substrates (for example, at least ˜2, at least ˜5, at least ˜10, at least ˜20, at least ˜30, at least ˜50, at least ˜75, or at least ˜100 substrates) that can interact with one or more produced and/or secreted proteins. In one example, the sensor comprises a solid support, at least one detectably labeled first substrate, and at least one detectably labeled second substrate. The detectably labeled substrates are attached to the solid support. The first substrate includes a first peptide and at least two colorimetric components attached to the first peptide. The first substrate specifically reacts with the first protein. Similarly, the second substrate includes a second peptide and at least two calorimetric components, and the second substrate specifically reacts with the second protein.

In some embodiments, at least three of the four colorimetric components attached to the first and second substrates are dissimilar. In these multi-colored embodiments, the sensor can undergo two or more distinct visible color changes to indicate the presence of one or more distinct proteins and/or microorganisms. For example, if one type of substrate is designed to react with one type of protein while a second type of substrate is designed to react with a different protein, and both types of substrate are included on the solid support, a plurality of color changes can be designed to indicate the presence of a plurality of different proteins and/or species of microorganisms.

One method of making the sensor of the present invention is to first determine a specific substrate that can interact with a specific protein characteristic of the microorganism to be detected. The determined specific substrate is labeled with one or more calorimetric components and attached to a solid support. Should the substrate come into contact with the specific protein secreted or expressed by the microorganism of interest, the protein modifies the substrate in a manner that results in the detection of such a modification. For example, as described herein, the modification can produce a visible change in color.

Preferably, the portion of the sensor that comes into contact with the sample will not adhere excessively to the sample, so as to allow for the easy removal of the sensor from the sample. For example, if the sensor comprises a speculum or a feminine napkin, such as a pad, the sample contacts the speculum, feminine napkin or pad for a time sufficient for the protein to react with the substrate.

The present invention can be used to detect the presence or absence of any enzyme, e.g., pathogen-specific enzyme, described herein. For example, the methods and/or sensors can be used to detect the presence or absence of lipase enzymes secreted by pathogenic bacteria. It has been discovered that certain bacteria secrete lipases into their environment as part of their survival and/or virulence mechanisms. The lipases serve to break down lipids in the growth environment in order to release nutrients. Lipases may also play a role in disarming mammalian host defenses during infection. Synthetic substrates for these secreted enzymes can be employed to detect the presence of those pathogenic bacteria that secrete them. By using a substrate comprising at least one synthesized lipid and two or more calorimetric components, it is possible to create substrates that will change color as they are hydrolyzed by secreted lipases. This color change reaction forms the basis of a microbial sensor, which can be incorporated into such items as consumer products described herein (e.g., a feminine napkin).

In another example, the invention can be used to detect the presence or absence of a microorganism by detecting the presence or absence of autolytic enzymes associated or produced by a microorganism. Autolysins are enzymes that degrade peptidoglycan, a component of the bacterial cell envelope. Autolytic enzymes serve to break down peptidoglycan, be it that of the parent organism, as part of cell division and turnover functions, or as a means to breakdown cell walls of competing bacteria. When labeled with two or more colorimetric components, a substrate that comprises synthetic peptidoglycan subunits (such as, but not limited to, N-acetyl-β-d-glucosaminide) serves as an indicator that can form the basis of a sensor.

In another example, the methods and/or sensors of the present invention can be used to detect the presence or absence of beta galactosidase on the surface of the cell of a microorganism (e.g., bacteria). Most bacterial species express beta galactosidase as a cytoplasmic enzyme involved in the metabolism of lactose as an energy source. Certain species of Streptococcus, however, display the enzyme on the surface of the cell. A substrate comprising a molecule that acts as a substrate for beta galactosidase and at least two colorimetric components, (including, but not limited to, ortho nitrophenyl β-D-galactopyranoside) could thus be used as a means of detecting microorganisms (e.g., streptococci) in the environment.

In one embodiment, the sensor is for use in a healthcare or home-use setting and is suitable for detecting microorganisms and proteins to assess a medical condition in a female mammal.

In some embodiments, this invention features kits for detecting proteins, enzymes, and/or microorganisms. These kits incorporate the methods and sensors of this invention. In one example, a kit includes a sensor for detecting the presence or absence of a protein in a sample, and at least one reagent for detecting the substance. In some embodiments, the kits include a collector.

One example of a method for developing an assay for detecting a microorganism that produces at least one protein that is secreted or presented on the surface of the microorganism and a method for using the assay to detect pathogenic microorganisms producing the protein(s) now follows. This method is not meant to be limiting in any way, as other methods are known in the art.
Step 1) Define an amino acid sequence (or sequences) that uniquely identifies the microorganism of interest. Alternatively, an (one or more) amino acid sequence that is unique to a specific group of pathogens, for example, fluid-specific pathogens, can be determined.

Select an amino acid sequence, for example, a protein, peptide, or polypeptide (marker sequence) that uniquely characterizes or marks the presence of the microorganism or group of microorganisms (for example, vaginitis pathogens) of interest. The selection can be performed utilizing a bioinformatic approach, for example, as described in detail below. One or more amino acid sequences that are unique to a specific microorganism can be determined.
Step 2) Obtain sufficient protein to determine conditions facilitating optimal modification of a substrate by the enzyme.

Isolate the protein (e.g., from the extracellular medium in which the microorganism to be assayed is growing, or from the cell membrane of the microorganism) using standard protein purification techniques, described, for example, in Ausubel (supra).

Alternatively, if the genetic sequence encoding the protein or the location of the genetic sequence encoding the protein are unknown, isolate and clone the genetic sequence encoding the marker amino acid of Step 1, or, first determine the genetic sequence, and then proceed as before.
Step 3) Determine the conditions for growth of the microorganism and for the production of a protein presented on the surface of the cell or secreted by the cell.

Determine medium required for growth of the specific microorganism of interest and for expression of its unique active protein into the medium. Also determine whether a second molecule, for example an enzyme, is required to convert the specific protein from an inactive precursor form to an active form.

Optionally, the protein(s) produced by the microorganism(s) in the growth medium are compared with samples taken from clinical samples to ensure that the microorganisms produce the protein in the environment(s) in which they are to be detected. For example, the proteins found in a sample taken from a hospital patient can be analyzed and compared with the proteins produced by the microorganism grown in the medium. In this manner, it can be confirmed that the microorganism produces the protein in an actual testing sample or environment and that the protein can form the basis for the detection method.
Step 4) Identify any specific substrate(s) of the active protein. Examples of potential substrates include proteins, peptides, polypeptides, lipids, and peptidoglycan subunits. Label each substrate with a detectable label, for example, a colorimetric component described herein, or any other detectable label known in the art.
Step 5) Increase the specificity of the protein-substrate interaction (optional) by determining the active or binding site of the protein (for example, using the colorimetric components as described above), then determining the genetic sequence useful for producing the active or binding site, and cloning the determined genetic sequence to generate a more specific substrate.
Step 6) Provide a sensor comprising one or more of the detectably labeled substrates identified herein for detection of the protein (e.g., protease) of the microorganism of interest.

As described above, the substrate can be attached to a solid support, for example, a feminine napkin or pad, or an article that holds the protein and substrate, for example, a body fluid collection tube or bag, a microplate well, a test tube, or any other solid support described herein.

Allow the protein(s) to come into contact with the substrate(s), and monitor the reaction for a modification of the substrate, as described herein. Modification of the substrate indicates that the protein produced and/or secreted by the microorganism is present in the reaction. In addition, the absence of modification of the substrate indicates that the protein is not present in the sample. If the microorganism or protein is from a sample of urinary or vaginal fluid, modification of the substrate indicates that the microorganism is present in the sample, while the absence of modification of the substrate indicates that the particular bacteria is not present in the sample.

In one embodiment, the sensor is incorporated in a lateral flow device or a membrane filtration device where, for example, the beads are trapped on one side of the membrane and, upon hydrolytic release, the dye or enzyme passes through the membrane to allow for a visible color change, e.g., reaction with a substrate.

Lateral Flow

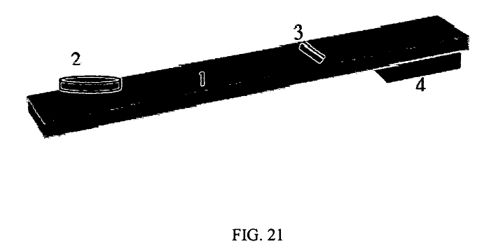

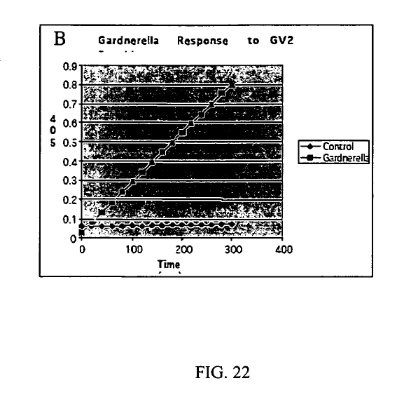

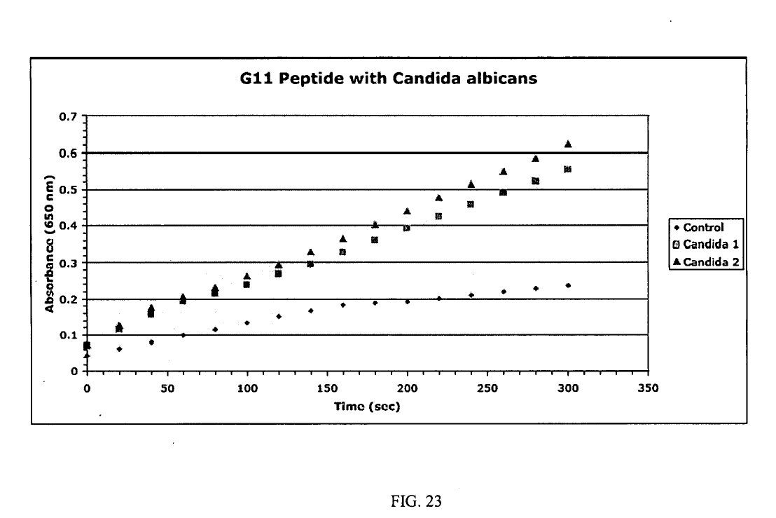

In addition to liquid phase assays, a lateral flow format provides a simple and rapid point-of-care diagnostic for vaginal infections. In one embodiment, as shown in FIG. 21, the device can have four components: a lateral flow strip (1), a conjugate membrane (2), a substrate line (3), and a wicking pad (4). The conjugate membrane will likely be a glass fiber (e.g., microfiber) membrane and can be printed with, for example, the HRP-peptide beads. The glass microfiber slows the flow of the liquid, thereby allowing time for the enzyme (e.g., microbial protease) to react with the HRP-peptide beads. The lateral flow membrane transfers the released HRP to the printed HRP substrate (e.g., naphthol) and the wicking pad at the back of the device acts as a sink to drive the liquid flow through the device. As the HRP reaches the naphthol substrate, the substrate turns blue and forms a line on the lateral flow membrane. The naphthol substrate can slowly diffuse, preventing the formation of a very distinct line. Dissolving the naphthol in a glue-like material or a material used for lamination, e.g., laminate, such as Colloidon (2% nitro-cellulose in amyl acetate), is sufficient to keep the line in place. Multiple sensors can be incorporated into one device.

In one embodiment, three diagnostic sensors can be incorporated into one device for the leading vaginitis pathogens associated with BV, CV, and TRIC. It is possible to print the materials on the same strip and separate the chemistries by forming channels. Alternatively, each chemistry can be printed on a separate lateral flow membrane and then the three strips can be laminated together in the final device.

The naphthol can be applied directly to the conjugate pad or applied to a transparent material which is applied to the pad. In one embodiment, the naphthol (dissolved in ethanol) is applied directly to the lateral flow membrane toward the top (north) end. The lateral flow test strips are constructed using a Matrix 2210 Laminator. A Porex K membrane is placed on an adhesive backed card. Two other pads, absorbent and glass fiber conjugate, are then adhered at the top and base, respectively, of the new membrane card. The absorbent pad is applied toward the naphthol end. TRISACRYL®-HRP-peptide conjugate beads are lined down on the upper portion of the glass fiber conjugate pad. When a bacterium is introduced to the glass fiber pad, it reacts with the conjugated beads, HRP is released and will flow laterally up the membrane and will interact with the naphthol, causing a color change to occur.

In another embodiment, the naphthol is applied to a transparent material, such as a polymer strip (e.g., acetate or laminate), which can be applied face down on the surface of the lateral flow membrane. The lateral flow test strips can be constructed using a Matrix 2210 Laminator. A Porex K membrane is placed on an adhesive backed card. Two other pads, an absorbent pad and a glass fiber conjugate, are then adhered at the top and base, respectively, of the new membrane card. TRISACRYL®-HRP-peptide conjugate beads are lined down on the upper portion of the glass fiber conjugate pad. Naphthol (dissolved in ethanol) is lined on the adhesive side of a strip of laminate toward the top end. When dried, this laminate is placed directly on the lateral flow strip with the naphthol end toward the absorbent pad. It is imperative that the start of the laminate be placed atop the portion of the glass fiber conjugate pad containing the line of beads. When a bacterium is introduced to the glass fiber pad, it reacts with the conjugated beads, the HRP is released and it will flow laterally up the membrane and will interact with the naphthol, causing a color change to occur.

In some embodiments, a 0.1% xanthan (xanthum) gum can also be applied to the conjugate pad. The gum keeps the beads in suspension. A medical-grade adhesive, paste, bond or glue can be placed on the upper portion, downstream of the conjugate pad atop the portion of the glass fiber conjugate pad containing the line of beads. This slows the rate of migration and allows for sufficient time for the peptide substrate to be hydrolyzed.

### EXAMPLES

The present invention will now be illustrated by the following Examples, which are not intended to be limiting in any way.

### Example 1

### Library Construction

A library of peptides was constructed, each member of the library comprising a random amino acid sequence, an epitope (affinity) tag, and a reactive side group(s) (e.g., one or more primary amines or cysteine groups) for chemical conjugation to a reporter enzyme or dye. Examples of suitable epitope tags include polyhistidine (His) and dihydrofolate reductase (DHFR, FolA). DHFR is coded for by the folA gene.

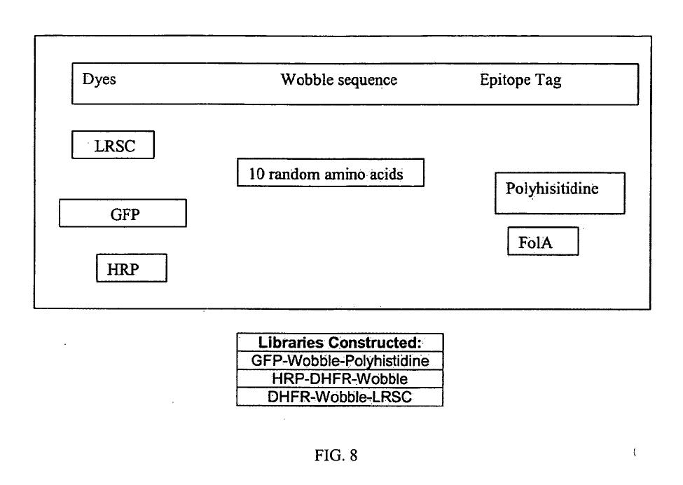

FIG. 8 illustrates construction of three peptide libraries using epitope tags (polyhistidine and FolA), dyes (horseradish peroxidase (HRP), green fluorescent protein (GFP), and lissamine rhodamine sulfonyl chloride (LRSC)), and sequences of 10 random amino acids (i.e., “wobble sequences”).

In some cases, a reporter enzyme can be cloned into the construct to circumvent having to attach chemically a reporter enzyme to the peptide. An enzyme used in this particular screen for BV targets was green fluorescent protein attached to a random peptide with a polyhistidine tag (GFP-random amino acid sequence-polyhistidine). A second example includes horseradish peroxidase-dihydrofolate reductase peptide chimeric protein (HRP-DHFR-random amino acid sequence) that uses the FolA portion to bind an affinity resin (e.g., methotrexate agarose) and HRP to produce a color change in the presence of 1 mM hydrogen peroxide and ABTS substrate. In a third example, the FolA random peptide sequence was conjugated to lissamine rhodamine sulfonyl chloride (FolA-random amino acid sequence-LRSC).

Briefly, a forward oligo primer was synthesized with a NdeI site adjoining 30 random nucleotides (corresponding to 10 random amino acids) upstream of a N-terminal portion of a reporter enzyme (GFP) and a reverse primer with a Xho I restriction site and the C- terminal portion of the protein. Once the gene sequences were amplified by polymerase chain reaction (PCR), the DNA was purified by the alkaline lysis method and ligated with T4 DNA ligase into an E. coli expression vector such as pET28 N-terminal (his tag) or pET24 (no tag). Upon electroporation of the DNA into E. coli (BL21 DE3), the cells were then incubated in SOC medium to allow for recovery and then plated onto LB plates with 30 μg/ml of kanamycin. After a period of time (e.g., overnight) the colonies expressing the GFP in frame glowed in the presence of UV light (365 nm).

The glowing colonies were patch plated onto a master plate and grown in 1 ml of LB in a micro-titer plate format (e.g., a plate having 96 wells) at 37° C. for overnight. An aliquot of the cells was diluted with glycerol to a final of 10%, flash frozen in liquid nitrogen and then stored in an −80° C. freezer until use (also know as Library colony freezer stocks). The remainder of the cells were induced with IPTG (final 1 mM) and then incubated for two hours at 37° C. to induce protein expression.

One or more members of the kanamycin/neo family of genes (Tn5, Tn903, and pJHl) can be positioned in the construct (e.g., downstream of the random amino acid sequence) to select for positive clones that do not have early termination in the random sequence (e.g., an in-frame stop codon sequence such as TAA, TAG, or TGA within the 10 amino acid random sequence).

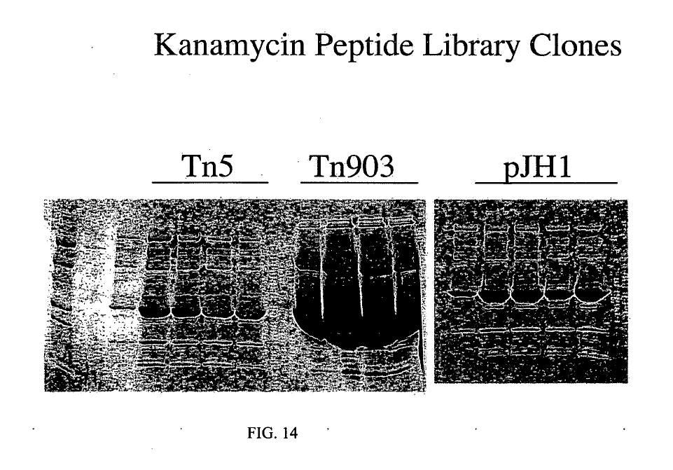

FIG. 14 is a SDS PAGE gel of three different polypeptide libraries consisting of kanamycin genes (Tn5, Tn903, and pJH1) with a polyhistidine tag and random polypeptide sequence (10 amino acids in length). The major band expressed from each of the clones is the kanamycin fusion protein. The Tn903 kanamycin library has the highest expression levels of the three constructs. By growing the colonies on kanamycin plates, it is possible to select for clones that have the proper reading frame within the 10 amino acid random sequence.

### Example 2

### Preparation of Cell Extracts

Following induction with IPTG, the cells were centrifuged into a pellet and then incubated with 0.2 mg/ml lysozyme (100 mM sodium borate pH 8.0, 500 mM NaCl) for 30 min at room temperature. The cells were frozen in liquid nitrogen and then immediately thawed at 37° C. three times to get efficient lysis of the bacteria. The thawed cells were then treated with 2 mg/ml DNAse I (10 Pipes, 150 mM NaCl, 10 mM MgCl₂) to lyse the DNA at 4° C. for 60 minutes in order to make the solution less viscous.

The samples were then incubated with beads that bind the epitope tag (e.g., 10 μl of NTA-resin to bind a polyhistidine tag or 10 μl of methotrexate agarose to bind the FolA tag). The sample was then placed into a microtiter filtration plate and washed three times to remove the unbound proteins. The fluorescent signal of the third wash was measured in a fluorescent microplate reader at an excitation of 355 nm and an emission of 510 nm (for GFP). Alternatively, the HRP activity can be measured in a microplate colorimeter at 405 nm with H₂O₂ and ABTS substrate. This background reading from the third rinse can later be subtracted from the signal of the released GFP (or HRP) peptides by extracellular bacterial proteases.

The activity of the protease was maintained by purifying the proteins quickly in the presence of 2M ammonium sulfate (NH₂)₄SO₄.2M (NH₂)₄SO₄ was used throughout the entire purification and the fractions that have activity were discerned by incubating an aliquot of each fraction with the HRP-peptide bead formulations in a spin filter assay. Although ammonium sulfate was used to “salt out” or precipitate proteins, in this case, the concentration (2M) of ammonium sulfate was used to prevent autolysis of proteases during the purification. Protein was purified with a Biologic DuoFlow Medium Pressure Liquid Chromatography Workstation by gel filtration (Superdex 75) and hydrophobic interaction chromatograph (HIC) eluted with 2-0 M gradient of (NH₂)₄SO₄.

### Example 3

### Labeling of Peptides with LRSC

For the FolA-random amino acid sequence peptide library, the beads can first be loaded with the unlabeled peptides, and after the third wash the peptides can be labeled with lissamine rhodamine sulfonyl chloride (LRSC) for 1 hour at room temperature in conjugation buffer (100 mM carbonate buffer, pH 9). The beads can be rinsed three more times to remove the unbound LRSC and then processed with microorganism extracts as described below.

### Example 4

### Determining Protein Activity

To determine if the protein has been secreted in an active form, a sample of the microorganism culture can be provided with chosen potential substrates and cleavage of these substrates can be determined. This can be done, for example, by combining the microorganism that produces the protein with the substrate in the appropriate media and incubating at ˜37° C. with gentle shaking. At preset times (e.g., ˜0.1, ˜0.3, ˜1.0, ˜3.0, ˜5.0, ˜24, and ˜48 hours) the samples are centrifuged to spin down the microorganism, and a small aliquot is removed for a SDS-PAGE gel sample. After completion of the time course, the samples are run on about a 10-15% gradient SDS-PAGE minigel. Then, the proteins are transferred to Immobilon Pseq (Transfer buffer, ˜10% CAPS, ˜10% methanol pH˜11.0, ˜15 V for ˜30 minutes) using a Bio-Rad semi-dry transblotting apparatus. Following transfer of the proteins, the blot is stained with Coomassie blue R-250 (˜0.25% Coomassie Brilliant Blue R-250, ˜50% methanol, ˜10% acetic acid) and destained (high destain for ˜5 minutes, ˜50% methanol, ˜10% acetic acid; low destain until complete, ˜10% methanol, ˜10% acetic acid) followed by sequencing from the N-terminal. Alternatively, the samples can be run on a mass spectrometer in order to map the sites of cleavage.

### Example 5

### Screening: High Throughput Screen

General Strategy

In order to develop a diagnostic for the causative agents of the female conditions described herein, a high throughput screen to identify peptide substrates for the specific proteases produced by the pathogens can be performed. Targets that are identified from the primary screen can be counter screened against other microorganisms and simulated vaginal fluids to confirm they are indeed specific and selective for the pathogen. Only those clones that do not cross-react with the panel of microorganisms and simulated vaginal fluids are sequenced. Following DNA sequencing of the clones to identify the amino acid composition, the peptides can then be designed and synthesized. Once a peptide is synthesized, it can be conjugated to a calorimetric component.

Clones of green fluorescent protein (GFP) tagged conjugates were used to identify targets in a high throughput screen. Each clone contained a plasmid harboring a gene that contains a polyhistidine tag (6 his), 30 random nucleotides (corresponding to 10 random amino acids), and green fluorescent protein (GFP). On the day before a screen, 864 glowing clones are grown up overnight at 37° C. in deep well microtiter plates (1 ml/well). In the morning, the cultures can be induced with 1 mM IPTG for 1 hour, the cells can be centrifuged for five minutes, and then the media can be decanted. Lysozyme (2 mg/ml) can be added and the cells can be flash frozen 3 times. The samples can be treated with 100 ul/well of DNAse I at 37° C. for 30 minutes to reduce the viscosity by digesting the DNA. An aliquot of the sample can then be transferred to a microtiter filter plate containing 10 μl of nickel-NTA resin. The pipeting of the reagents (IPTG, lysozyme, DNAse I, and beads) can be performed with a Tecan Genesis RSP 100 Robot with Gemini version 4.0 software.

The plates can be filtered by centrifugation three times with PBS to remove unbound material and then the GFP-peptide-bound bead can be incubated with microbial extract for 20 min at 37° C. The sample can be filtered and the flow through fluorescence can be compared with the background from the prior wash. If the microbe has a protease that finds the ideal target, then the release of GFP fluorescence from the bead can be over 100 times greater than the background.

Fluorescence can be measured on a Fluoroskan II instrument with an excitation filter centered at 380 nm and emission filter centered at 538 nm. Final wash, protease-liberated GFP and EDTA-liberated GFP in the well can be measured for each clone, as well as background control fluorescence from multiple wells containing diluted microbial supernatants exposed to mock clones and NTA resin.

Data analysis can be performed after subtracting the auto-fluorescence signals associated with the pathogenic microbe supernatants. Since expression of GFP varies with each well in a plate and can be dependent on a number of factors, release of GFP by proteases can be weighted relative to the total amount of GFP in the well and hits are assigned as percentage of total GFP released. Factors of variability in the screen include specific clone sequence, growth conditions, and variability in processing. Susceptible clones can be released at greater than 10-20% of the total GFP. Wells with unusually strong final wash values or with very low total GFP in the well are discarded. Hits are then “cherry-picked” and retested in a new screen to provide validation and can be counter-screened against commensal bacterial strains or specific proteases to assay specificity. Confirmed hits are then re-grown from the frozen stocks and plasmid DNA will be purified by the alkaline lysis method. The DNA can then be sequenced.

### Example 6

### Counter Screening: Specificity and Sensitivity of Peptides

In Vitro Experiments

Once primary targets from the HTS have been identified, the positive colonies can be grown up into a new deep well microtiter plate and then extracts can be screened with bacteria to confirm that they do not cross react with the primary targets. The sensitivity and specificity of the peptide targets, as described herein, in artificial vaginal fluids can be determined with clinically relevant levels of pathogens (10⁶ CFU/ml G. vaginalis, 10⁵ protists/ml T. vaginalis, and 10⁶ CFU/ml filamentous infectious C. alibicans). The best medium to use for these studies is a chemically defined medium (CDM) described by Geshnizgani, A. M. et al., “Defined Medium Simulating Genital Tract Secretions for Growth of Vaginal Microflora,” J Clin Microbiol, 30:1323-1326 (1992). The CDM is unusual in that it is able to support growth of a complex array of microorganisms found in vaginal fluids. Optimal growth in the CDM is obtained in approximately 13 hours. The sensor, as described herein, for Gardnerella vaginalis detects 10⁸ cfu/ml in five minutes.

Sensitivity can be measured by performing dilution series of each pathogen to determine the minimal colony forming units/ml that gives a reproducible color change. Sensors, as described herein, have the sensitivity to detect the presence of infection levels of bacteria (10¹⁰ cfu/ml), but do not detect the inconsequential amounts of G. vaginalis that are found in normal fluids (10⁵ cfu/ml).

Specificity can be determined by challenging the sensors with vaginal microorganisms, for example, G. vaginalis, L. acidophilus, T. vaginalis, and C. alibicans, in addition to other bacteria associated with BV such as Bacteriodes spp., Mobilincus spp., Mycoplasma hominis, E. Coli, Peptostreptococcus spp., Prevotella bivia, and Porphyromonas spp. Sensors for G. vaginalis, L. acidophilus, and C. albicans should be specific and should not cross react with each other. The color released from the sensors can be read in a Molecular Devices microplate reader with Softmax Pro Kinetic software.

The specificity and sensitivity of the HRP or dye labeled sensors can be tested in vitro using cultured clinical strains. Clinical strains of C. albicans, T. vaginalis, L. acidophilus, and G. vaginalis can be obtained and grown overnight in a suitable medium, described above. The concentration of each culture can be estimated by dilution with the medium, followed by measurement of absorbance at 600 nm. The bacterial concentration can be calculated using an appropriate calibration curve, and serial dilutions of all four strains can be prepared. Sensor sensitivity can be evaluated by quantifying the dye released from the sensors after incubation at room temperature for 5 minutes with each of the serial dilutions.

Bacteriodes spp., Mobilincus spp., Mycoplasma hominis, Peptostreptococcus spp., Prevotella bivia, Porphyromonas spp, Candida albicans, E. coli, Trichomonas vaginalis, Gardnerella vaginalis, and Lactobacillus can be grown using methods recommended for each clinical isolate. Briefly Candida albicans can be grown at room temperature without agitation in yeast malt broth (YMB Sigma 3752). T. vaginalis can be grown at 35° C. in LYI Entamoeba medium. Gardnerella vaginalis will be grown with 5% CO2 at 37° C. for overnight in NYC III Medium (ATCC 1685). Lactobacillus acidophilus can be grown for 1-2 days 37° C. with 5% CO₂ in tomato juice, yeast extract, milk (Carolina HT-82-1439) medium. E. coli can be grown in M9 minimal medial at 37° C. overnight. All strains except those mentioned above require growth in an anerobic bell jar chamber that has been incubated with a gas pack to remove the oxygen from the container. Bacteriodes spp. and Prevotella bivia can be grown on brucella blood agar supplemented with vitamin K and hemin (BD 297848) and cultured in reinforced clostridial medium (BD 218081) for 2 days at 35° C. Mobilincus spp. can be grown on columbia agar (BD 211126) and cultured in Schaedler broth (BD 212191) for 1-2 days at 37° C. Peptostreptococcus spp. can be grown on trypticase soy agar with 5% sheep blood (BD 221239) and cultured in thioglycollate medium with calcium carbonate, enriched with vitamin K and hemin (BD 297264), at 35° C. for 2 days. Mycoplasma hominis can be grown on mycoplasma agar (BD 241210) supplemented with mycoplasma suppliment (BD 283610) and cultured in mycoplasma medium also supplemented at 37° C. 1-2 days. Lastly, Porphyromonas can be grown on chocolate agar with yeast extract, hemin, vitamin K (Remel 01318) and cultured in Todd-Hewitt broth (Difco 249240) supplemented with 10 ug of hemin/ml and lug of vitamin-K/ml for 2 days at 37° C.

Various bacteria were cultured overnight at 37° C. in NYC III media with 5% CO₂. A 10% slurry of Affigel-GV2-HRP in PBS was prepared, and 20 μl was added to six wells of a 96-well filter plate (Millipore Multi-screen HTS). Aliquots of the bacteria, including Gardnerella vaginalis, Pseudomonas aeruginosa, E. coli, Streptococcus pyogenes, Enterococcus faecalis, and Staphylococcus aureus, were added (100 μl) to the beads, and the plate was allowed to sit for 5 minutes at ambient temperature. The supernatants were centrifuged into a low-binding assay plate (Coming 3641), and assays for HRP were then run. 2,2′-azino-bis(3-ethylbenzthiazoline-6-sulfonic acid) (ABTS, US Biological) is a commonly used substrate for HRP, giving a water-soluble blue-green color in the presence of HRP and hydrogen peroxide. The formation of the bluegreen color can be followed at 405 nm, with higher slopes indicating the presence of higher amounts of HRP. The GV2 peptide was found to be quite specific for G. vaginalis, with a small amount of signal from E. coli.

Ex Vivo Testing: Clinical Testing

It is necessary to demonstrate that the diagnostic sensors can not only work in vitro but can also specifically detect physiologically relevant levels of bacteria ex vivo from vaginal swabs from patients that have been diagnosed with a lower genital tract infections, such as female patients presting with symptoms of BV, CV, or TRIC. Vaginal swabs can be collected from women and female child patients that are presenting with symptoms for vaginitis. The swabs can be extracted with 1 ml of PBS. Vaginal fluids can be very proteinacious, viscous, and contain a complex mixture of components. In addition, the micro flora are equally complex and vaginal fluids can have 15 different microoganisms in a given swab. Due to the complexity of vaginal fluids, it is critical to appreciate that the virulence factors and proteases produced during infection may not express at the same levels found in bacterial culture medium. Polyester swabs can be used to obtain vaginal fluids because they do not bind protein or bacteria. In contrast, cotton swabs can retain as much as 50% of the protein and bacteria.

For clinical testing, validation of the sensors, as described herein, can be performed to determine if they are specific and correlate well with infection based on the gold standards for BV, CV, and TRIC. For each sample a wet mount microscopy (gram staining and Nugent test), protein assays, culturing, and HRP-peptide-bead and Simple Dye-Peptide-Bead assays and quantitative PCR (qPCR) can be performed.

Assays for Clinical Swabs

The samples can be collected and stored on ice prior to testing of the swabs for pathogens. The swab can be extracted for 5 min. on ice with 1 ml PBS with gentle swirling. Serial dilutions can be referenced in PBS and then the swab samples plated on blood agar and MacConkey plates. The microbes can be grown either at room temperature or 37° C. (as described above). The presence of BV can be ascertained by using the method of Nugent et al., infra. CV can be determined as described by Sobel et al., infra. The total number of T. vaginalis protozoa can be ascertained by wet mount microscopy. This will confirm that the diagnostic sensors are specific for the pathogen and do not crossreact with vaginal fluids. Vaginitis sensors can be tested with an aliquot of the fluid extracted from the vaginal swabs. Briefly, HRP-Peptide-beads can be pre-rinsed on the filter plate and then 25 μl of the extract, as well as the control, can be placed into a well on a 96-well plate containing 20 μl of HRP-Peptide-TRISACRYL® bead slurry (1:10 dilution of TRISACRYL®) and allowed to incubate with gentle agitation for 4 minutes. Samples can be transferred to a 0.2 μm filtration plate. At exactly 5 minutes, the samples can be spun down in a plate in a microplate centrifuge to remove the beads. TMB substrate (100 μl), 25 μl of the bead clip samples, and 75 μl PBS can be added to a fresh 96-well plate. The TMB response can be read immediately in the Molecular Devices plate reader at 650 nm for 300 seconds with 20-second intervals.

If the peptides do not meet the sensitivity criterion, the HRP to bead ratio can be changed or the number of beads per assay can be increased to increase the signal to noise ratio. If the peptide does not meet the 90% selectivity goal then the peptide sequence can be a random amino acid sequence to find a construct that is more specific. For some of the microbes it may be necessary to choose the optimal peptide from several candidates identified from the screen.

Swabs can also be tested for reactivity with L. acidophilus, G. vaginalis, T. vaginalis, C. albicans sensors. Colony counts can be used to identify yeast and the different morphologies. Clinical diagnosis can be used to assess correlation with the sensors. Traditional testing, such as use of the Nugent test to diagnose BV, can also be used to assess results.

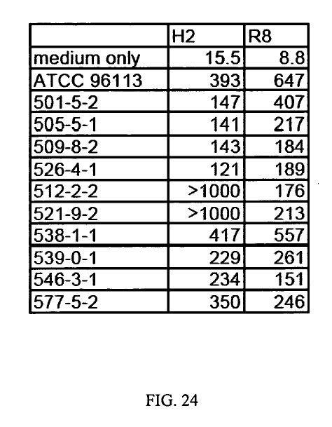

For example, ten clinical C. albicans isolates kindly provided by Dr. Andrew Onderdonk (Brigham and Women's Hospital) were tested alongside a strain obtained from C. albicans ATCC. The yeast cells were grown in YCB-BSA (23.4 g yeast carbon base, 2 g yeast extract, 10 g dextrose, and 5 g of BSA per liter—adjusted to pH 5.0). An aliquot of the cell-free growth medium was mixed with an HRP-peptide-Affigel conjugate for ten minutes and the amount of released HRP was determined. As indicated in FIG. 24, both the H2 (QKTTIKKLKH) (SEQ ID NO: 9) and R8 (KPKAFLKVGN) (SEQ ID NO: 6) peptides reacted strongly with all ten clinical isolates, giving a sensitivity of 100%. The R8 sequence H2N-HHHHHHKPKAFLKVGNC-OH includes a histidine tag (HHHHHH) that is not part of the original peptide sequence but is only used for immobilization of the peptide. An initial study of the specificity of these peptide conjugates showed that they were not significantly clipped by the other vaginal bacteria Gardnerella vaginalis, Lactobacillus acidophilus, and Escherichia coli.

For example, in one embodiment, the H2 peptide was conjugated to Blue dye #1 as a reporter on the C-terminal end. The N-terminal end was conjugated to Affigel 10 beads (BioRad). The beads were exposed to an aliquot of overnight grown Candida albicans for a period of 24 hours at 37° C. A control set of beads was also exposed to culture medium. Blue dye released from the beads by the action of Candida albicans was collected on a membrane to produce a clear signal. The SB6407 (Pall Life Sciences, Ann Arbor, Mich.) collection membranes included in the solution for 24 hours showed a highly visible blue color collected from the Candida albicans solution and no signal from the control medium.

### Example 7

### Identification of Novel BV Targets

Upon washing the GFP, HRP or LRSC coated beads, each well can be incubated with microbial supernatants of Lactobacillus, Gardnerella vaginalis (or other BV symptomatic bacteria, such as Bacteriodes sp., Mobilincus spp., Peptostreptococcus spp., Mycoplasma hominis, Prevotella bivia, and Porphyromonas spp.) or protozoa (e.g., Trichomonas vaginalis) for 30 min at 37° C. prior to centrifuging into a fresh micro-titer plate to collect the released peptide-GFP or peptide-dye conjugates. Typically, about 100 plates (˜10,000 clones) are screened to identify strong positive signals from the peptide library.

Measurement of Protease Activity

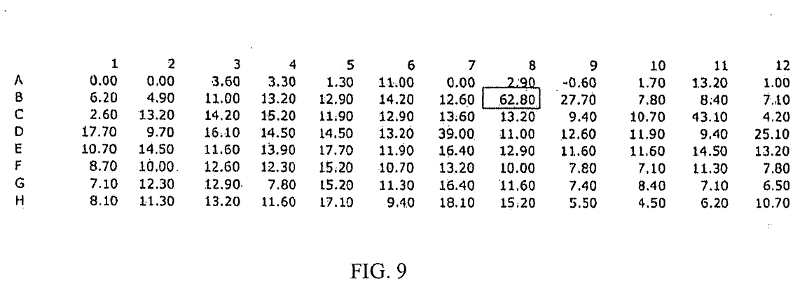

Wells with strong fluorescent or color signals turn out to be putative peptide targets for proteases from the respective bacteria. The GFP-random amino acid sequence-polyhistidine library was successfully used to identify novel targets for Lactobacillus sp. FIG. 9 illustrates the signal strength measured in the various wells of plate number 69. Well B8 of plate number 69 had a strong signal (62.8) when exposed to Lactobacillus. Many of the wells in the plate had very little GFP-peptide released from the beads, suggesting that Lactobacillus sp. makes a protease that is specific.

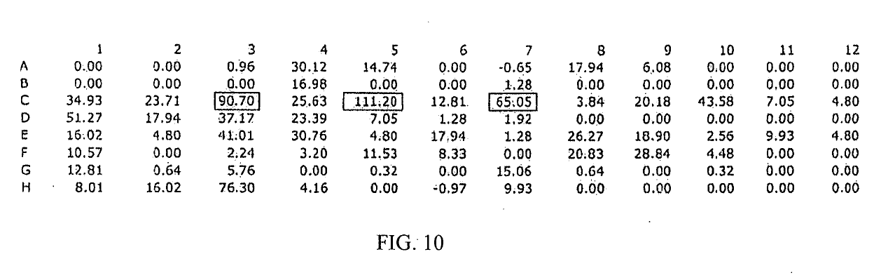

The GFP-random amino acid sequence-polyhistidine library was also used successfully to identify novel targets for Gardnerella vaginalis. FIG. 10 illustrates the signal strength measures in the various wells of plate number 76. Plate 76 had strong fluorescence in wells C3, C5 and C7 after exposure to Gardnerella extracts. However, wells C5 and C7 were previously determined to cross react nonspecifically with other common pathogens, such as Staphylococcus aureus and Pseudomonas aeruginosa, and, therefore, were dropped from the secondary screen.

Synthesis and Conjugation

The positive clones for Lactobacillus acidophilus (plate 69, well B8) and Gardnerella vaginalis (plate 76, well C3) were DNA sequenced using an ABI 377 Prism DNA sequencer to identify the genetic composition. The translated amino acid sequences for the two peptides are shown below.

| Plate/ | | |
|---|---|---|
| Well | Target | Amino Acid Sequence |
| 69/B8 | Gardnerella | PFINETYAKFC (SEQ ID NO:1) |
| 76/C3 | Lactobacillus | ITTTSSKHEHC (SEQ ID NO:2) |

The two putative peptides were synthesized, coupled to Affigel beads (Bio-Rad, Hercules Calif.), and then labeled with horseradish peroxidase in the following manner:

Peptides were coupled to Affigel beads as described by the manufacturer. Similar results were obtained using small latex beads with carboxylic acid groups. Briefly, 1 mg of peptide was incubated with 1 ml of affigel in conjugation buffer (phosphate buffered saline) for 1-2 hours at room temperature. For the small latex beads functionalized with carboxylic acids, the peptides/proteins are coupled with EDC (1-ethyl-3-[3 dimethylaminopropyl] carbodiimide hydrochloride). The beads are either used immediately or conjugated further with HRP-maleimide or stored at 4° C. under a cushion of 2% trehalose or 2% sucrose.

HRP is conjugated to sulfo-SMCC though a reactive primary amine group. The reaction is performed for 1 hour in the presence of 50 mM sodium phosphate buffer (pH 7.4). The functionalized HRP is removed from the unbound sulfo-SMCC by gel filtration using P6 Biogel P polyacrylamide beads, Biorad (Hercules, Calif.) or Sephadex G50 beads (Amersham Biosciences, Piscataway, N.J.). The HRP-maleimide elutes in the void volume, whereas the free sulfo-SMCC elutes very slowly from the column. The HRP binds hemin, and, thus, has a brown color that can be used to quickly identify the void volume containing the HRP-maleimide. The HRP-maleimide is either lyophilized or used immediately in the conjugation with peptides.

Peptides in solution or attached to Affigel beads are treated with 1 mM dithiothreitol (DTT) prior to reacting with the HRP-maleimide. The DTT is removed by dialysis, gel filtration, or, in the case of peptides coupled to beads, by 1000-5000 rpm centrifugation for 5 minutes.

Gardnerella vaginalis was detected with the peptide PFINETYAKFC (SEQ ID NO: 1) which was conjugated to HRP. The peptide became hydrolyzed from the beads and was visualized by spin filtering the beads and incubating an aliquot of the filtrate with H₂O₂ and ABTS substrate. A color change was observed quickly in the tube containing Gardnerella vaginalis but not in the test tube. Alternatively, the color change can be measured on a plate reader at 405 nm.

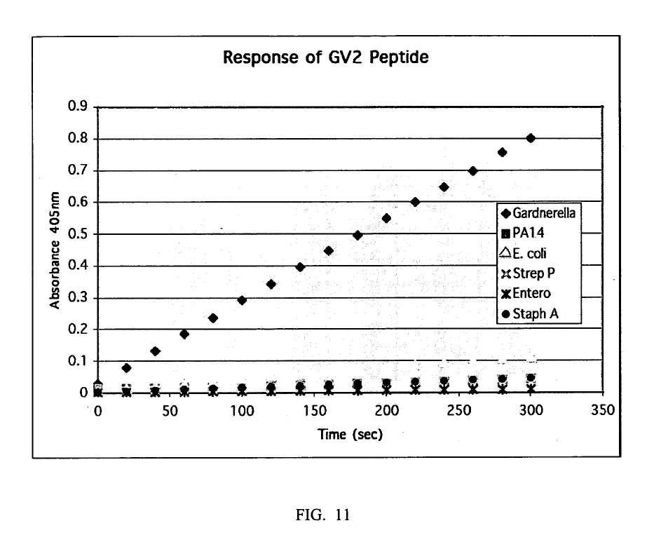

FIG. 11 illustrates the specificity of the GV2 (PFINETYAKFC) (SEQ ID NO: 1) peptide for Gardnerella vaginalis over the course of five minutes when incubated with Gardnerella vaginalis, Staphylococcus aureus, Streptococcus pyogenes, Escherichia coli (E. coli), Pseudomonas aeruginosa, and Enterococcus faecalis. The extracts were filtered through microtiter plate filters and the HRP release was measured at 405 nm with ABTS substrate in the presence of hydrogen peroxide (H202). The peptide turns blue in the presence of Gardnerella but not in the presence of other common pathogens, indicating its specificity for Gardnerella vaginalis.

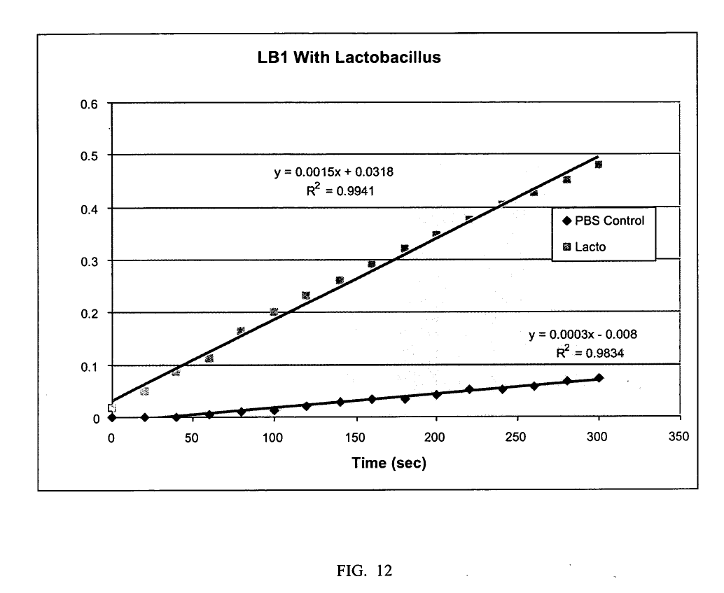

FIG. 12 is a graph showing that the peptide ITTTSSKHEHC (SEQ ID NO: 2) detects an unknown protease from Lactobacillus.

Similarly, a number of peptide targets for C. albicans have been identified: KPSIKPTPPY (SEQ ID NO: 8), the sequence QKTTIKKLKH (H2) (SEQ ID NO: 9), the sequence KPKAFLKVGN (R8) (SEQ ID NO: 6), the sequence TPIQIHTILH (SEQ ID NO: 10), the sequence INLSKKQIYP (SEQ ID NO: 11), the sequence LYPSQNPVIK (SEQ ID NO: 12), and the sequence NITKKSTKII (SEQ ID NO: 13).

The sequence, known as H2, QKTTIKKLKH (SEQ ID NO: 9) was modified (substitution of lysines (K)) resulting in the sequence QRTTIRRLRH (SEQ ID NO: 21). Amino acid groups such as cysteine can be added to the sequence to allow attachment of dyes or enzymes used as reporters.

For example, a specific peptide (G11, which is TPIQIHTILH) (SEQ ID NO: 10), which is able to detect C. albicans, was grown in artificial vaginal fluid overnight at 37° C. It was found that the artificial vaginal fluid had some background signal that needed to be removed by washing the beads extensively with detergent (0.1% TRITON®-X100) to prevent nonspecific sticking of the peptide dye conjugate.

Peptide substrates for proteases produced by the vaginal pathogen Trichomonas vaginalis were also identified. This microbe was grown in LYI entamoeba medium overnight and the cell-free growth medium was used in the high throughput screening of 4,900 clones from a GFP-random peptide fusion library.

Seven peptides detect Trichomonas vaginalis were identified: NNPLPKIQKN (38H9/T7) (SEQ ID NO: 14), KNPKLQDHYI (44B5/T7) (SEQ ID NO: 15), QINKALKQPK (41El 1/T7) (SEQ ID NO: 16), QIPKSLHPIT (42D3/T7) (SEQ ID NO: 17), LHNYVLLRNIL (38H8/T7) (SEQ ID NO: 18), SKQQDIIKKY (44E6/T7) (SEQ ID NO: 19), NKTNKTKHAY (42H8/T7) (SEQ ID NO: 20). Peptides 39H9 and 42D3 were altered to remove the lysine residues prior to peptide synthesis. The peptides were conjugated to HRP and affigel 10 beads and produced a visible color signal in five minutes at 650 nm when an aliquot of the protease-treated product was incubated with tetramethyl benzidine.

### Example 8

### Stability Studies for Simple Blue Dye-Beads

In order to study the stability of simple blue-dye beads, simple blue dye-peptide beads are printed onto a 1 cm×1 cm section of non-woven material from a feminine pad. A 50 μl sample of beads (2.5 mg/ml stock) can be printed in a line onto each non-woven material sample using a Biodot XYZ dispenser platform with AIRJET QUANTI DISPENSER®. The samples will be dried at 40° C. in the convection oven for 2 hours. Timepoints can be tested in triplicate for activity including controls. Six membranes can be made for each time point. One set of membranes can be tested at time zero (after drying and prior to the accelerated stability study) using the protocol below. The other sets of membranes can be placed in a convection oven at 40° C. for 3 months. Once a week a set of six membranes can be tested using the following protocol: each of the six membranes is placed in a 2 ml siliconized eppendorf tube. Three tubes can serve as controls and 200 μl of PBS can be added. The other three tubes can have 100 μl of PBS plus 100 μl of overnight grown bacteria (corresponding to the peptide of interest) and reacted for 60 minutes. After the reaction time, all samples can be spun through a 0.22 μm spin filter plate (Millipore Multiscreen HTS) to remove the beads. The supernatants can then be analyzed for dye release by placing a portion of the supernatant in PBS and reading the absorbance at approximately 408 nm (1st peak) or 630 nm (2nd peak). The peak absorbance can be verified on the blue dye #1 maleimide by scanning the absorbance over the visible spectrum. As a start, 50 μl of supernatant can be added to 450 μl PBS. The amount of supernatant may need to be adjusted up or down depending on the strength of the response. This can be determined at time zero and used for the entire study. Samples can be compared by averaging the absorbance value for the triplicates of the bacteria. The average absorbance for bacteria can be corrected for background by subtracting the average PBS control absorbance. The average absorbance for bacteria can then be compared over the 3 month time period to the time zero sample using Student's T-test for significance.

### Example 9

### Quantiative Criteria for Sensors

In the identification of specific peptide targets for T. vaginalis, C. albicans, G. vaginalis, and L acidophilus, one goal is to have each peptide target, as described herein, detect (produces a signal to noise ratio of 10 or higher) a clinically relevant concentration of the corresponding pathogen in artificial vaginal fluid (10¹⁰ cfu/ml G. vaginalis, 10¹⁰ cfu/ml L. acidophilus, 10⁵ protist/ml T. vaginalis, and 10⁵ cfu/ml C. albicans) for 90% or greater of the 20 clinical isolates of each species tested. In addition, the specific peptide targets preferably must not show cross-reactivity with 90% or greater than clinically relevant levels of other microbes that are commonly found in vaginal fluids and the 3 other pathogens of interest. This is true, for example, for both the HRP-peptide-bead construct and the blue dye #1-peptide bead construct. Quantitative PCR (qPCR) is a highly sensitive method used to quantitate unknown samples. The fluorescent dye used is SYBR Green and this dye is measured in real-time as the PCR products accumulate after each cycle. qPCR uses a standard curve of numbers which will specify the amplification quantity and unknown samples are thus measured against it. Following the completion of 40 cycles a quantitative number is given, representing the amount of copies amplified for each unknown sample provided it falls within the range of the standard curve. The qPCR process is a highly preferred alternative to traditional PCR methods for its specific determination of copies produced.

For the incorporation of the enzyme sensor technology into a lateral flow POC device, preferably, the sensitivity should be greater than 92% and the selectivity should be greater than 90% for each pathogen corresponding to the sensitivity and selectivity found from the targets in the HRP-peptide bead format. The HRP-peptide beads on the glass conjugate pad should retain ≧90% of their activity over the 3-month period. An excess of beads in the POC device can be used to ensure robust line development (1 ng HRP and greater) even with slight losses in activity over time. The ex-vivo clinical study with swab samples from patients with vaginitis should show a sensitivity and selectivity of greater than 85% corresponding to the clinical microbiological and PCR results.

### Example 10

### Food Grade Sensors for Consumer Products

Food grade sensors can be used in consumer products. For example, food grade Blue Dye #1 derivatized with a maleimide group was attached to the modified H2 peptide (QRTTIRRLRH) (SEQ ID NO: 21) and then conjugated to Affigel 10 agarose beads (BioRad (Hercules, Calif.)). The resulting beads were incubated with growth medium (1:2 BHI/PBS) alone (control) and an overnight grow-up of Candida albicans for 24 hours at 37° C. In the sample with Candida albicans, the peptide was hydrolyzed and the blue color migrated and collected into a membrane that is in close proximity of the release membrane. The preferred ion exchange membranes to collect the free dye are SB6407 and Biodyne B (Pall Life Sciences, Ann Arbor, Mich. and positively charged PVDF (Millipore)). These membranes have strong positively-charged quaternary ammonium groups. In contrast, uncharged, hydrophobic and negatively charged membranes such as nylon, ICE, p4, 3M C8, 3M C 18, and Biodyne C have negligible binding capacity for the free blue dye. Visible blue dye release can be seen in approximately 3 hours resulting in collection of color in a feminine pad.

In one example, dye-conjugated Affigel 10 beads were exposed to Candida albicans supernatant (20 μl of 1:10 dilution of beads) or a medium control (1:2 BHI/PBS) for 24 hours. The color was collected with a positively charged membrane (SB6407) in with the bead reaction. The results demonstrated that the control membrane, which was treated with 1:2 BHI media alone, did not release the color from the Affigel 10 beads. In contrast, the active protease hydrolyzed the modified H2 peptide (QRTTIRRLRH) (SEQ ID NO: 21), thereby releasing color into the collection membrane. Similar results have been obtained with TRISACRYL® beads.

### Example 11

### Synthesis and Characterization of Sensors

Conjugation of the Peptide to the Colorimetric Compound

The speed of the diagnostic assay required dictates the choice of colorimetric component to conjugate to the peptide. An enzyme reporter such as HRP gives a rapid color change in 5 minutes, whereas a simple dye is limited by diffusion and takes time to collect the color (˜30 minutes-1 hour). The enzyme typically used is horseradish peroxidase when time is of the essence and a conjugated food grade blue dye #1 when safety and simplicity are primary concerns. For the conjugation of HRP a three-step process can be used: 1) Labeling HRP with sulfosuccinimidyl-4-(N-maleimidomethyl)cyclohexane-1-carboxylate (SMCC) in phosphate buffered saline (PBS) to produce a maleimide group on the HRP; 2) Conjugation of HRP maleimide to a cysteine at the C-terminus of the peptide in phosphate buffer with 5 mM EDTA pH 7.5; 3) Coupling the HRP-peptide to small latex beads in MES buffer with 1 mM 1-ethyl-3-(3 -dimethylaminopropyl)carbodiimide hydrochloride (EDC). The coupling of the HRP peptide to the microbeads can be performed with the crosslinker EDC in MES buffer that conjugates the carboxyl groups on the bead to the amino terminus of the peptide. In the case of the slower reacting food grade dye, blue dye #1 with a maleimide group can be synthesized to conjugate directly to the cysteine at the C-terminus of the peptide.

The peptides can be synthesized using Fmoc chemistry. Typically, HRP (Roche) can be conjugated to SMCC using a 2-fold molar excess of HRP to cross-linker. Following coupling of the HRP to the maleimide, the free SMCC can be removed by a 10 ml desalting column (PD10, Amersham). The purified HRP maleimide can then be reacted with peptide for 8 hrs. in phosphate buffer pH 7.5. The HRP peptide conjugate can be purified once again over a gel filtration column to remove the un-reacted peptide. The HRP-peptide conjugate can then be cross-linked to microbead particles using conventional EDC chemistry. Carboxy methyl TRISACRYL® beads (Pall) can be used with a 30-80 μm size range for conjugation. Briefly, 1 mM of EDC can be added to the peptide and then TRISACRYL® beads are added at a final concentration of 100 mg/ml. The HRP-peptide-bead conjugate can be washed three times in PBS in order to remove the nonspecifically bound material. It can be advantageous to rinse with surfactants such as TRITON®-X100 and TWEEN® 20 with the caveat that long-term storage of HRP with detergents can be detrimental to enzyme activity. Although several peptides can be conjugated and tested in vitro with common vaginal microorganisms, it is quite manageable to label, purify, and rinse 6-8 peptides simultaneously within a 2-day period. In some instances, the peptides can be rather insoluble, in which case the peptides can be suspended in dimethyl sulfoxide (DMSO) prior to conjugation to HRP.

Once the peptides are conjugated they can be first tested with the target microorganism at the levels that are indicative of a vaginal infections (G. vaginalis 10¹⁰ cfu/ml, T. vaginalis 10⁵ cfu/ml, C. albicans 10⁵ cfu/ml). 20 clinical isolates can be tested for each of the target microorganism and at least 2 of each of the less common BV-associated microorganisms with vaginal fluids including Bacteriodes spp., Mobilincus spp., Mycoplasma hominis, Peptostreptococcus spp., Prevotella bivia, and Porphyromonas spp. Typical assay conditions include 100 μl of sample-containing bacteria placed in 90 μl of PBS. The mixture can be incubated with 10-20 μl of a HRP-peptide-bead slurry for 5 min. prior to filtration in a 1.5 ml microcentrifuge tube. An aliquot of the filtrate (e.g., 20 μl) can then be reacted with ABTS containing H₂O₂ and then read in a microplate reader at 405 nm.

Alternatively, the aliquot of bacteria can be diluted with PBS with 1 % H₂O₂ and then loaded onto the end of a lateral flow membrane that has been printed and air dried with the HRP-peptide-bead conjugate. A wicking pad at the end of the lateral flow membrane, as described herein, can be used to drive the flow of HRP in the direction of the colorimetric substrate. By using a slow flow lateral membrane, the bacterial extract has enough time to release the HRP from the beads over a five-minute period. The HRP that is released from the beads by the specific bacterial proteases is able to migrate through the tight pores of the membrane and react downstream with its substrate such as naphthol. A preferred way to lay down a permanent line of the naphthol substrate is to mix it with 2% nitrocellulose in methanol. The beads are too large to migrate down the membrane. The BV sensor can comprise three components, whereas the CV and TRIC sensors can include a single indicator for the presence of a specific protease. The BV sensor can show a minus sign for the presence of Lactobacillus at 10¹⁰ but a plus sign in the presence of an increase in pH>4.5 and increase in the presence of Gardnerella vaginalis. The pathogen specific proteases that recognize each peptide substrate can be identified.

The specific proteases that recognize the peptide substrates can be characterized. Proteases can be very difficult to purify due to autolysis. However, the problem of autolysis can be reduced by 1) keeping all materials on ice, 2) using 2M ammonium sulfate, and 3) using an automated chromatography system to purify the proteases in less that 8 hrs. Using this approach, proteases from, for example, Gardnerella vaginalis, Trichomonas vaginalis and Candida albicans can be purified and identified, as described herein, in less than 4 hrs. A combination of gel filtration (Superdex 75) and hydrophobic interaction chromatography can be used to purify unknown proteases from the bacteria. Sequencing 7-10 residues from the amino terminus is sufficient to identify the proteases using NCBI's protein-protein BLAST. Findings from purifying, assaying and getting N-terminal sequence from the proteases indicate that the Superdex 75 column in combination with the HIC column can be sufficient to isolate a single band on a SDS PAGE gel. The protein can be blotted to Immobilon PSEQ membranes for N-terminal protein sequencing.

The proteases from G. vaginalis, L. acidophilus, T. vaginalis, and C. albicans can be purified using a combination of gel filtration and hydrophobic interaction chromatography to purify the proteases that react with the peptide substrate. Briefly, 25 ml of an overnight group of the bacteria can be centrifuged and the supernatant 0.2 uM filtered to remove the microorganisms from the supernatant.

The supernatant can then be incubated with ammonium sulfate in two steps to precipitate proteins insoluble at 50% and 75% (NH₂)₄SO₄. Ammonium sulfate crystals can be slowly added to the bacterial supernatant in an ice bath. After 30 minutes the supernatant can be centrifuged and the pellet will be stored on ice while the supernatant will be further precipitated with ammonium sulfate at a final concentration of 75%. After incubating the supernatant with the 75% (NH₂)₄SO₄, the sample can be saved on ice and the supernatant can be placed in a new 15 ml conical centrifuge tube. The pellets can be resuspended in 500 μl of 10 mM Tris, pH 8.0 containing 150 mM (NH₂)₄SO₄ (resuspension buffer). An aliquot of the supernatant and two resuspended pellets can be tested for protease activity using the method described above. The sample that has the peak of protease activity can be further purified by gel filtration on a sephacryl s100 24 ml column equilibrated with 3 column volumes of the resuspension buffer. The column can be run at 1 ml/minute and the fractions can be collected in a volume of 0.5 ml.

The fractions of the gel filtration column can be tested for protease activity and the peak fractions will be adjusted to 2M (NH₂)₄SO₄ and then run over a 1 ml HIC column with a linear gradient of (NH₂)₄SO₄ from 2M-50 mM. By running a high-to-low salt gradient on the HIC column, the strength of the protein's hydrophobic interactions with the phenyl resin are reduced, thereby eluting the proteins from the column. Protein assays (Bio-Rad) with a set of bovine serum albumin standards can be performed as an internal control for the amount of protein extracted from each vaginal swab.

Real-time qPCR can be performed on an iCycler MyiQ single color detection system from Bio-Rad. The DNA standard can be diluted to the appropriate concentrations in water and 5 ul of each dilution can be used in a 25 ul reaction volume. The standard curves range from 5×10¹ to 5×10⁵ or 1×10² to 1×10⁶ and all standards are run in triplicate. The unknown samples are diluted 1:10 and 1:100 in sterile water and run in triplicate. A two-step plus melt curve can be run with 40 cycles. A master mix of the IQ SYBR green supermix as follows:

Components Volume per reaction

| | | |
|---|---|---|
| IQ SYBR green supermix | 12.5 | μl |
| Primer 1 | 2.5 | μl |
| Primer 2 | 2.5 | μl |
| Template | 5 | μl |
| Sterile water | 2.5 | μl |
| Total volume | 25 | μl |

T. vaginalis may not express proteases in LYI Entamoeba medium. An alternative strategy can be to grow the microorganisms, e.g., protozoa, in CDM medium which mimics the components of vaginal fluids, and therefore, is likely to express proteases. It may be necessary to run extracts of T. vaginalis on a zymogram gel in order to determine the total protease activity. If the proteins do not bind well to a HIC column, an ion exchange column can be substituted and a buffer used that is consistent with the standard rule of ion exchange chromatography, i.e., the buffer needs to be at least 1 pH unit above the isoelectric point of the protein to bind. In the case of unknowns it is often desirable to link a 1 ml quaternary amine (QAE anion exchange column) with a 1 ml sulfopropyl (S cation exchange column) to load the protease peak from the gel filtration column. Once the columns are rinsed until the UV absorbance approaches the baseline, then the columns can be separated and the proteins eluted from the QAE or S columns individually.

Stability: HRP-peptide Bead Constructs

Glass conjugate pads (1 cm×1 cm, Millipore) can be silanized by reaction with 2% 3-aminopropyl triexthoxysilane in acetone for 2 hours. Rinsing is two times with acetone, methanol, and then distilled water. The membranes can then be dried at 40° C. before use. The HRP-peptide-beads can be rinsed before placement on membranes. A 20 μl aliquot of bead stock (25 mg/ml) is pipeted onto each membrane with a wide-bore pipet tip and allowed to air-dry. Timepoints can be tested in triplicate for activity including controls. Six membranes are made for each timepoint. One set of membranes can be tested at time zero (after air-drying and prior to the accelerated stability study) using the protocol below. The other sets of membranes can be placed in a convection oven at 40° C. for 3 months. Once a week a set of six membranes can be tested using the following protocol:. each of the six membranes is placed in a 2 ml siliconized eppendorf tube. 100 μl of PBS is added to each tube. Three tubes can serve as controls and another 100 μl of PBS is added for 5 minutes. The other three tubes can have 100 μl of overnight grown bacteria (corresponding to the peptide of interest) and reacted for 5 minutes. After 5 minutes of reactions, all samples can be spun through a 0.22 μm spin filter plate (Millipore Multiscreen HTS) to remove the beads. The supernatants can then be analyzed for HRP release by placing 10 μl of supernatant+15 μl PBS+175 μl TMB substrate in a plate reader and measuring the response for 5 minutes. Samples can be compared by measuring the slope of the TMB response and averaging the triplicates of the bacteria as well as the control. The average TMB response slope can be corrected for background by subtracting the average PBS control slope. The average TMB response slope for bacteria can then be compared over the 3 month time period to the time zero sample using Student's T-test for significance.

### Example 12

### Design for a Femine Hygiene Product Comprising a Sensor

A food grade dye, such as eriogluacine (blue dye #1), can be synthesized with a reactive group(s), such a sulfonyl chloride or isothiocyanates, in order to make a diagnostic tool that can be incorporated into a product as discussed herein, such as a feminine napkin, pad, wipe, or tampon. The current non-woven materials in such products have multiple layers to provide support and absorbency. It is possible to conjugate the absorbent pad with the eriogluacine-peptide conjugate so that it would be released in the presence of, for example, BV-producing bacteria. The released dye could bind, for example, to a transparent collection window at the bottom of the feminine napkin to produce a visible color change. This design could be used for a feminine protection sensor or a diagnostic to sense conditions or states including, but not limited to vaginitis, yeast infections, pre-ovulation, genital herpes, menopause, and osteoporosis.

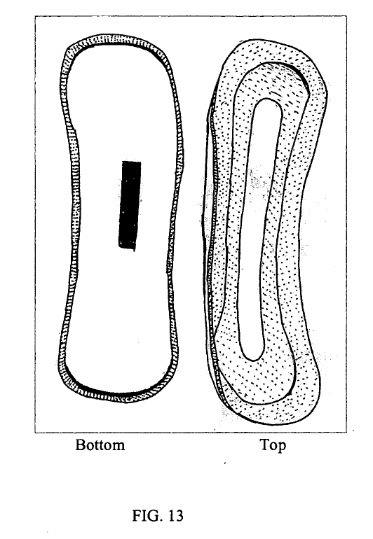

FIG. 13 illustrates one example of a design for such a feminine napkin. For example, the feminine napkin, diaper or pad may contain three layers of materials, a top liner, an absorbent material and a bottom liner that can be used to entrap enzymes and/or colorimetric components of substrates. The vaginal fluid is wicked into the non-woven material. In the presence of, for example, Gardnerella vaginalis, the dye peptide conjugate is released and allowed to bind to a transparent window at the bottom of the napkin. Dye collection can be obtained through strong ionic interactions with an ion-coated plastic or specific binding to an affinity coated plastic material. A color change indicates rge presence of specific substances in the vaginal fluid or urine. These substances may be indicators for BV, yeast infections (YEAST), genital herpes (HSV-2), pre-ovulation, menopause, or bone loss (osteoporosis).

The sensor technology described herein can be used in a feminine napkin, pad, or diaper to detect the presence of any factors, such as irritating factors, including, but not limited to, microorganisms in urinary tract infections (UTI) (e.g., E. coli, K. pneumoniae, P. mirabalis, P. aeruginosa and Enterobacter spp.), yeast infections (e.g., Candida spp.), bacterial vaginosis, trichomoniasis, host proteases and other enzymes that may cause a skin rash, diaper rash, or bed sore.

For example, many absorbent pads, napkins and diapers have at least 2-3 layers of non-woven or other absorbent and nonabsorbent material that can be used to separate a substrate from an enzyme that produces the color, for example, the napkin can have a top liner, an absorbent material and a bottom liner. An enzyme attached to a specific peptide conjugate can be coupled to polystyrene beads or agarose beads and then ink jet printed into an inner layer that contains absorbent material. Enzymes include, but are not limited to, horseradish peroxidase (HRP), phenol oxidases (e.g., laccase, CotA), galactosidase and other enzymes described herein. Alternatively, the peptide can be conjugated with a dye, such as a fluorescent or chromogenic dye, or other dyes as described herein. The bottom water-tight layer can be removed in a small area from the bottom surface and then the bottom side of the absorbent material can be printed with a substrate specific to the enzyme, such as naphthol (e.g., naphthol 5mg/ml stock in 100% methanol), TMB (tetramethyl benzidine) (e.g., TMB stabilized stock solution from US Biologics), gum guiac, or ABTS, which are common substrates for horseradish peroxidase. Alternatively the surface can be treated with ionic charges to impart a surface that can collect the simple color or fluorescent dye conjugates.

In the presence of a host or microorganism lytic enzyme, the fluid is drawn into the inner absorbent material layer. The interaction with this layer containing the peptide-enzyme-bead conjugate will release an enzyme (such as HRP) that will be free to migrate to the bottom surface which contains the substrate. Upon reacting the freely diffusible enzyme with the chromogenic substrate, a color was produced in seconds.

Sensors can be titrated up or down depending on the desired threshold by changing the concentration of the enzyme-peptide conjugate on the surface of the beads (such as 10⁶ CFU of Gardnerella vaginalis for bacterial vaginosis, 10⁵ CFU of E. coli for a urinary tract infection, 10⁶ CFU of filamentous infectious Candida albicans for a yeast infection sensor, 50-100 ng of human elastase or 1-10 ng of matrix metalloproteases (1, 2, 9 and 13) for a skin or diaper rash sensor). Alternatively, the concentration can be adjusted to the desired irritant threshold by the volume of peptide-enzyme-bead conjugate sprayed on the surface of the inner absorbent material. In some embodiments it may be ideal to place a clear sheet or window over the area that will change color in order to prevent, napkin, pad, or diaper leaks.

In one example, a diaper was cut on the bottom surface layer to provide a circular area that was then printed with TMB substrate. Beads containing HRP and a peptide that is designed to a specific irritant are injected into the inner absorbent material. In the presence of liquid that contains such an irritant, the peptides are specifically hydrolyzed and the HRP flows to the bottom surface of the pad and oxidizes the TMB substrate leading to a brilliant blue color change in minutes. Other HRP substrates like napthol can also be used.

In additional embodiments, methods using simple dyes (e.g., food grade dyes) as the calorimetric component and it can be collected on the surface, for example, with an ion exchange membrane.

### Example 13

### Incorporation of Sensors into a Lateral Flow POC Device

The liquid phase diagnostics, as described herein, can be converted to a lateral flow format as a three-in-one point of care diagnostic for lower genital tract infections. In one embodiment, the design requirements for a lateral flow point-of-care (POC) device, e.g., for doctors' offices, may include that it should: 1) work with a vaginal swab; 2) be easy to use; 3) be rapid (e.g., 5 minutes or less); and 4) be reliable (high selectivity, low false positives and negatives).

Components

The device can essentially have three membranes, a conjugate membrane, a lateral flow strip, and a wicking pad. The conjugate pad can be glass microfiber and can be printed with the HRP-Peptide-beads. The glass microfiber slows the flow of the liquid, thereby allowing time for the microbial protease to react with the HRP-peptide-beads. The lateral flow pad transfers the released HRP to the printed substrate (e.g., naphthol) and the wicking membrane at the back of the device acts as a sink to drive the liquid flow through the device.

Printing

A PC-controlled BioDot printer (AD3050 dispensing platform with 2 BioJet Quanti valves) can be used to automate the printing of both the naphthol substrate and the HRP-peptide-bead formulations. The beads can be printed with 0.1% xanthum gum as a thickening agent onto a glass microfiber conjugate pad. A line of 2% naphthol in Colloidon can be printed onto Porex K lateral flow membranes with a AIRJET QUANTI DISPENSER® spray head.

The substrates can be uniformly printed with, for example, an inkjet printer-like technology or stamped in a pattern in the presence of a thickening agent (e.g., glycerol). After placing the HRP down on the surface, the enzyme is able to flow through the porous non woven material and oxidize the substrate, thereby producing a color change that is clearly visible.

Assembly and Lamination

The conjugate pad, lateral flow, and wicking membrane can be applied to a backing membrane and then laminated using a Kinematic laminator for rapid diagnostic tests. Individual strips can be cut and placed into plastic housings that can be prototyped by a plastics manufacturer, for example, Vaupell Rapid Solutions.

### Example 14

### Incorporation of Sensors into a Consumer OTC Product

A simple and safe diagnostic sensor can be produced that can be incorporated into a three layer feminine pad as an over-the-counter (OTC) product. The color change will be sufficiently robust such that it is obvious even to the untrained observer. Blue #1 dye that is an FDA-approved food component can be used in direct contact with the vaginal fluids. The sensors, as described herein, (e.g., individual dye-peptide-bead chemistries for BV, CV, and TRIC) and the collector membranes (ICE) can first be tested individually to make certain that they are specific and sensitive enough for a consumer diagnostic. The process of validating the sensors can be repeated once the materials are incorporated into the feminine napkin or pad.

The Sensor

In one embodiment, the sensor comprises blue dye #1 conjugated to three peptides specific for BV, CV, and TRIC. The dye-peptide conjugates can be tethered to small latex beads (1 μm). The sensor can be injected into the middle layer of the feminine pad. 0.5 g of blue dye #1 (eriogluacine) can be synthesized and functionalized with maleimide.

The chemical structure of this food grade dye, N-ethyl-N-[4-[[4-[ethyl[(3-sulfophenyl)methyl]amino]phenyl](2-sulfophenyl)methylene]-2,5-cyclohexadien-1-ylidene]-3-sulfo-, inner salt, disodium salt (CAS number [3844-45-9]), is provided herein. The maleimide version of blue dye #1 can be conjugated to peptides using a dye-to-peptide molar ratio of 5:1 in phosphate buffer pH 7.5, for 1 hour at room temperature. The labeled peptide can be removed from the unincorporated dye using a PD1O column and then the dye peptide conjugate can be conjugated to polystyrene beads using EDC chemistry. The amount of beads that need to be injected into a feminine pad can be determined to give sufficient color collected at the bottom surface of the pad. The dye-peptide and/or peptide-beads ratio can be varied to optimize the proteolysis off the beads. The amount of vaginal fluids in a pad can vary greatly (100 μl-2 ml of fluid) and, therefore, the sensor should work under this range of conditions.

The Pad

Feminine pads have three layers, or discrete types, of materials. There is a slow wicking top layer, an inner layer of amorphous non-woven material, and a bottom that is leak resistant barrier. When a pad is cut in half, one finds that the inner non-woven material forms a pocket (2 layers). This material acts very much like a lateral flow device because the vaginal fluids flow in one direction away from the surface in contact with the body to the bottom non-absorbent layer. The peptides can be conjugated to the beads via reaction of the peptide N-terminal amino group with beads that have carboxyl groups on their surface using the 1-ethyl-3-(3-dimethylaminopropyl) carbodiimide hydrochloride (EDC) coupling reagent (Pierce) forming a stable amide bond in pH 4.5-7.5 buffers (100 mM MES [2-(N-morpholino) ethane sulfonic acid] or phosphate buffers). The amount of beads that need to be injected into a feminine pad can be determined to give sufficient color collected at the bottom surface of the pad. The dye-peptide and/or peptide-beads ratio can be varied to optimize the proteolysis off the beads. The amount of vaginal fluids in a pad can vary greatly (100 μl-2 ml of fluid) and, therefore, the sensor should work under this range of conditions. A range of peptide-dye conjugate and bead concentrations can be used to determine optimal signal. Any excess peptide-dye conjugate can then be washed off the beads and the beads can be rinsed until the wash buffer has an acceptable background.

Sensor/Pad Combination Product: Printing the Drops for the Beads Into a Pad

A BioDot Printer can be used to inject the simple dye-peptide-beads into the non-woven material layer of the pad. The parameters can be slightly different from the beads printed in the POC device; the syringe speed can be 100 μl/sec for the start speed, 400 μl/second for the top speed, and 10000 μl/second² for the acceleration. The amount of beads dispensed in a single droplet can be 25 μl; there can be x-axis iterations since the membrane strips can be cut individually and the drops need to be a sufficient distance apart. These iterations can be 20 mm after every pass.

The AIRJET QUANTI DISPENSER® purchased from BioDot can be used for printing both the TRISACRYL® bead conjugates and the colloidian solution with naphthol. The TRISACRYL® beads (simple dye peptide beads, or HRP peptide beads) can be printed down using a drop program while the naphthol/amyl acetate can be printed down using a line program. A minimum of 3 ml can be used for each substance in order for there to be sufficient solution within the tubing of the machine. In printing the lines with the naphthol/amyl acetate solution, the syringe speed for the dispense parameters will be 10 μl/second for both the start and top speed and 250 μl/second² for the acceleration. Within the line dispense function there can be no XY motion delay. The line coordinates under the same function can be 25.0 mm for the x coordinate and 0.0 for the y relative. The dispense rate will be 2.5 μl/cm, the line length will be 25.0 mm, and the drop pitch will be 0.304 mm. There will not be any x-axis iterations so that a single solid line can be produced.

After cutting a hole in the bottom layer of the napkin, an ICE membrane (Pall) can be inserted and a clear dressing or transparent plastic sheet can be placed over the hole to provide a leak tight window to view the color change. ICE membranes (Pall, Ann Arbor, Mich.) can be used to collect the released dye from the middle layer to the bottom surface of the pad. ICE membranes efficiently collect free dye and give a robust color signal. A blue dye (Remazol brilliant blue) can be collected efficiently from a membrane that mimics the release of the blue dye from the middle layer of a feminine pad. Other collection membranes such as P4 may be less able to collect the free dye. This prototype can be tested with artificial vaginal fluids spiked with extracts from pathogens of TRIC, BV, and CV. It is expected that the color change will be visible at the bottom surface of the pad within an hour of incubation at room temperature.

### Example 15

### Infection Sensors: Detection of Herpes Simplex Virus

An infection sensor diagnostic for the HSV virus based on the proteolytic processing required for the assembly of the mature virions was developed. In order to amplify the signal, a novel zymogen approach has been developed that comprises a specific peptide being attached to agarose or glass beads on one end with an amplification reporter enzyme such as CotA or horseradish peroxidase (HRP) on the other end.

The HSV protein UL26 is an auto-catalytically-processed 635-amino acid protease. In addition to undergoing cleavage of itself, it also hydrolyzes a gene product of an open reading frame (ORF) that is positioned directly downstream (called ICP35) Dilanni, et al., J. Biol. Chem. 268:25449-25454 (1993)). UL26 has a strong specificity for the sequence ASNAEAGALVNASSAAHVDV (SEQ ID NO: 22). The peptide is clipped between the 12^{th} amino acid (alanine) and the 13^{th} amino acid (serine). Although this specific proteolytic event is likely to be the early warning signal for genital blisters (Roizman et al., U.S. Publication No. 20020015944), it is predicted that the concentration of this protease is too low to detect without further amplification of the signal with a zymogen. This zymogen approach allows for signal amplification and early detection of the viral lytic phase which leads to blisters and genital sores.

More specifically, hydrolysis of the peptide with CotA or HRP peptide conjugates provided a leaving group to interact with substrate (ABTS, napthol) bound to a membrane. The free enzyme migrated by lateral flow, providing the formation of a line at the site where it oxidized the substrate. This zymogen approach provides a 20-2000 fold amplification of the signal, thereby, making it possible to detect the lytic cycle of the virus that has not been reliably detected by previous antibody-based methodologies.

Equivalents

While this invention has been particularly shown and described with references to preferred embodiments thereof, it will be understood by those skilled in the art that various changes in form and details may be made therein without departing from the scope of the invention encompassed by the appended claims.

Each reference cited herein is incorporated by reference in its entirety.

1. A method of assessing a condition in a female mammal, comprising:
a) exposing an unmodified substrate to a sample under conditions that will result in a modification of the substrate, wherein the unmodified substrate includes a peptide and a first calorimetric component; and
b) detecting the modification of the substrate or an absence of the modification of the substrate, wherein the modification comprises cleaving the first calorimetric component or enzyme from the substrate and results in a visible signal, wherein the modification or absence of the modification indicates a condition in the female mammal.

2. The method of claim 1, wherein the substrate is specific for a protein produced by Bacteriodes spp., Mobilincus spp., Peptostreptococcus spp., Mycoplasma hominis, Prevotella bivia and Porphyromonas spp.

3. The method of claim 1, wherein the substrate is specific for a protein produced by Trichomonas spp.

4. The method of claim 1, wherein the substrate is specific for a protein produced by Candida spp.

5-6. (canceled)

7. The method of claim 1, wherein the condition is at least one condition selected from the group consisting of candidiasis, trichomoniasis, bacterial vaginosis, a urinary tract infection, genital herpes (HSV-2), pre-ovulation, menopause, and osteoporosis.

8. The method of claim 1, further comprising measuring the pH of the sample.

9. (canceled)

10. The method of claim 1, wherein the peptide is coupled to a solid support.

11. A method of claim 10, wherein the modification of the substrate results in an increase in the visibility of the hue of the solid support.

12. A method of claim 10, wherein the peptide is covalently attached to the solid support.

13. A method of claim 10, wherein the solid support is selected from the group consisting of a bead, a sterilized material, an article that contains the sample, an article that collects the sample, a polymer, a membrane, a sponge, a disk, a scope, a filter, a foam, a cloth, a paper, a suture, and a bag.

14. The method of claim 10, wherein the solid support is selected from the group consisting of a feminine napkin, a pad, a diaper, a wipe, a swab, and a tampon.

15. The method of claim 1, wherein the first colorimetric component is a fluorescent dye, a luminescent dye or a chromogenic dye.

16. The method of claim 1, wherein the first calorimetric component is horseradish peroxidase, a phenol oxidase, luciferase, galactosidase, laccase or alkaline phosphatase.

17. The method of claim 1, wherein the unmodified substrate further includes a second colorimetric component that is dissimilar to the first colorimetric component.

18-19. (canceled)

20. The method of claim 1, wherein the substrate includes at least one member of the group consisting of: the peptide sequence PFINETYAKFC (SEQ ID NO: 1), the peptide sequence ITTTSSKHEHC (SEQ ID NO: 2), the peptide sequence KPKAFXXX (SEQ ID NO: 3), the peptide sequence VPGDPEAAEARRGQC (SEQ ID NO: 4),, the peptide sequence KPKAFLKGRR (SEQ ID NO: 5), the peptide sequence KPKAFLKVGN (SEQ ID NO: 6), the peptide sequence LYPILKKNQK (SEQ ID NO: 7), the peptide sequence KPSIKPTPPY (SEQ ID NO: 8), the peptide sequence QKTTIKKLKH (SEQ ID NO: 9), the peptide sequence TPIQIHTILH (SEQ ID NO: 10), the peptide sequence INLSKKQIYP (SEQ ID NO: 11), the peptide sequence LYPSQNPVIK (SEQ ID NO: 12), the peptide sequence NITKKSTKII (SEQ ID NO: 13), the peptide sequence NNPLPKIQKN (SEQ ID NO: 14), the peptide sequence KNPKLQDHYI (SEQ ID NO: 15), the peptide sequence QINKALKQPK (SEQ ID NO: 16), the peptide sequence QIPKSLHPIT (SEQ ID NO: 17), the peptide sequence LHNYVLLRNIL (SEQ ID NO: 18), the peptide sequence SKQQDIIKKY (SEQ ID NO: 19), and the peptide sequence NKTNKTKHAY (SEQ ID NO: 20), the peptide sequence QRTTIRRLRH (SEQ ID NO: 21), and the peptide sequence ASNAEAGALVNASSAAHVDV (SEQ ID NO: 22).

21. (canceled)

22. The method of claim 1, wherein the visible signal includes a change in hue.

23. The method of claim 1, wherein the visible signal is a loss of color.

24. The method of claim 1, wherein the sample includes a portion of vaginal fluid or urine.

25-27. (canceled)

28. A peptide comprising at least one amino acid sequence selected from the group consisting of the peptide sequence PFINETYAKFC (SEQ ID NO: 1), the peptide sequence ITTTSSKHEHC (SEQ ID NO: 2), the peptide sequence KPKAFXXX (SEQ ID NO: 3), the peptide sequence VPGDPEAAEARRGQC (SEQ ID NO: 4),, the peptide sequence KPKAFLKGRR (SEQ ID NO: 5), the peptide sequence KPKAFLKVGN (SEQ ID NO: 6), the peptide sequence LYPILKKNQK (SEQ ID NO: 7), the peptide sequence KPSIKPTPPY (SEQ ID NO: 8), the peptide sequence QKTTIKKLKH (SEQ ID NO: 9), the peptide sequence TPIQIHTILH (SEQ ID NO: 10), the peptide sequence INLSKKQIYP (SEQ ID NO: 11), the peptide sequence LYPSQNPVIK (SEQ ID NO: 12), the peptide sequence NITKKSTKII (SEQ ID NO: 13), the peptide sequence NNPLPKIQKN (SEQ ID NO: 14), the peptide sequence KNPKLQDHYI (SEQ ID NO: 15), the peptide sequence QINKALKQPK (SEQ ID NO: 16), the peptide sequence QIPKSLHPIT (SEQ ID NO: 17), the peptide sequence LHNYVLLRNIL (SEQ ID NO: 18), the peptide sequence SKQQDIIKKY (SEQ ID NO: 19), and the peptide sequence NKTNKTKHAY (SEQ ID NO: 20), the peptide sequence QRTTIRRLRH (SEQ ID NO: 21), and the peptide sequence ASNAEAGALVNASSAAHVDV (SEQ ID NO: 22).

29. A sensor for detecting the presence or absence of a protein, comprising a peptide that specifically reacts with a protein produced by a microorganism and a first colorimetric component coupled to the peptide, wherein the peptide includes at least one member selected from the group consisting of the peptide sequence PFINETYAKFC (SEQ ID NO: 1), the peptide sequence ITTTSSKHEHC (SEQ ID NO: 2), the peptide sequence KPKAFXXX (SEQ ID NO: 3), the peptide sequence VPGDPEAAEARRGQC (SEQ ID NO: 4), the peptide sequence KPKAFLKGRR (SEQ ID NO: 5), the peptide sequence KPKAFLKVGN (SEQ ID NO: 6), the peptide sequence LYPILKKNQK (SEQ ID NO: 7), the peptide sequence KPSIKPTPPY (SEQ ID NO: 8). the peptide sequence QKTTIKKLKH (SEQ ID NO: 9), the peptide sequence TPIQIHTILH (SEQ ID NO: 10), the peptide sequence INLSKKQIYP (SEQ ID NO: 11), the peptide sequence LYPSQNPVIK (SEQ ID NO: 12), the peptide sequence NITKKSTKII (SEQ ID NO: 13), the peptide sequence NNPLPKIQKN (SEQ ID NO: 14), the peptide sequence KNPKLQDHYI (SEQ ID NO: 15), the peptide sequence QINKALKQPK (SEQ ID NO: 16), the peptide sequence QIPKSLHPIT (SEQ ID NO: 17), the peptide sequence LHNYVLLRNIL (SEQ ID NO: 18). the peptide sequence SKQQDIIKKY (SEQ ID NO: 19), and the peptide sequence NKTNKTKHAY (SEQ ID NO: 20). the peptide sequence QRTTIRRLRH (SEQ ID NO: 21), and the peptide sequence ASNAEAGALVNASSAAHVDV (SEQ ID NO: 22).

30-35. (canceled)

36. The sensor of claim 29, further including a solid support for a point-of-care device, wherein the peptide is coupled to the solid support, and wherein the solid support is selected from the group consisting of a bead, a sterilized material, an article that contains the sample, an article that collects the sample, a polymer, a membrane, a sponge, a disk, a scope, a filter, a foam, a cloth, a paper, a suture, a speculum and a bag.

37. The sensor of claim 29, further including a solid support for a feminine hygiene product, wherein the peptide is coupled to the solid support, and wherein the solid support is selected from the group consisting of a feminine napkin, a pad, a diaper, a wipe, a swab, and a tampon.

38-51. (canceled)

52. The sensor of claim 37, wherein the feminine hygiene product comprises a substrate and, wherein the substrate comprises a peptide specific for each of Gardnerella vaginalis, Trichomonas vaginalis and Candida albicans.

53. A method of detecting the presence or absence of an irritating factor in a mammal, wherein said irritating factor is selected from the group consisting of bacteria, yeast, parasites, protozoa, host proteases, and enzymes, and wherein said irritating factor is detected in an absorbent pad selected from the group consisting of a feminine napkin, pad and diaper, comprising:
a) exposing an unmodified substrate to a sample under conditions that will result in a modification of the substrate, wherein the unmodified substrate includes a peptide and a first calorimetric component, wherein the first calorimetric component or is coupled to the peptide, and wherein the sample includes mammalian vaginal fluid or urine; and
b) detecting the modification of the substrate or an absence of the modification of the substrate, wherein the modification comprises cleaving the first calorimetric component or enzyme from the substrate and results in a visible signal, wherein the modification or absence of the modification indicates presence or absence of an irritating factor in the mammal.

54. A method of detecting the presence or absence of a an condition in a mammal, wherein said condition is selected from the group consisting of a urinary tract infection, yeast infection, bacterial vaginosis, candidiasis, trichomoniasis, skin rash, diaper rash and bed sore, comprising:
a) exposing an unmodified substrate to a sample under conditions that will result in a modification of the substrate, wherein the unmodified substrate includes a peptide and a first calorimetric component or an enzyme, wherein the first calorimetric component or enzyme is coupled to the peptide, and wherein the sample includes mammalian vaginal fluid or urine; and
b) detecting the modification of the substrate or an absence of the modification of the substrate, wherein the modification comprises cleaving the first calorimetric component or enzyme from the substrate and results in a visible signal, wherein the modification or absence of the modification indicates presence or absence of an irritating factor in the mammal.

55-60. (canceled)

61. The first sensor of claim 29, wherein said first colorimetric component is a food grade dye.

62. A lateral flow device for assessing a condition in a female mammal, wherein said lateral flow device comprises a lateral flow strip, a conjugate membrane, a substrate line and a wicking pad.

63. The method of claim 1, wherein the first calorimetric component is a food grade dye.

64-66. (canceled)


SEQUENCE LISTING (28 sequences)

SEQ ID NO: 1 - protein, 11 aa, Artificial Sequence
  Peptide Substrate of Gardnerella vaginalis
PFINETYAKFC

SEQ ID NO: 2 - protein, 11 aa, Artificial Sequence
  Peptide Substrate of Lactobacillus acidophilus
ITTTSSKHEHC

SEQ ID NO: 3 - protein, 8 aa, Artificial Sequence
  Peptide Substrate of Candida spp.
  VARIANT 6, 7, 8: Xaa = Any Amino Acid
KPKAFXXX

SEQ ID NO: 4 - protein, 15 aa, Artificial Sequence
  Peptide Substrate of ADAM-TS1
VPGDPEAAEARRGQC

SEQ ID NO: 5 - protein, 10 aa, Artificial Sequence
  Peptide Substrate of Candida spp.
KPKAFLKGRR

SEQ ID NO: 6 - protein, 10 aa, Artificial Sequence
  Peptide Substrate of Candida spp.
KPKAFLKVGN

SEQ ID NO: 7 - protein, 10 aa, Artificial Sequence
  Peptide Substrate of Gardnerella vaginalis
LYPILKKNQK

SEQ ID NO: 8 - protein, 10 aa, Artificial Sequence
  Peptide Substrate of Candida spp.
KPSIKPTPPY

SEQ ID NO: 9 - protein, 10 aa, Artificial Sequence
  Peptide Substrate of Candida spp.
QKTTIKKLKH

SEQ ID NO: 10 - protein, 10 aa, Artificial Sequence
  Peptide Substrate of Candida spp.
TPIQIHTILH

SEQ ID NO: 11 - protein, 10 aa, Artificial Sequence
  Peptide Substrate of Candida spp.
INLSKKQIYP

SEQ ID NO: 12 - protein, 10 aa, Artificial Sequence
  Peptide Substrate of Candida spp.
LYPSQNPVIK

SEQ ID NO: 13 - protein, 10 aa, Artificial Sequence
  Peptide Substrate of Candida spp.
NITKKSTKII

SEQ ID NO: 14 - protein, 10 aa, Artificial Sequence
  Peptide Substrate of Trichomonas vaginalis
NNPLPKIQKN

SEQ ID NO: 15 - protein, 10 aa, Artificial Sequence
  Peptide Substrate of Trichomonas vaginalis
KNPKLQDHYI

SEQ ID NO: 16 - protein, 10 aa, Artificial Sequence
  Peptide Substrate of Trichomonas vaginalis
QINKALKQPK

SEQ ID NO: 17 - protein, 10 aa, Artificial Sequence
  Peptide Substrate of Trichomonas vaginalis
QIPKSLHPIT

SEQ ID NO: 18 - protein, 11 aa, Artificial Sequence
  Peptide Substrate of Trichomonas vaginalis
LHNYVLLRNIL

SEQ ID NO: 19 - protein, 10 aa, Artificial Sequence
  Peptide Substrate of Trichomonas vaginalis
SKQQDIIKKY

SEQ ID NO: 20 - protein, 10 aa, Artificial Sequence
  Peptide Substrate of Trichomonas vaginalis
NKTNKTKHAY

SEQ ID NO: 21 - protein, 10 aa, Artificial Sequence
  Peptide Substrate of Candida spp.
QRTTIRRLRH

SEQ ID NO: 22 - protein, 20 aa, Artificial Sequence
  Peptide Substrate of Herpes simplex virus
ASNAEAGALVNASSAAHVDV

SEQ ID NO: 23 - protein, 10 aa, Artificial Sequence
  Peptide Substrate of Candida albicans
  VARIANT 6, 7, 8, 9, 10: Xaa = Any Amino Acid
KPKAFXXXXX

SEQ ID NO: 24 - protein, 10 aa, Artificial Sequence
  Peptide Substrate of Candida albicans
  VARIANT 8, 9, 10: Xaa = Any Amino Acid
KPKAFLKXXX

SEQ ID NO: 25 - DNA, 98 bp, Artificial Sequence
  nucleotide sequence
  misc_feature 3,14,28,29,30,39,40,41,45,46,47,53,54,56,63,64,69,70,71,72,73,77,78,79,87,88,89,90,95,96,97,98: n = A,T,C or G
cgngcgtgcctaantacatgcaagtcgnnnagcgagatnnngaannnctvacnnantcagttnncgbtnnnnngatnnnacgttggnnnntgaannnn

SEQ ID NO: 26 - DNA, 98 bp, Artificial Sequence
  Primer for C. albicans
catgcatgtctaagtataagcaatttatacagtgaaactgcgaatggctcattaaatcagttatcgtttatttgatagtaccttactacttggataac

SEQ ID NO: 27 - DNA, 57 bp, Artificial Sequence
  Primer for G. vaginalis
  misc_feature 20: n = A,T,C or G
cggcgtgcttaacacatgcnagtcgaacgggatctgaccagcttgctggttggtgag

SEQ ID NO: 28 - DNA, 74 bp, Artificial Sequence
  Primer for L. acidophilus
  misc_feature 68: n = A,T,C or G
cggcgtgcctaatacatgcaagtcgagcgagctgaaccaacagattcacttcggtgatgacgttgggnaacgct